(19) United States
(12) Patent Application Publication
(10) Pub. No.: US 2025/0213675 A1
(43) Pub. Date: Jul. 3, 2025

(54) IMMUNOGENS AND VACCINE COMPOSITIONS AGAINST HIV

(71) Applicant: THE HENRY M. JACKSON FOUNDATION FOR THE ADVANCEMENT OF MILITARY MEDICINE, INC., Bethesda, MD (US)

(72) Inventors: Morgane Marie ROLLAND, Silver Spring, MD (US); Eric LEWITUS, Silver Spring, MD (US); Hongjun BAI, Silver Spring, MD (US)

(21) Appl. No.: 18/852,534

(22) PCT Filed: Mar. 31, 2023
(86) PCT No.: PCT/US2023/065187

§ 371 Date: Sep. 30, 2024

Related U.S. Application Data
(60) Provisional application No. 63/437,505, filed on Jan. 6, 2023.
(60) Provisional application No. 63/325,728, filed on Mar. 31, 2022.

(51) Int. Cl. A61K 39/21; A61K 39/00; A61P 31/18; C07K 14/005; C12N 7/00; G01N 33/569; G01N 33/68

(52) U.S. Cl. CPC A61K 39/21; A61P 31/18; C07K 14/005; C12N 7/00; G01N 33/56988; G01N 33/6854; A61K 2039/53; C12N 2740/16122; C12N 2740/16134

(57) ABSTRACT

Disclosed herein are modified HIV-1 Env polypeptides comprising at least one modified hypervariable loop and isolated polynucleotides comprising a nucleotide sequence that encodes the modified HIV-1 Env polypeptides. Also disclosed herein are vaccine or immunogenic compositions for inducing an immune response in a subject against HIV, as well as a method of inducing an immune response against HIV in a subject. Further disclosed herein are methods of identifying an antibody against HIV in a sample.

### CROSS-REFERENCE TO RELATED APPLICATIONS

This application claims priority to U.S. Provisional Application No. 63/325,728, filed 31 Mar. 2022, and U.S. Provisional Application No. 63/437,505, filed 6 Jan. 2023, the contents of which are hereby incorporated by reference in its entirety.

### GOVERNMENT INTEREST

This invention was made with government support under W81XWH-18-2-0040 awarded by United States Army Medical Research and Materiel Command. The government has certain rights in the invention.

### SEQUENCE LISTING

This application contains a Sequence Listing which has been submitted electronically in XML format and is hereby incorporated by reference in its entirety. Said XML copy, created on 30 Mar. 2023, is named HMJ-180-PCT.xml and is 152,050 bytes in size.

### FIELD

This application relates generally to novel human immunodeficiency virus (HIV) immunogens and to methods and compositions related thereto. More particularly, the disclosure relates to compositions and methods for the preparation, production, and administration of isolated novel HIV immunogen nucleic acid and protein sequences suitable, for example, in certain embodiments, as vaccines against HIV and for capturing antibodies against HIV.

### BACKGROUND

An estimated 1.5 million new cases of human immunodeficiency virus (HIV) infection were diagnosed worldwide in 2021, and approximately 38.4 million people are currently living with AIDS/HIV. Although AIDS-related deaths have been dramatically reduced in recent years, an estimated 650,000 people nonetheless died from AIDS-related complications worldwide in 2021, and there remains no cure.

HIV is a retrovirus that infects CD4+ cells of the immune system, destroying or impairing their function. As the infection progresses, the immune system becomes weaker, leaving the infected person more susceptible to opportunistic infections and tumors, such as Kaposi's sarcoma, cervical cancer, lymphoma, and neurological disorders. The most advanced stage of HIV infection is acquired immunodeficiency syndrome (AIDS). It can take 10-15 years for an HIV-infected person to develop AIDS, and certain antiretroviral drugs can delay the process even further.

With respect to HIV's ability to enter into a cell, the envelope glycoprotein trimer (Env) is known to play a role in HIV virus attachment and subsequent entry into host cells. The mature Env trimer is comprised of non-covalently associated gp120-gp41 heterodimers that are formed by furin cleavage of a gp160 precursor. Kowalski M, et al. Functional regions of the envelope glycoprotein of human immunodeficiency virus type 1. Science 237, 1351-1355 (1987).

The Env trimer is the only viral protein present on the surface of virions and HIV-1 infected cells; therefore, it represents a major antibody-targeted HIV-1 antigen. Env presentation to a host immune system elicits antibody responses against many diverse Env sites. These antibodies can impact HIV-1 through various mechanisms, including direct virus neutralization and Fc-effector activities, including antibody-dependent cellular cytotoxicity (ADCC) and/or antibody-dependent cellular phagocytosis (ADCP) of infected cells.

The Env protein is metastable, heavily glycosylated, and highly diverse, due primarily to the acquisition of mutations. To counteract HIV-1 diversity, a vaccine should elicit broadly neutralizing antibodies (bnAbs) to cross-react with a variety of Env proteins. Structural biology studies have revealed the atomic details of the epitopes of numerous HIV-1 bnAbs, providing a potential roadmap for designing antigens that could integrate these bnAb epitopes. See, e.g., Wibmer, C. K., Moore, P. L. & Morris, L. HIV broadly neutralizing antibody targets. Curr. Opin. HIV AIDS 10, 135-143 (2015); and Kwong, P. D. & Mascola, J. R. HIV-1 Vaccines Based on Antibody Identification, B Cell Ontogeny, and Epitope Structure. Immunity 48, 855-871 (2018).

Although much effort has been put forth into designing effective therapeutics against HIV, currently no curative anti-retroviral drugs against HIV exist. While current antiretroviral (ART) therapies are able to control viral replication, they are unable to fully restore health or a normal immune status. ART-treated individuals still experience several co-morbidities, including increased cardiovascular disease, bone disorders and cognitive impairment. Additionally, therapy interruption leads to the re-emergence of viral replication and AIDS progression. Therefore, a need exists to develop improved therapeutics for HIV infection and AIDS.

The development of an effective vaccine against HIV remains a key challenge for fighting HIV infection. HIV-specific broadly neutralizing antibodies (bnAbs) could promote HIV remission by halting viral replication and clearing infected cells. However, there is a lack of vaccines that can induce bnAbs. Methods for effectively screening and identifying bnAbs are also needed. In addition, there is a need for reagents that match currently circulating HIV-1 sequences.

### SUMMARY

The present disclosure provides modified HIV-1 Env polypeptides and immunogenic fragments thereof comprising at least one modified hypervariable (HV) loop region. In one aspect, disclosed herein is a modified HIV-1 Env polypeptide comprising an amino acid sequence having at least 90% sequence identity, such as at least 95%, at least 98%, or at least 99% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified hypervariable (HV) loop region.

In certain embodiments, the at least one modified HV loop region is a V1 HV loop region, a V2 HV loop region, a V4 HV loop region, or a V5 HV loop region, and in certain embodiments, the modified HIV-1 Env polypeptide is of a subtype A, B, C, D, G, or M or a circulating recombinant form (CRF) 01_AE or 02_AG.

In certain embodiments, the at least one modified HV loop region is a modified V1 HV loop region chosen from SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48. In certain embodiments, the at least one modified HV loop region is a modified V2 HV loop region chosen from SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, or SEQ ID NO: 63. In certain embodiments, the at least one modified HV loop region is a modified V4 HV loop region chosen from SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, or SEQ ID NO: 79. In certain embodiments, the at least one modified HV loop region is a modified V5 HV loop region chosen from SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95.

In certain aspects of the disclosure wherein the HIV-1 is subtype A, the modified HIV-1 Env polypeptide comprises: (a) a modified V1 HV loop region chosen from SEQ ID NO: 33 or SEQ ID NO: 34; (b) a modified V2 HV loop region chosen from SEQ ID NO: 49 or SEQ ID NO: 50; (c) a modified V4 HV loop region chosen from SEQ ID NO: 64 or SEQ ID NO: 65; and (d) a modified V5 HV loop region chosen from SEQ ID NO: 80 or SEQ ID NO: 81. In certain aspects of the disclosure wherein the HIV-1 is subtype B, the modified HIV-1 Env polypeptide comprises: (a) a modified V1 HV loop region chosen from SEQ ID NO: 35 or SEQ ID NO: 36; (b) a modified V2 HV loop region chosen from SEQ ID NO: 51 or SEQ ID NO: 52; (c) a modified V4 HV loop region chosen from SEQ ID NO: 66 or SEQ ID NO: 67; and (d) a modified V5 HV loop region chosen from SEQ ID NO: 82 or SEQ ID NO: 83. In certain aspects of the disclosure wherein the HIV-1 is subtype C, the modified HIV-1 Env polypeptide comprises: (a) a modified V1 HV loop region chosen from SEQ ID NO: 37 or SEQ ID NO: 38; (b) a modified V2 HV loop region chosen from SEQ ID NO: 53 or SEQ ID NO: 54; (c) a modified V4 HV loop region chosen from SEQ ID NO: 68 or SEQ ID NO: 69; and (d) a modified V5 HV loop region chosen from SEQ ID NO: 84 or SEQ ID NO: 85. In certain aspects of the disclosure wherein the HIV-1 is subtype D, the modified HIV-1 Env polypeptide comprises: (a) a modified V1 HV loop region chosen from SEQ ID NO: 39 or SEQ ID NO: 40; (b) a modified V2 HV loop region chosen from SEQ ID NO: 55 or SEQ ID NO: 56; (c) a modified V4 HV loop region chosen from SEQ ID NO: 70 or SEQ ID NO: 71; and (d) a modified V5 HV loop region chosen from SEQ ID NO: 86 or SEQ ID NO: 87. In certain aspects of the disclosure wherein the HIV-1 is subtype G, the modified HIV-1 Env polypeptide comprises: (a) a modified V1 HV loop region chosen from SEQ ID NO: 41 or SEQ ID NO: 42; (b) a modified V2 HV loop region chosen from SEQ ID NO: 57 or SEQ ID NO: 58; (c) a modified V4 HV loop region chosen from SEQ ID NO: 72 or SEQ ID NO: 73; and (d) a modified V5 HV loop region chosen from SEQ ID NO: 88 or SEQ ID NO: 89. In certain aspects of the disclosure wherein the HIV-1 is subtype M, the modified HIV-1 Env polypeptide comprises: (a) a modified V1 HV loop region chosen from SEQ ID NO: 47 or SEQ ID NO: 48; (b) a modified V2 HV loop region of SEQ ID NO: 63; (c) a modified V4 HV loop region chosen from SEQ ID NO: 78 or SEQ ID NO: 79; and (d) a modified V5 HV loop region chosen from SEQ ID NO: 94 or SEQ ID NO: 95. In certain aspects of the disclosure wherein the HIV-1 is CRF01_AE, the modified HIV-1 Env polypeptide comprises: (a) a modified V1 HV loop region chosen from SEQ ID NO: 43 or SEQ ID NO: 44; (b) a modified V2 HV loop region chosen from SEQ ID NO: 59 or SEQ ID NO: 60; (c) a modified V4 HV loop region chosen from SEQ ID NO: 74 or SEQ ID NO: 75; and (d) a modified V5 HV loop region chosen from SEQ ID NO: 90 or SEQ ID NO: 91. In certain aspects of the disclosure wherein the HIV-1 is CRF02_AG, the modified HIV-1 Env polypeptide comprises: (a) a modified V1 HV loop region chosen from SEQ ID NO: 45 or SEQ ID NO: 46; (b) a modified V2 HV loop region chosen from SEQ ID NO: 61 or SEQ ID NO: 62; (c) a modified V4 HV loop region chosen from SEQ ID NO: 76 or SEQ ID NO: 77; and (c) a modified V5 HV loop region chosen from SEQ ID NO: 92 or SEQ ID NO: 93.

Another aspect is directed to a vaccine or immunogenic composition for inducing an immune response in a subject against HIV, wherein the vaccine or immunogenic composition comprises an optional adjuvant and at least one, such as at least two, modified HIV-1 Env polypeptide comprising an amino acid sequence having at least 90% sequence identity, such as at least 95%, at least 98%, or at least 99% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified HV loop region (or a polynucleotide (e.g., mRNA) encoding the at least one modified HIV-1 Env polypeptide).

In certain aspects, disclosed herein is an isolated polynucleotide comprising a nucleotide sequence that encodes a modified HIV-1 Env polypeptide having an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified HV loop region. In certain embodiments, the isolated polynucleotide is a mRNA or a DNA molecule. In certain embodiments, the polynucleotide is part of a vaccine or immunogenic composition comprising an optional adjuvant for inducing an immune response in a subject against HIV.

Another aspect is directed to methods of inducing an immune response against HIV in a subject, the method comprising administering to the subject the modified HIV-1 Env polypeptide or immunogenic fragment thereof, polynucleotide encoding the modified HIV-1 Env polypeptide or immunogenic fragment thereof, vaccine or immunogenic composition as disclosed herein.

In certain embodiments, disclosed herein is a method of inducing an immune response against HIV in a subject, the method comprising administering to the subject at least one polypeptide having an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118 (or an mRNA encoding the same), or an immunogenic fragment thereof, wherein the polypeptide comprises at least one modified HV loop region, and a pharmaceutically acceptable carrier. In certain embodiments, the subject is a human, and in certain embodiments, the method further comprises administering at least one adjuvant to the subject.

Another aspect is directed to methods of identifying an antibody against HIV in a sample, comprising (1) contacting the sample with at least one modified HIV-1 Env polypeptide comprising an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, and (2) determining whether a complex forms between the antibody and the at least one polypeptide or immunogenic fragment thereof, thereby identifying the antibody against HIV in the sample. In certain embodiments, the sample is a tissue sample or a body fluid sample, such as a body fluid sample selected from blood, plasma, serum, saliva, tear, urine, cerebrospinal fluid, pleural effusion, ascites, and/or peritoneal effusion. In certain embodiments, the antibody is chosen from PGT145, VRC26.25, 10-1074, PGT135, VRC01 or 3BNC117. In certain embodiments, the antibody is a broadly neutralizing antibody.

In certain embodiments, disclosed herein a modified HIV-1 Env polypeptide for use in inducing an immune response against HIV in a subject, wherein the modified HIV-1 Env polypeptide comprises an amino acid sequence having at least 90% sequence identity, such as at least 95%, at least 98%, or at least 99% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified HV loop region.

### BRIEF DESCRIPTION OF THE DRAWINGS

FIG. 1A is an illustration of epitopes of broadly neutralizing antibodies (bnAb) and nearby hypervariable (HV) loops in the HIV Env protein. The illustration provides the location of HV loops and epitopes of representative HIV-1 bnAbs, including a V2 HV loop around epitopes of V2-glycan antibodies VRC26.25 and PGT145, a V1 HV loop around epitopes of V3-glycan antibodies 10-1074 and PGT135, and a V5 HV loop around epitopes of CD4 binding site antibodies VRC01 and 3BNC117.

FIG. 1B is an illustration of epitopes of bnAbs and nearby HV loops in the HIV Env protein, illustrating the interference from long HV loops on the binding of potential broadly neutralizing antibodies targeting adjacent sites (left) and the improvement in accessibility generated after engineering shorter HV loops (right).

FIG. 2A is an illustration showing the impact of shorter loops on the antibody accessibility of epitope sites, wherein depth is an indicator of antibody accessibility of Env surface sites.

FIG. 2B is a plot showing a comparison of the depth of four representative antibodies (10-1074, PGT135, VRC01, and 3BNC117) before and after HV loop redesign in HIV B Env protein.

FIG. 2C is a plot showing a comparison of the depth of four representative antibodies (10-1074, PGT135, VRC01, and 3BNC117) before and after HV loop redesign in HIV C Env protein.

FIG. 2D is a plot showing a comparison of the depth of four representative antibodies (10-1074, PGT135, VRC01, and 3BNC117) before and after HV loop redesign in HIV CRF01_AE Env protein.

FIG. 2E is an illustration showing a structural example of a redesigned HIV CRF01_AE Env protein to improve the accessibility of 10-1074 antibody epitope sites.

FIG. 3A is a plot showing a comparison of the bnAb PGT145 sensitivity (IC₅₀ μg/ml) for HIV-1 Env in both short and long V2HV and V5HV loop regions, as described in Example 2.

FIG. 3B is a plot showing a comparison of the bnAb VRC26.25 sensitivity (IC₅₀ μg/ml) for HIV-1 Env in both short and long V2HV and V5HV loop regions, as described in Example 2.

FIG. 3C is a plot showing a comparison of the bnAb 10-1074 sensitivity (IC₅₀ μg/ml) for HIV-1 Env in both short and long V1HV and V5HV loop regions, as described in Example 2.

FIG. 3D is a plot showing a comparison of the bnAb PGT135 sensitivity (IC₅₀ μg/ml) for HIV-1 Env in both short and long V1HV and V5HV loop regions, as described in Example 2.

FIG. 3E is a plot showing a comparison of the bnAb VRC01 sensitivity (IC₅₀ μg/ml) for HIV-1 Env in both short and long V5HV and V2HV loop regions, as described in Example 2.

FIG. 3F is a plot showing a comparison of the bnAb 3BNC117 sensitivity (IC₅₀ μg/ml) for HIV-1 Env in both short and long V5HV and V2HV loop regions, as described in Example 2.

FIG. 4A is an illustration showing collision sites between the bnAb 10-1074 and both a V1HV unmodified and a V1HV redesigned loop region for an Env sequence isolated from a person living with HIV, as described in Example 4.

FIG. 4B is an illustration showing collision sites between the bnAb PGT135 and both a V1HV unmodified and a V1HV redesigned loop region for an Env sequence isolated from a person living with HIV, as described in Example 4

FIG. 4C is a plot showing a comparison of the depth (Å) of five Env variants from a person living with HIV, as described in Example 4, for the 10-1074 bnAb epitope sites, both before (left) and after (right) HV loop modification.

FIG. 4D is a plot showing a comparison of the depth (Å) of five Env variants from a person living with HIV, as described in Example 4, for the PGT135 bnAb epitope sites, both before (left) and after (right) HV loop modification.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

The present application discloses a set of novel recombinant immunogens for HIV generated by modifying the hypervariable loops of the consensus sequences of recently sampled HIV sequences in order to improve antibody-detection and/or for use as vaccine antigens. The immunogens disclosed herein surprisingly reduce or avoid hypervariable loop-mediated interference with anti-Env antibody binding by modifying the hypervariable loops and including less immunogenic sequences, while maintaining the structural integrity of HIV envelope (Env) protein.

### Definitions

In order that the present invention may be more readily understood, certain terms are first defined. Additional definitions are set forth throughout the detailed description.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps. When used herein, the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “HIV” refers to human immunodeficiency virus. HIV can be classified into two major subtypes (HIV-1 and HIV-2), each of which has many subtypes. In some embodiments, a human subject is infected with the HIV-1 or HIV-2 subtypes.

The term “antibody” refers to an immunoglobulin or antigen-binding fragment thereof, and encompasses any polypeptide comprising an antigen-binding fragment or an antigen-binding domain. The term includes but is not limited to polyclonal, monoclonal, monospecific, polyspecific, humanized, human, single-chain, chimeric, synthetic, recombinant, hybrid, mutated, grafted, and in vitro generated antibodies. Unless preceded by the word “intact,” the term “antibody” includes antibody fragments such as Fab, F(ab′)₂, Fv, scFv, Fd, dAb, and other antibody fragments that retain antigen-binding function. Unless otherwise specified, an antibody is not necessarily from any particular source, nor is it produced by any particular method.

In certain embodiments, an antibody disclosed herein is a broadly neutralizing antibody (bnAb). In certain embodiments, an antibody disclosed herein is a non-neutralizing antibody. In certain embodiments, an antibody disclosed herein is a neutralizing antibody.

In certain embodiments, an antibody can be any of the five major classes of immunoglobulins, including IgA, IgD, IgE, IgG, and IgM, or subclasses thereof, based on the identity of their heavy-chain constant domains, which are referred to as alpha, delta, epsilon, gamma, and mu, respectively. In certain embodiments, the antibody is an IgG antibody.

The basic four-chain antibody unit is a heterotetrameric glycoprotein composed of two identical light chains and two identical heavy chains. In the case of an IgG antibody, the 4-chain unit is generally about 150,000 daltons. Each light chain is linked to a heavy chain by one covalent disulfide bond, while the two heavy chain are linked to each other by one or more disulfide bonds, depending on the heavy chain isotype. Each heavy and light chain also has regularly spaced intrachain disulfide bridges.

The term “neutralizing antibody” refers to an antibody that binds to a viral antigen and directly decreases or disrupts viral entry into a cell. A neutralizing antibody may inhibit the entry of HIV with a neutralization index of, for example, >1.5 or >2.0, discussed in Kostrikis, L. G. et al., J. Virol. 1996; 70 (1): 445-458.

The term “broadly neutralizing antibody” or “bnAb” refers to an antibody that may bind to multiple versions of an antigen from different strains of a pathogen (e.g., virus), including for example multiple versions of a viral antigen (e.g., Env) from different HIV strains or isolates, each having unique genetic variations giving rise to different versions of the viral antigen (e.g., Env), and directly decrease or disrupt pathogen (e.g., HIV) entry into a cell. A bnAb may, in certain embodiments, neutralize multiple different genetic variants of a single virus (e.g., HIV).

The term “antigen” refers to a substance, such as a protein, a fragment thereof or a polysaccharide linked to a protein carrier, that when expressed in an animal or human cell or tissue is capable of triggering an immune response. The protein or fragment thereof may be glycosylated or non-glycosylated.

The term “epitope” refers to a portion of an antigen capable of being recognized and specifically bound by a particular antibody. When the antigen is a polypeptide, epitopes can be formed both from contiguous amino acids and noncontiguous amino acids juxtaposed by tertiary folding of a protein.

The term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

The term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to induce in a subject a protective immune response against an infectious disease (e.g., HIV).

The term “in some embodiments” or “in certain embodiments” or the like refers to embodiments of all aspects of the disclosure, unless the context clearly indicates otherwise.

The term “sequence identity” refers to the similarity between amino acid or nucleic acid sequences expressed in terms of the similarity between the sequences. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

The term “subject” refers to any animal, such as a mammal, including humans, non-human primates, rodents, and the like which is to be the recipient of a particular treatment.

The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. In certain embodiments, the pharmaceutically acceptable carrier or excipient is not naturally occurring.

The term “preventing” when used in the context of a disease or disease condition means prophylactic administration of a composition that stops or otherwise delays the onset of a pathological hallmark or symptom of a disease or disorder.

The term “treating” when used in the context of a disease or disease condition means ameliorating, improving or remedying a disease, disorder, or symptom of a disease or condition associated with the disease, or can mean completely or partially stopping, on a molecular level, the biochemical basis of the disease, such as halting replication of a virus, etc.

The term “vaccine composition” or “vaccine” refers to a composition that generates a protective immune response in a subject. As used herein, a “protective immune response” refers to an immune response that protects a subject from infection (prevents infection or prevents the development of disease associated with infection) or reduces the symptoms of infection (for instance an infection by HIV). Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response.

The term “vaccinate” or the like refers to the administration of a vaccine composition to generate a protective immune response in a subject, for example to a disease-causing agent such as a virus, e.g., HIV. Vaccination can occur before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccine composition.

### Modified Hypervariable Loop Regions of HIV Env Protein Epitopes

To be effective against the various highly-diverse HIV-1 strains circulating globally, an HIV-1 vaccine should be capable of eliciting bnAbs against Env. Disclosed herein are novel Env variable loop designs that mediate bnAb accessibility to Env, in particular hypervariable (HV) loops designs, i.e., the hypervariable segment that is present in the variable loops 1, 2, 4 and 5 of Env.

Known HIV-1 bnAbs cover the Env surface and are grouped in categories based on the location of their epitopes. Three epitope specificities were considered herein that are preferentially targeted by bnAbs from people living with HIV-1: (1) the Env variable loop 2 (V2)-apex epitope, (2) the glycan supersite; and (3) the CD4 binding site (CD4bs). These three epitope sites of bnAbs may be under certain functional constraints. For example, the V2-apex epitope shields V3 and stabilizes the prefusion Env trimer. The glycan-supersite epitope allows the release of V3 from the prefusion conformation to reach the co-receptors after receptor binding, and the CD4bs epitope is involved in receptor binding.

HIV-1 Env presents five variable loops and four of them (V1, V2, V4, V5) contain a hypervariable segment, called the hypervariable (HV) loop. These HV loops can be adjacent to epitope sites. The V1 HV loop (V1HV) is near the glycan-supersite epitope and is targeted by the antibodies 10-1074 and PGT135. Gristick, H. B. et al. Natively glycosylated HIV-1 Env structure reveals new mode for antibody recognition of the CD4-binding site. Nat. Struct. Mol. Biol. 23, 906-915 (2016); and Kong, L. et al. Supersite of immune vulnerability on the glycosylated face of HIV-1 envelope glycoprotein gp120. Nat. Struct. Mol. Biol. 20, 796-803 (2013).

The V2 HV loop (V2HV) is near the V2-apex epitope and is targeted by the antibodies PGT145 and VRC26.25. Lee, J. H. et al. A Broadly Neutralizing Antibody Targets the Dynamic HIV Envelope Trimer Apex via a Long, Rigidified, and Anionic β-Hairpin Structure. Immunity 46, 690-702 (2017); Liu, Q. et al. Quaternary contact in the initial interaction of CD4 with the HIV-1 envelope trimer. Nat. Struct. Mol. Biol. 24, 370-378 (2017); and Gorman, J. et al. Structure of Super-Potent Antibody CAP256-VRC26.25 in Complex with HIV-1 Envelope Reveals a Combined Mode of Trimer-Apex Recognition. Cell Rep. 31, 107488 (2020).

The V5 HV loop (V5HV) is near the CD4bs epitope and is targeted by the antibodies VRC01 and 3BNC117. Zhou, T. et al. Structural basis for broad and potent neutralization of HIV-1 by antibody VRC01. Science 329, 811-817 (2010); and Klein, F. et al. Somatic mutations of the immunoglobulin framework are generally required for broad and potent HIV-1 neutralization. Cell 153, 126-138 (2013).

These HV loops can occlude access to the adjacent bnAbs epitopes and are typically targeted by antibodies to elicit strain-specific responses rather than the more functional bnAb specificities they are shielding. Notably, these three antibody specificities accounted for more than half of the neutralizing antibodies in two natural infection cohorts. In the IAVI Protocol C, neutralization breadth was tested in 2220 samples collected from 439 people living with HIV in East Africa. Broad neutralizers were determined by a neutralization score≥1, which approximately predicted a breadth≥50% on a 105-virus panel; the three specificities corresponded to 57% of dominant specificities of top 42 neutralizers. Landais, E. et al. Broadly Neutralizing Antibody Responses in a Large Longitudinal Sub-Saharan HIV Primary Infection Cohort. PLOS Pathog. 12, e1005369 (2016). In the RV217 cohort, neutralizing responses were measured two to four years after diagnosis for 70 participants against a panel of 34 viruses and breadth was defined as recognition of 70% of strains. The three specificities account for 65% (33/51) of delineated epitope specificities. Townsley, S. M. et al. B cell engagement with HIV-1 founder virus envelope predicts development of broadly neutralizing antibodies. Cell Host Microbe 29, 564-578.e9 (2021).

This application demonstrates that modifying Env hypervariable loops provides enhanced access to bnAb epitopes on Env. As disclosed herein, Env sequences with the longest cognate hypervariable loops showed decreased sensitivity to bnAbs when compared to Env with the shortest hypervariable loops (p≤0.015). Since centralized antigens capture optimal Env features, hypervariable loops were redesigned for updated HIV-1 Env consensus sequences based on sequences sampled since 2010 from subtypes B and C and a circulating recombinant form 01_AE (CRF01_AE). V1HV, V2HV, V4HV, and V5HV were redesigned to be shorter while maintaining the integrity of the Env structure and glycan shield. Typically, the hypervariable loop was shorted by at least 3 amino acids relative to the consensus hypervariable loop sequences. In some embodiments, one or more hypervariable loops in a modified HIV-1 Env polypeptide is shortened by 1-10, 2-8, 3-15, 3-14, 3-13, 3-12, 3-11, 3-10, 3-9, 3-8, 3-7, 3-6, 3-5, or 3-4 amino acids as compared to a wild type or consensus HIV-1 Env sequence. In certain embodiments, each hypervariable loop in the modified HIV-1 Env polypeptide or immunogenic fragment or variant thereof is shortened by 1-10, 2-8, 3-15, 3-14, 3-13, 3-12, 3-11, 3-10, or 3-9 amino acids as compared to a wild type or consensus HIV-1 Env sequence.

The redesigned variable loop structures improved the antibody accessibility to the bnAbs, i.e., the closest distance between an epitope site and the antibody probe diminished, especially for the glycan-supersite epitope. Additionally, the redesigned hypervariable loops were modified to contain mainly glycine and serine that lack antibody-recognizable side chains, thus minimizing the strain-specific targeting typically associated with the Env variable loops. Accordingly, in certain embodiments, the modified HIV-1 Env polypeptide comprises one or more amino acid substitutions in one or more of the hypervariable loops. In certain embodiments, one or more amino acids in a hypervariable loop of the modified HIV-1 Env polypeptide is substituted for a glycine and/or a serine. In certain embodiments, one or more amino acids in at least 2, 3, or 4 hypervariable loops of the modified HIV-1 Env polypeptide are substituted for a glycine and/or a serine. In certain embodiments, at least 1-10, such as 2-8, amino acids in the hypervariable loops of the modified HIV-1 Env polypeptide are substituted for a glycine and/or a serine. In other embodiments, at least 2, 4, 6, 8, or 10 amino acids in a hypervariable loop of the modified HIV-1 Env polypeptide are substituted for a glycine and/or a serine. Table 1 below lists the sequences for each of the redesigned Env hypervariable loops for V1HV, V2HV, V4HV, and V5HV, including subtypes A1, B, C, D, G, CRF01_AE, CRF02_AG, and M.

TABLE 1
Redesigned Env Hypervariable Loops

| Hyper | | | |
|---|---|---|---|
| Variable | | | |
| Loop | Subtype | Original Consensus sequences | Redesigned |
| V1 | A1 | SNNNTTNTNNTNSTVTGEMRE | SNVNGSGGNATE (SEQ ID |
| | | (SEQ ID NO: 1) | NO: 33) |
| | | NVNTNNTNTNSTITGDMKE | SNVNGSGNITGDMKE (SEQ |
| | | (SEQ ID NO: 123) | ID NO: 34) |
| V1 | B | TDLNNTNTNNTNSSNSSTMEK | TDLNGSGGSMEKG (SEQ ID |
| | | G (SEQ ID NO: 2) | NO: 35) |
| | | TDLNNTTTNNTNSSSSSTMEK | TDLNGSGTMEKG (SEQ ID |
| | | G (SEQ ID NO: 124) | NO: 36) |
| V1 | C | TNVNNTNTNTNNNTNNNIKE | TNVNGSGE (SEQ ID NO: 37) |
| | | (SEQ ID NO: 3) | TNVNGSGNDTMKE (SEQ ID |
| | | TNVNVTNTNTTNNNTNNNMK | NO: 38) |
| | | E (SEQ ID NO: 125) | |
| V1 | D | TDWKNNNTTNNNTTTSEDI | TDWKGNGSGDI (SEQ ID NO: |
| | | (SEQ ID NO: 4) | 39) |
| | | TDWKNNNTTNTTTNNTTSEDI | TDWKGNGSEDI (SEQ ID NO: |
| | | (SEQ ID NO: 126) | 40) |
| V1 | G | TNVTNTNNTNNTNNNTTTVDG | TNVNGSGNGSATE (SEQ ID |
| | | KE (SEQ ID NO: 5) | NO: 41) |
| | | TNVTNNNTNNNNNNNTTVDE | TNVTGNGTVDEKE (SEQ ID |
| | | KE (SEQ ID NO: 127) | NO: 42) |
| V1 | CRF01_ | TNANLTNTNTTNNTNTTNIGNI | TNANLTGSGSGNGSIGNITD |
| | AE | TD (SEQ ID NO: 6) | (SEQ ID NO: 43) |
| | | TNANLTNTNTTNTNNTSNIGNI | TNANLTGNGSGGSIGNITD |
| | | TD (SEQ ID NO: 128) | (SEQ ID NO: 44) |
| V1 | CRF02_ | SNANNSTSNNRNITNEMKG | SNVNGSGGNMTG (SEQ ID |
| | AG | (SEQ ID NO: 7) | NO: 45) |
| | | SDVNNSSSNSNNSNISSEMKG | SDVNGSGNISSEMKG (SEQ ID |
| | | (SEQ ID NO: 129) | NO: 46) |
| V1 | M | TNANNTNTNNTNNTNTTTEEK | TNVNGSGNGSVTE (SEQ ID |
| | | E (SEQ ID NO: 8) | NO: 47) |
| | | TNVNNTNTNNTNSNNTSEEKE | TNVNGSGNGSEKE (SEQ ID |
| | | (SEQ ID NO: 130) | NO: 48) |
| V2 | A1 | NENNNSSE (SEQ ID NO: 9) | DNGSE (SEQ ID NO: 49) |
| | | NENSNNNSSE (SEQ ID NO: | NNGSE (SEQ ID NO: 50) |
| | | 131) | |
| V2 | B | DNNNNTS (SEQ ID NO: 10) | DNGS (SEQ ID NO: 51) |
| | | DNNNNNTS (SEQ ID NO: 132) | DNTS (SEQ ID NO: 52) |
| V2 | C | DNNNNSSE (SEQ ID NO: 11) | DNGSE (SEQ ID NO: 53) |
| | | NNENNNSSE (SEQ ID NO: 133) | NNGSE (SEQ ID NO: 54) |
| V2 | D | DNSNNTS (SEQ ID NO: 12) | DDNGS (SEQ ID NO: 55) |
| | | DNSNNTS (SEQ ID NO: 134) | DNTS (SEQ ID NO: 56) |
| V2 | G | GNNNNDSSS (SEQ ID NO: 13) | DDNGS (SEQ ID NO: 57) |
| | | NNNNSSS (SEQ ID NO: 135) | DNSS (SEQ ID NO: 58) |
| V2 | CRF01_ | DNNNSSE (SEQ ID NO: 14) | DNGSE (SEQ ID NO: 59) |
| | AE | DNNNNSSE (SEQ ID NO: 136) | GNSSE (SEQ ID NO: 60) |
| V2 | CRF02_ | NENNSSQ (SEQ ID NO: 15) | DNGSE (SEQ ID NO: 61) |
| | AG | DESSNSSQ (SEQ ID NO: 137) | DNSSQ (SEQ ID NO: 62) |
| V2 | M | NNNNSSE (SEQ ID NO: 16) | NNGSE (SEQ ID NO: 63) |
| | | NNNNNSSS (SEQ ID NO: 138) | |
| V4 | A1 | WNNNSTQSSNSTNSNDT (SEQ | WGNGSGNGSGNDT (SEQ ID |
| | | ID NO: 17) | NO: 64) |
| | | WNNNGTNSSNSTESNDT (SEQ | WNGNGSGNGTESNDT (SEQ |
| | | ID NO: 139) | ID NO: 65) |
| V4 | B | WNNNSTTNNNTNTNDT (SEQ | WGNGSGNGSGNDT (SEQ ID |
| | | ID NO: 18) | NO: 66) |
| | | WNNNSTNTNNNTNTNDT | WNGNGSGNGSGSNDT (SEQ |
| | | (SEQ ID NO: 140) | ID NO: 67) |
| V4 | C | YNGNGNNNNTNSNST (SEQ ID | YGNGSGNGSGNST (SEQ ID |
| | | NO: 19) | NO: 68) |
| | | YNGNGNTTNSTSNST (SEQ ID | YNGNGSGNGSGSNST (SEQ |
| | | NO: 141) | ID NO: 69) |
| V4 | D | WNNNTTTNNTTNNNET (SEQ | WGNGSGNGSGNET (SEQ ID |
| | | ID NO: 20) | NO: 70) |
| | | WNNNTTTNNTTNNNDT (SEQ | WNGNGSGNGSGSNDT (SEQ |
| | | ID NO: 142) | ID NO: 71) |
| V4 | G | LLNNNNNTTNNDT (SEQ ID | SLSGNGSGNDT (SEQ ID NO: |
| | | NO: 21) | 72) |
| | | LLNNNNNSTNDT (SEQ ID NO: | LLNGSGNGSNDT (SEQ ID |
| | | 143) | NO: 73) |
| V4 | CRF01_ | CIGNETTTEGCNGT (SEQ ID | WGNGSGNGSGNGT (SEQ ID |
| | AE | NO: 22) | NO: 74) |
| | | WNGNGNTTEGCNGT (SEQ ID | WNGNGSGNGSGSNGT (SEQ |
| | | NO: 144) | ID NO: 75) |
| V4 | CRF02_ | WNNNTTNNNNTESNDT (SEQ | WNGSGNGSGSNDT (SEQ ID |
| | AG | ID NO: 23) | NO: 76) |
| | | WNNNTTNNNNNTNSNDT | WNGNGSGNGSGSNDT (SEQ |
| | | (SEQ ID NO: 145) | ID NO: 77) |
| V4 | M | WNNNSTNNNNTNSNDT (SEQ | WGNGSGNGSGNDT (SEQ ID |
| | | ID NO: 24) | NO: 78) |
| | | WNNNTNNNNTNSNDT (SEQ | WNGNGSGNGSGSNDT (SEQ |
| | | ID NO: 146) | ID NO: 79) |
| V5 | A1 | NNNNTN (SEQ ID NO: 25) | GNGSN (SEQ ID NO: 80) |
| | | | NNGTN (SEQ ID NO: 81) |
| V5 | B | NNNTNTT (SEQ ID NO: 26) | GNGSN (SEQ ID NO: 82) |
| | | | NNGTT (SEQ ID NO: 83) |
| V5 | C | NNNNNTT (SEQ ID NO: 27) | GNGST (SEQ ID NO: 84) |
| | | NNNNNNTT (SEQ ID NO: 147) | NNGTT (SEQ ID NO: 85) |
| V5 | D | NNNSSN (SEQ ID NO: 28) | ANGSN (SEQ ID NO: 86) |
| | | NNNNSSN (SEQ ID NO: 148) | ANSSN (SEQ ID NO: 87) |
| V5 | G | NNNNTNN (SEQ ID NO: 29) | NNGSN (SEQ ID NO: 88) |
| | | NNNNNTN (SEQ ID NO: 149) | NNGTN (SEQ ID NO: 89) |
| V5 | CRF01_ | NNNNTN (SEQ ID NO: 30) | GNGSN (SEQ ID NO: 90) |
| | AE | | NNGTN (SEQ ID NO: 91) |
| V5 | CRF02_ | NNNSTN (SEQ ID NO: 31) | NNGSN (SEQ ID NO: 92) |
| | AG | | NNGTN (SEQ ID NO: 93) |
| V5 | M | NNNNTN (SEQ ID NO: 32) | GNGSN (SEQ ID NO: 94) |
| | | NNNNNTN (SEQ ID NO: 150) | NNGSN (SEQ ID NO: 95) |

For each modified loop, the anchor sites, which were relatively more conserved and located at the C/N-terminal of the hypervariable loops, were fixed. Those sites interacted with the non-hypervariable part of Env and anchored the hypervariable loops. Spacers were then iteratively tested to identify a spacer with a length that was compatible with the conformation of the rest of Env, as well as with potential N-linked glycosylation sites to keep the glycan shield of Env intact. The design iteration also sought to make C/N-anchoring sites more representative of circulating viruses.

In addition to reducing HV loop length, in certain embodiments disclosed herein, the immunogenicity of the HV loop is reduced as compared to the unmodified loop by using shorter spacers composed of less immunogenic amino acids. In certain embodiments, the redesigned spacers only contain glycine and serine except for the potential N-linked glycosylation site (PNGS). This is unlike natural HV loops, which contain various amino acids. Since glycine and serine lack antibody-recognizable side chains, the redesigned loops may in certain embodiments be less likely to act as decoys attracting strain-specific antibody responses. A recent study found that long V1V2s in CRF01_AE Env were associated with the more rapid development of autologous neutralization in participants from the RV217 cohort. Kuriakose Gift, S. et al. Evolution of Antibody Responses in HIV-1 CRF01_AE Acute Infection: Founder Envelope V1V2 Impacts the Timing and Magnitude of Autologous Neutralizing Antibodies. J. Virol. e0163522 (2023) doi:10.1128/jvi.01635-22. The results highlight the potential role of long HV loops in the development of strain-specific responses, as strain-specific responses could be considered undesirable because autologous neutralization was not associated with the development of neutralization breadth in this cohort.

In certain embodiments, the redesigned HV loops may also be combined with other strategies for improving the accessibility of bnAbs epitopes, such as fixing strain-specific glycan holes, shielding the Env trimer base, e.g., by mounting it on a liposome, or co-delivery with Gag, e.g., in an mRNA vaccine, could also help improve the accessibility of bnAbs epitopes. Shao, S. et al. Functionalization of cobalt porphyrin-phospholipid bilayers with his-tagged ligands and antigens. Nat. Chem. 7, 438-446 (2015); and Zhang, P. et al. A multiclade env-gag VLP mRNA vaccine elicits tier-2 HIV-1-neutralizing antibodies and reduces the risk of heterologous SHIV infection in macaques. Nat. Med. 27, 2234-2245 (2021). Additionally, in certain embodiments, the epitope near the redesigned HV-loops may be optimized, e.g., if mutations are found in key epitope sites, the mutated residue(s) may be reverted back to the consensus amino acid(s) at that site. Additionally, many unshielded epitopes are exposed on the post-fusion Env. Thus, a stabilized prefusion Env trimer may improve functionality, as the improved bnAbs accessibility may be insignificant in the post-fusion context.

### Modified Consensus Sequences for HIV-1 Subtypes Based on Redesigned Hypervariable Loops

Consensus sequences sampled after 2010 for each of the HIV-1 subtypes A1 (n=53), B (n=462), C (n=171), and for CRF01_AE (n=134) were generated, and consensus for sequences samples after 2000 for teach of subtypes D (n=63), G (n=62), and CFR02_AG (n=131) were generated. Each consensus was determined by the most common residue at each site, and ties were broken in the order of L, G, I, K, E, A, V, T, R, Q, S, P, N, D, W, Y, F, H, C, and then M. Sites with greater than 50% gaps in the subtype alignment were excluded. The group M consensus was a consensus of the consensus sequences for the different subtypes and CRFs; this was done without weighting by subtype/CRF prevalence. Gaps in the group M consensus, where no residue was dominant among consensus sequences for different subtypes and CRFs, were replaced with the subtype C residue.

Based on the modeled structures of each consensus using AlphaFold2 and ColabFold, the hypervariable loops were redesigned to make them short yet still compatible with structural constraints. In some embodiments, SEQ ID NOs: 96-119 as disclosed herein or immunogenic fragments or variants thereof are modified consensus sequences that reflect current HIV-1 diversity for subtypes A1, B, C, D, and G, and CRF01_AE, CRF02_AG, as well as group M.

In some embodiments, the modification of potential N-glycan sites (PNGS) retains the glycan shield, i.e., an array of N-linked glycans on the HIV-1 envelope glycoprotein, which can contribute to immunogenicity. In certain embodiments, the modified consensus sequence, such as the modified consensus sequence of an HIV-1 Env subtype B (e.g., SEQ ID NOs: 97 or 116) comprises a variable loop 3 (V3HV) of CTRPNNNTRKSIHIGPGRAFYATGDIIGDIRQAHC (SEQ ID NO: 120). In some embodiments, the hypervariable loops of the consensus sequences provided herein include a GS-linker. In some embodiments, the S in the GS-linker is replaced to form “NxS,” wherein x indicates any amino acid with the exception of proline, e.g., such that the NxS may be a PNGS site. The modified consensus sequence for each of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, and SEQ ID NO: 118 is set forth below in Table 2. Also shown in Table 2 are the original (unmodified) consensus sequences for each of SEQ ID NO: 104, SEQ ID NO: 107, SEQ ID NO: 109, SEQ ID NO: 111, SEQ ID NO: 113, SEQ ID NO: 115, SEQ ID NO: 117, and SEQ ID NO: 119, representing each of HIV-1 Env subtypes CRF01_AE, D, C, CRF02_AG, A, G, B, and M, respectively.

TABLE 3
Modified and Original Consensus Sequences with Shortened
Hypervariable Loops

| HIV-1 | Modified | |
|---|---|---|
| Sub- | or | |
| type | Original | Modified and Original Consensus Sequence |
| A1 | Modified | MRVMGTQRNCQNLLRWGTMILGMIIICSAAENLWVTVYYGVPVWK |
| | | DAETTLFCASDAKAYETEKHNVWATHACVPTDPNPQEIPLENVTE |
| | | EFNMWKNNMVEQMHTDIISLWDQSLKPCVKLTPLCVTLNCSNVNG |
| | | SGGNATEEIKNCSFNMTTELRDKKQKVYSLFYRLDIVQIDNGSEY |
| | | RLINCNTSAITQACPKVSFEPIPIHYCAPAGFAILKCNDKEFNGT |
| | | GPCKNVSTVQCTHGIKPVVSTQLLLNGSLAEGEVMIRSENITNNA |
| | | KNIIVQLNEPVKINCTRPNNNTRKSVHIGPGQAFYATGDIIGDIR |
| | | QAHCNVSRTAWNKTLQQVAKQLRKYFGNKTIIFNSSSGGDLEITT |
| | | HSFNCGGEFFYCNTSGLFNSTWGNGSGNGSGNDTITLPCRIKQII |
| | | NMWQRVGQAMYAPPIPGVIRCVSNITGLILTRDGGGNGSNETFRP |
| | | GGGDMRDNWRSELYKYKVVKIEPLGVAPTRARRRVVEREKRAVGL |
| | | GAVFLGFLGAAGSTMGAASITLTVQARQLLSGIVQQQSNLLRAIE |
| | | AQQHLLKLTVWGIKQLQARVLAVERYLKDQQLLGIWGCSGKLICT |
| | | TNVPWNSSWSNKSQDEIWDNMTWLQWDKEISNYTQIIYNLIEESQ |
| | | NQQEKNEQDLLALDKWASLWNWFDISNWLWYIKIFIMIVGGLIGL |
| | | RIVFAVLSIINRVRQGYSPLSFQTHTPNPGGLDRPGRIEEEGGEQ |
| | | GRDRSIRLVSGFLALAWDDLRSLCLFSYHRLRDFILIAARTVELL |
| | | GHSSLKGLRLGWEGLKYLWNLLLYWGRELKISAINLLDTIAIAVA |
| | | GWTDRVIEIGQRIGRAILHIPRRIRQGLERALL |
| | | (SEQ ID NO: 96) |
| B | Modified | MRVKGIRKNYQHLWRWGTMLLGMLMICSAAEQLWVTVYYGVPVWK |
| | | EATTTLFCASDAKAYDTEVHNVWATHACVPTDPNPQEVVLENVTE |
| | | NFNMWKNNMVEQMHEDIISLWDQSLKPCVKLTPLCVTLNCTDLNG |
| | | SGGSMEKGEIKNCSFNITTSIRDKVQKEYALFYKLDVVPIDNGSY |
| | | RLISCNTSVITQACPKVSFEPIPIHYCAPAGFAILKCNDKKFNGT |
| | | GPCTNVSTVQCTHGIRPVVSTQLLLNGSLAEEEVVIRSENFTDNA |
| | | KTIIVQLNESVEINCTRPNNNTRKSIHIGPGRAFYATGDIIGDIR |
| | | QAHCNISRTKWNNTLKQIVKKLREQFGNKTIIFNQSSGGDPEIVM |
| | | HSFNCGGEFFYCNTTQLFNSTWGNGSGNGSGNDTITLPCRIKQII |
| | | NMWQEVGKAMYAPPIRGQIRCSSNITGLLLTRDGGGNGSNETFRP |
| | | GGGDMRDNWRSELYKYKVVKIEPLGVAPTKAKRRVVQREKRAVGL |
| | | GAMFLGFLGAAGSTMGAASITLTVQARQLLSGIVQQQNNLLRAIE |
| | | AQQHLLQLTVWGIKQLQARVLAVERYLKDQQLLGIWGCSGKLICT |
| | | TAVPWNTSWSNKSLDEIWDNMTWMQWEREIDNYTGLIYTLIEESQ |
| | | NQQEKNEQELLELDKWASLWNWFDITNWLWYIKIFIMIVGGLIGL |
| | | RIVFAVLSIVNRVRQGYSPLSFQTRLPAPRGPDRPEGIEEEGGER |
| | | DRDRSGRLVDGFLALIWVDLRSLCLFSYHRLRDLLLIVARIVELL |
| | | GRRGWEALKYWWNLLQYWSQELKNSAVSLLNATAIAVAEGTDRII |
| | | EVVQRAGRAILHIPRRIRQGLERALL |
| | | (SEQ ID NO: 97) |
| C | Modified | MRVRGILRNYQQWWIWGILGFWMLMICNVVGNLWVTVYYGVPVWK |
| | | EAKTTLFCASDAKAYEKEVHNVWATHACVPTDPNPQEMVLENVTE |
| | | NFNMWKNDMVDQMHEDIISLWDQSLKPCVKLTPLCVTLNCTNVNG |
| | | SGEEMKNCSFNATTELRDKKKKVYALFYKLDIVPLDNGSEYRLIN |
| | | CNTSTITQACPKVSFDPIPIHYCAPAGYAILKCNNKTFNGTGPCN |
| | | NVSTVQCTHGIKPVVSTQLLLNGSLAEEEIIIRSENLTNNAKTII |
| | | VHLNESVEIVCTRPNNNTRKSIRIGPGQTFYATGDIIGDIRQAHC |
| | | NISESKWNKTLQRVSKKLAEHFPNKTIKFNPSSGGDLEITTHSFN |
| | | CRGEFFYCNTSKLFNSTYGNGSGNGSGNSTITLPCRIKQIINMWQ |
| | | EVGRAMYAPPIAGNITCKSNITGLLLTRDGGGNGSTETFRPGGGD |
| | | MRDNWRSELYKYKVVEIKPLGIAPTKAKRRVVEREKRAVGIGAVF |
| | | LGFLGAAGSTMGAASITLTVQARQLLSGIVQQQSNLLRAIEAQQH |
| | | MLQLTVWGIKQLQARVLAIERYLKDQQLLGIWGCSGKLICTTAVP |
| | | WNSSWSNKSQDDIWDNMTWMQWDREISNYTNTIYRLLEESQNQQE |
| | | KNEKDLLALDSWNNLWNWFDITNWLWYIKIFIMIVGGLIGLRIIF |
| | | AVLSIVNRVRQGYSPLSFQTLTPNPRGPDRLGRIEEEGGEQDRDR |
| | | SIRLVSGFLALAWDDLRSLCLFSYHRLRDFILVTARAVELLGRSS |
| | | LRGLQRGWEALKYLGSLVQYWGLELKKSAISLLDTIAIAVAEGTD |
| | | RIIELIQRICRAIRNIPRRIRQGFEAALQ |
| | | (SEQ ID NO: 98) |
| D | Modified | MRVRGIKRNYQHLWRWGTMLLGMLMTCSVAEQLWVTVYYGVPVWK |
| | | EATTTLFCASDAKSYKAEAHNIWATHACVPTDPNPQEIELENVTE |
| | | NFNMWKNNMVEQMHEDIISLWDQSLKPCVKLTPLCVTLNCTDWKG |
| | | NGSGDIEMKNCSFNITTEVRDKKKQVYALFYKLDVVQIDDNGSYR |
| | | LINCNTSAITQACPKVSFEPIPIHYCAPAGFAILKCNDKKFNGTG |
| | | PCKNVSTVQCTHGIKPVVSTQLLLNGSLAEEEIIIRSENLTNNAK |
| | | IIIVQLNESVTINCTRPYNNTRQSIHIGPGQAFYTTGDIIGDIRQ |
| | | AHCNISGTDWNKTLQQVAKKLGDLLNKTTIIFKPSSGGDPEITTH |
| | | SFNCGGEFFYCNTSGLFNSTWGNGSGNGSGNETITLPCRIKQIIN |
| | | MWQGVGKAMYAPPIEGLIKCSSNITGLLLTRDGGANGSNETFRPG |
| | | GGDMRDNWRSELYKYKVVRIEPLGLAPTKAKRRVVEREKRAIGLG |
| | | AMFLGFLGAAGSTMGAASLTLTVQARQLLSGIVQQQNNLLRAIEA |
| | | QQHLLQLTVWGIKQLQARVLAVERYLKDQQLLGIWGCSGKHICTT |
| | | NVPWNSSWSNKSLDEIWNNMTWMEWEREIDNYTGLIYSLIEESQI |
| | | QQEKNEQELLELDKWASLWNWFSITNWLWYIKIFIMIVGGLIGLR |
| | | IVFAVLSLVNRVRQGYSPLSFQTLLPAPRGPDRPEGIEEEGGEQG |
| | | RGRSIRLVNGFSALIWDDLRNLCLFSYHRLRDLLLIAARIVELLG |
| | | RRGWEALKYLWNLLQYWIQELKNSAISLLNTTAIAVAEGTDRVIE |
| | | IVQRAGRAILNIPRRIRQGLERALL |
| | | (SEQ ID NO: 99) |
| G | Modified | MRVKGIQRNWQHLWTWWTLILGLVIICSASNNLWVTVYYGVPVWE |
| | | DADTPLFCASDAKAYSTEKHNVWATHACVPTDPNPQEIPLENVTE |
| | | NFNMWKNNMVEQMHEDIISLWDESLKPCVKLTPLCVTLNCTNVNG |
| | | SGNGSATEELKNCSFNITTEIRDKQKKEYALFYKLDVVPIDDNGS |
| | | YRLINCNVSTIKQACPKVSFEPIPIHYCAPAGFAILKCNDKNFNG |
| | | TGPCKNVSTVQCTHGIKPVVSTQLLLNGSLAEEKIVIRSENITDN |
| | | TKVIIVQLNETIEINCTRPNNNTRKSIRIGPGQAFYATGDIIGDI |
| | | RQAHCNVSRTKWNEMLQKVKEQLKKIFNNKNITFNSSSGGDLEIT |
| | | THSFNCRGEFFYCNTSGLENNSSLSGNGSGNDTITLPCRIKQIVR |
| | | MWQRVGQAMYAPPIAGNITCRSNITGLLLTRDGGNNGSNETFRPG |
| | | GGDMRDNWRSELYKYKIVKIKPLGIAPTKARRRVVQREKRAVGLG |
| | | AVFLGFLGAAGSTMGAASITLTVQVRQLLSGIVQQQSNLLRAIEA |
| | | QQHLLQLTVWGIKQLQARVLAVERYLKDQQLLGIWGCSGKLICTT |
| | | NVPWNTSWSNKSYNEIWDNMTWIQWEREINNYTQQIYSLIEESQN |
| | | QQEKNEQDLLALDKWASLWNWFDISNWLWYIKIFIMIVGGLIGLR |
| | | IVFAVLSIVNRVRQGYSPLSFQTLTHHQREPDRPEGIEEGGGEQD |
| | | RDRSIRLVSGFLALAWDDLRSLCLFSYHRLRDFILIAARTVELLG |
| | | RSSLQGLRLGWEGLKYLWNLLLYWGQELKNSAINLLDTIAIAVAN |
| | | WTDRVIEVAQRAGRAILNIPRRIRQGLERALL |
| | | (SEQ ID NO: 100) |
| CRF0 | Modified | MRVKETQMNWPNLWKWGTLILGLVIICSASDNLWVTVYYGVPVWR |
| 1_AE | | DADTTLFCASDAKAHETEVHNVWATHACVPTDPNPQEIHLENVTE |
| | | NFNMWKNNMVEQMQEDVISLWDQSLKPCVKLTPLCVTLNCTNANL |
| | | TGSGSGNGSIGNITDEVKNCSFNMTTEIRDKKQKVYALFYKLDIV |
| | | QIDNGSEYRLINCNTSVIKQACPKISFDPIPIHYCTPAGYAILKC |
| | | NDKNFNGTGPCKNVSSVQCTHGIKPVVSTQLLLNGSLAEEEIIIR |
| | | SENLTNNAKTIIVHLNKSVEINCTRPSNNTRTSIRIGPGQVFYRT |
| | | GDIIGDIRKAYCEINGTKWNETLKQVAEKLKEHFNNKTIIFQPPS |
| | | GGDLEITMHHFNCRGEFFYCNTTKLFNSTWGNGSGNGSGNGTIIL |
| | | PCRIKQIINMWQGVGQAMYAPPISGIINCVSNITGILLTRDGGGN |
| | | GSNETFRPGGGNIKDNWRSELYKYKVVQIEPLGIAPTRAKRRVVE |
| | | REKRAVGIGAMIFGFLGAAGSTMGAASITLTVQARQLLSGIVQQQ |
| | | SNLLRAIEAQQHMLQLTVWGIKQLQARVLAVERYLKDQKFLGLWG |
| | | CSGKIICTTAVPWNSTWSNKSYEEIWNNMTWIEWEREISNYTNQI |
| | | YEILTESQNQQDKNEKDLLELDKWASLWNWFDITNWLWYIKIFIM |
| | | IVGGLIGLRIIFAVLSIVNRVRQGYSPLSFQTPTHHQREPDRPER |
| | | IEEGGGEQGRDRSVRLVSGFLALAWDDLRSLCLFSYHRLRDFILI |
| | | AARTVELLGHSSLKGLRRGWEGLKYLGNLLLYWGQELKTSAISLL |
| | | DATAIAVAGWTDRVIEVAQRAWRAILHIPRRIRQGLERALL |
| | | (SEQ ID NO: 101) |
| CRF0 | Modified | MRVMGTQRNYPLLWRWGMIIFWIIIICNAENLWVTVYYGVPVWRD |
| 2_AG | | AETTLFCASDAKAYDTEVHNVWATHACVPTDPNPQEIHLENVTEN |
| | | FNMWKNNMVEQMHEDIISLWDQSLKPCVKLTPLCVTLNCSNVNGS |
| | | GGNMTGEIKNCSFNMTTELRDKKQKVYALFYRLDVVQIDNGSEYR |
| | | LINCNTSAITQACPKVSFEPIPIHYCAPAGFAILKCNDKEFNGTG |
| | | PCKNVSTVQCTHGIKPVVSTQLLLNGSLAEEEIVIRSENITNNAK |
| | | TIIVQLNKPVKINCTRPNNNTRKSVRIGPGQTFYATGDIIGDIRQ |
| | | AHCNVSRTEWNNTLQKVATQLRKHENNKTIIFANSSGGDLEITTH |
| | | SFNCGGEFFYCNTSGLFNSTWNGSGNGSGSNDTITLQCRIKQIIN |
| | | MWQRVGQAMYAPPIPGVIRCESNITGLLLTRDGGNNGSNETFRPG |
| | | GGDMRDNWRSELYKYKVVKIEPLGVAPTHAKRRVVEREKRAVGLG |
| | | AVFLGFLGAAGSTMGAASITLTVQARQLLSGIVQQQSNLLKAIEA |
| | | QQHLLRLTVWGIKQLQARVLALERYLRDQQLLGIWGCSGKLICTT |
| | | NVPWNSSWSNKTYNDIWDNMTWLQWDKEISNYTDIIYNLIEESQN |
| | | QQEKNEQDLLALDKWASLWNWFDITNWLWYIKIFIMIVGGLIGLR |
| | | IVFAVLAIINRVRQGYSPLSFQTLTHHQREPDRPERIEEGGGEQD |
| | | RDRSVRLVSGFLALAWDDLRSLCLFSYHRLRDFVLIAARTVELLG |
| | | HSSLKGLRLGWEALKYLWNLLSYWGQELKNSAINLLDTIAIAVAN |
| | | WTDRIIEIGQRAGRAILNIPRRIRQGLERALL |
| | | (SEQ ID NO: 102) |
| M | Modified | MRVRGIQRNYQHLWRWGTLILGMLIICSAAENLWVTVYYGVPVWK |
| | | DAETTLFCASDAKAYETEVHNVWATHACVPTDPNPQEIVLENVTE |
| | | NFNMWKNNMVEQMHEDIISLWDQSLKPCVKLTPLCVTLNCTNVNG |
| | | SGNGSVTEEIKNCSFNMTTELRDKKQKVYALFYKLDIVQINNGSE |
| | | YRLINCNTSTITQACPKVSFEPIPIHYCAPAGFAILKCNDKNFNG |
| | | TGPCKNVSTVQCTHGIKPVVSTQLLLNGSLAEEEIVIRSENITNN |
| | | AKTIIVQLNESVEINCTRPNNNTRKSIRIGPGQAFYATGDIIGDI |
| | | RQAHCNISRTKWNKTLQQVAKKLRKHFNNKTIIFNSSSGGDLEIT |
| | | THSFNCGGEFFYCNTSGLFNSTWGNGSGNGSGNDTITLPCRIKQI |
| | | INMWQRVGQAMYAPPIAGNIRCESNITGLLLTRDGGGNGSNETFR |
| | | PGGGDMRDNWRSELYKYKVVKIEPLGVAPTKAKRRVVEREKRAVG |
| | | LGAVFLGFLGAAGSTMGAASITLTVQARQLLSGIVQQQSNLLRAI |
| | | EAQQHLLQLTVWGIKQLQARVLAVERYLKDQQLLGIWGCSGKLIC |
| | | TTNVPWNSSWSNKSLDEIWDNMTWMQWEREISNYTNIIYRLIEES |
| | | QNQQEKNEQDLLALDKWASLWNWFDITNWLWYIKIFIMIVGGLIG |
| | | LRIVFAVLSIVNRVRQGYSPLSFQTLTPHPRGPDRPERIEEEGGE |
| | | QGRDRSIRLVSGFLALAWDDLRSLCLFSYHRLRDFILIAARTVEL |
| | | LGRSSLKGLRRGWEGLKYLWNLLQYWGQELKNSAISLLDTIAIAV |
| | | AEWTDRVIEIGQRAGRAILNIPRRIRQGLERALL |
| | | (SEQ ID NO: 103) |
| CRF0 | Original | MRVKETQMNWPNLWKWGTLILGLVIICSASDNLWVTVYYGVPVWR |
| 1_AE | Consensus | DADTTLFCASDAKAHETEVHNVWATHACVPTDPNPQEIHLENVTE |
| | | NFNMWKNNMVEQMQEDVISLWDQSLKPCVKLTPLCVTLNCTNANL |
| | | TNTNTTNTNNTSNIGNITDEVRNCTFNMTTEIRDKKQKVYALFYK |
| | | LDIVPIDNNNNSSEYRLINCNTSVIKQACPKISFDPIPIHYCTPA |
| | | GYAILKCNDKNFNGTGPCKNVSSVQCTHGIKPVVSTQLLLNGSLA |
| | | EEEIIIRSENLTNNAKTIIVHLNKSVEINCTRPSNNTRTSIRIGP |
| | | GQVFYRTGDIIGDIRKAYCEINGTKWNETLKQVAEKLKEHFNNKT |
| | | IIFQPPSGGDLEITMHHFNCRGEFFYCNTTKLFNSTWNGNGNTTE |
| | | GCNGTIILPCRIKQIINMWQGVGQAMYAPPISGIINCVSNITGIL |
| | | LTRDGGNNNNTNETFRPGGGNIKDNWRSELYKYKVVQIEPLGIAP |
| | | TRAKRRVVEREKRAVGIGAMIFGFLGAAGSTMGAASITLTVQARQ |
| | | LLSGIVQQQSNLLRAIEAQQHLLQLTVWGIKQLQARVLAVERYLK |
| | | DQKFLGLWGCSGKIICTTAVPWNSTWSNKSYEEIWNNMTWIEWER |
| | | EISNYTNQIYEILTESQNQQDKNEKDLLELDKWASLWNWFDITNW |
| | | LWYIKIFIMIVGGLIGLRIIFAVLSIVNRVRQGYSPLSFQTPTHH |
| | | QREPDRPERIEEGGGEQGRDRSVRLVSGFLALAWDDLRSLCLFSY |
| | | HRLRDFILIAARTVELLGHSSLKGLRRGWEGLKYLGNLLLYWGQE |
| | | LKTSAISLLDATAIAVAGWTDRVIEVAQRAWRAILHIPRRIRQGL |
| | | ERALL |
| | | (SEQ ID NO: 104) |
| CRF0 | Modified | MRVKETQMNWPNLWKWGTLILGLVIICSASDNLWVTVYYGVPVWR |
| 1_AE | | DADTTLFCASDAKAHETEVHNVWATHACVPTDPNPQEIHLENVTE |
| | | NFNMWKNNMVEQMQEDVISLWDQSLKPCVKLTPLCVTLNCTNANL |
| | | TGNGSGGSIGNITDEVRNCTFNMTTEIRDKKQKVYALFYKLDIVP |
| | | IGNSSEYRLINCNTSVIKQACPKISFDPIPIHYCTPAGYAILKCN |
| | | DKNFNGTGPCKNVSSVQCTHGIKPVVSTQLLLNGSLAEEEIIIRS |
| | | ENLTNNAKTIIVHLNKSVEINCTRPSNNTRTSIRIGPGQVFYRTG |
| | | DIIGDIRKAYCEINGTKWNETLKQVAEKLKEHFNNKTIIFQPPSG |
| | | GDLEITMHHFNCRGEFFYCNTTKLFNSTWNGNGSGNGSGSNGTII |
| | | LPCRIKQIINMWQGVGQAMYAPPISGIINCVSNITGILLTRDGGN |
| | | NGTNETFRPGGGNIKDNWRSELYKYKVVQIEPLGIAPTRAKRRVV |
| | | EREKRAVGIGAMIFGFLGAAGSTMGAASITLTVQARQLLSGIVQQ |
| | | QSNLLRAIEAQQHLLQLTVWGIKQLQARVLAVERYLKDQKFLGLW |
| | | GCSGKIICTTAVPWNSTWSNKSYEEIWNNMTWIEWEREISNYTNQ |
| | | IYEILTESQNQQDKNEKDLLELDKWASLWNWFDITNWLWYIKIFI |
| | | MIVGGLIGLRIIFAVLSIVNRVRQGYSPLSFQTPTHHQREPDRPE |
| | | RIEEGGGEQGRDRSVRLVSGFLALAWDDLRSLCLFSYHRLRDFIL |
| | | IAARTVELLGHSSLKGLRRGWEGLKYLGNLLLYWGQELKTSAISL |
| | | LDATAIAVAGWTDRVIEVAQRAWRAILHIPRRIRQGLERALL |
| | | (SEQ ID NO: 105) |
| D | Modified | MRVRGIKRLYQHLWRWGTMLLGMLMICSVAENLWVTVYYGVPVWK |
| | | EATTTLFCASDAKSYKAEAHNIWATHACVPTDPNPQEIELENVTE |
| | | NFNMWKNNMVEQMHEDIISLWDQSLKPCVKLTPLCVTLNCTDWKG |
| | | NGSEDIEMKNCSFNITTEIRDKKKQVYALFYKLDVVQIDNTSYRL |
| | | INCNTSAITQACPKVTFEPIPIHYCAPAGFAILKCNDKKENGTGP |
| | | CKNVSTVQCTHGIKPVVSTQLLLNGSLAEEEIIIRSENLTNNAKI |
| | | IIVQLNESVTINCTRPYNNTRQSIHIGPGQAFYTTNDIIGDIRQA |
| | | HCNISGTEWNKTLQQVAKKLGDLLNKTTIIFKPSSGGDPEITTHS |
| | | FNCGGEFFYCNTSGLFNSTWNGNGSGNGSGSNDTITLPCRIKQII |
| | | NMWQGVGKAMYAPPIEGLIKCSSNITGLLLTRDGGANSSNETFRP |
| | | GGGDMRDNWRSELYKYKVVRIEPLGLAPTKAKRRVVEREKRAIGL |
| | | GAMFLGFLGAAGSTMGAASLTLTVQARQLLSGIVQQQNNLLRAIE |
| | | AQQHLLQLTVWGIKQLQARVLAVERYLKDQQLLGIWGCSGKHICT |
| | | TNVPWNSSWSNKSLDEIWNNMTWMEWEREIDNYTGLIYSLIEESQ |
| | | IQQEKNEQELLELDKWASLWNWFSITNWLWYIKIFIMIVGGLIGL |
| | | RIVFAVLSLVNRVRQGYSPLSFQTLLPAPRGPDRPEGIEEEGGEQ |
| | | GRGRSIRLVNGFSALIWDDLRNLCLFSYHRLRDLLLIAARIVELL |
| | | GRRGWEALKYLWNLLQYWIQELKNSAISLLNTTAIAVAEGTDRVI |
| | | EIVQRAGRAILNIPRRIRQGLERALL |
| | | (SEQ ID NO: 106) |
| D | Original | MRVRGIKRLYQHLWRWGTMLLGMLMICSVAENLWVTVYYGVPVWK |
| | Consensus | EATTTLFCASDAKSYKAEAHNIWATHACVPTDPNPQEIELENVTE |
| | | NFNMWKNNMVEQMHEDIISLWDQSLKPCVKLTPLCVTLNCTDWKN |
| | | NNTTNTTTNNTTSEDIEMKNCSFNITTEIRDKKKQVYALFYKLDV |
| | | VQIDNSNNTSYRLINCNTSAITQACPKVTFEPIPIHYCAPAGFAI |
| | | LKCNDKKFNGTGPCKNVSTVQCTHGIKPVVSTQLLLNGSLAEEEI |
| | | IIRSENLTNNAKIIIVQLNESVTINCTRPYNNTRQSIHIGPGQAF |
| | | YTTNDIIGDIRQAHCNISGTEWNKTLQQVAKKLGDLLNKTTIIFK |
| | | PSSGGDPEITTHSFNCGGEFFYCNTSGLFNSTWNNNTTTNNTTNN |
| | | NDTITLPCRIKQIINMWQGVGKAMYAPPIEGLIKCSSNITGLLLT |
| | | RDGGNNNNSSNETFRPGGGDMRDNWRSELYKYKVVRIEPLGLAPT |
| | | KAKRRVVEREKRAIGLGAMFLGFLGAAGSTMGAASLTLTVQARQL |
| | | LSGIVQQQNNLLRAIEAQQHLLQLTVWGIKQLQARVLAVERYLKD |
| | | QQLLGIWGCSGKHICTTNVPWNSSWSNKSLDEIWNNMTWMEWERE |
| | | IDNYTGLIYSLIEESQIQQEKNEQELLELDKWASLWNWFSITNWL |
| | | WYIKIFIMIVGGLIGLRIVFAVLSLVNRVRQGYSPLSFQTLLPAP |
| | | RGPDRPEGIEEEGGEQGRGRSIRLVNGFSALIWDDLRNLCLFSYH |
| | | RLRDLLLIAARIVELLGRRGWEALKYLWNLLQYWIQELKNSAISL |
| | | LNTTAIAVAEGTDRVIEIVQRAGRAILNIPRRIRQGLERALL |
| | | (SEQ ID NO: 107) |
| C | Modified | MRVRGILRNYQQWWIWGILGFWMLMICNVVGNLWVTVYYGVPVWK |
| | | EAKTTLFCASDAKAYEKEVHNVWATHACVPTDPNPQEMVLENVTE |
| | | NFNMWKNDMVDQMHEDIISLWDQSLKPCVKLTPLCVTLNCTNVNG |
| | | SGNDTMKEEMKNCSFNATTEIRDKKKKVYALFYKLDIVPLNNGSE |
| | | YRLINCNTSAITQACPKVSFDPIPIHYCAPAGYAILKCNNKTFNG |
| | | TGPCNNVSTVQCTHGIKPVVSTQLLLNGSLAEEEIIIRSENLTNN |
| | | AKTIIVHLNESVEIVCTRPNNNTRKSIRIGPGQTFYATGDIIGDI |
| | | RQAHCNISESKWNKTLQRVSKKLKEHFPNKTIKFNPSSGGDLEIT |
| | | THSFNCRGEFFYCNTSKLFNGTYNGNGSGNGSGSNSTITLPCRIK |
| | | QIINMWQEVGRAMYAPPIAGNITCKSNITGLLLTRDGGNNGTTET |
| | | FRPGGGDMRDNWRSELYKYKVVEIKPLGIAPTKAKRRVVEREKRA |
| | | VGIGAVFLGFLGAAGSTMGAASITLTVQARQLLSGIVQQQSNLLR |
| | | AIEAQQHMLQLTVWGIKQLQARVLAIERYLKDQQLLGIWGCSGKL |
| | | ICTTAVPWNSSWSNKSQEEIWDNMTWMQWDREISNYTDTIYRLLE |
| | | ESQNQQEKNEKDLLALDSWNNLWNWFDITNWLWYIKIFIMIVGGL |
| | | IGLRIIFAVLSIVNRVRQGYSPLSFQTLTPNPRGPDRLGRIEEEG |
| | | GEQDRDRSIRLVSGFLALAWDDLRSLCLFSYHRLRDFILVTARAV |
| | | ELLGRSSLRGLQRGWEALKYLGSLVQYWGLELKKSAISLLDTIAI |
| | | AVAEGTDRIIELIQRICRAIRNIPRRIRQGFEAALQ |
| | | (SEQ ID NO: 108) |
| C | Original | MRVRGILRNYQQWWIWGILGFWMLMICNVVGNLWVTVYYGVPVWK |
| | | EAKTTLFCASDAKAYEKEVHNVWATHACVPTDPNPQEMVLENVTE |
| | | NFNMWKNDMVDQMHEDIISLWDQSLKPCVKLTPLCVTLNCTNVNV |
| | | TNTNTTNNNTNNNMKEEMKNCSFNATTEIRDKKKKVYALFYKLDI |
| | | VPLNNENNNSSEYRLINCNTSAITQACPKVSFDPIPIHYCAPAGY |
| | | AILKCNNKTFNGTGPCNNVSTVQCTHGIKPVVSTQLLLNGSLAEE |
| | | EIIIRSENLTNNAKTIIVHLNESVEIVCTRPNNNTRKSIRIGPGQ |
| | | TFYATGDIIGDIRQAHCNISESKWNKTLQRVSKKLKEHFPNKTIK |
| | | FNPSSGGDLEITTHSFNCRGEFFYCNTSKLFNGTYNGNGNTTNST |
| | | SNSTITLPCRIKQIINMWQEVGRAMYAPPIAGNITCKSNITGLLL |
| | | TRDGGNNNNNNTTETFRPGGGDMRDNWRSELYKYKVVEIKPLGIA |
| | | PTKAKRRVVEREKRAVGIGAVFLGFLGAAGSTMGAASITLTVQAR |
| | | QLLSGIVQQQSNLLRAIEAQQHMLQLTVWGIKQLQARVLAIERYL |
| | | KDQQLLGIWGCSGKLICTTAVPWNSSWSNKSQEEIWDNMTWMQWD |
| | | REISNYTDTIYRLLEESQNQQEKNEKDLLALDSWNNLWNWFDITN |
| | | WLWYIKIFIMIVGGLIGLRIIFAVLSIVNRVRQGYSPLSFQTLTP |
| | | NPRGPDRLGRIEEEGGEQDRDRSIRLVSGFLALAWDDLRSLCLFS |
| | | YHRLRDFILVTARAVELLGRSSLRGLQRGWEALKYLGSLVQYWGL |
| | | ELKKSAISLLDTIAIAVAEGTDRIIELIQRICRAIRNIPRRIRQG |
| | | FEAALQ |
| | | (SEQ ID NO: 109) |
| CRF0 | Modified | MRVMRIQRNYPLLWRWGMIIFWIMIICNAENLWVTVYYGVPVWRD |
| 2_AG | | AETTLFCASDAKAYDTEVHNVWATHACVPTDPNPQEIHLENVTEN |
| | | FNMWKNNMVEQMHEDIISLWDQSLKPCVKLTPLCVTLNCSDVNGS |
| | | GNISSEMKGEIKNCSFNMTTELRDKKQKVYALFYRLDVVQIDNSS |
| | | QYRLINCNTSAITQACPKVSFEPIPIHYCAPAGFAILKCNDKEFN |
| | | GTGPCKNVSTVQCTHGIKPVVSTQLLLNGSLAEEEIVIRSENITN |
| | | NAKTIIVQLNEPVKINCTRPNNNTRKSVRIGPGQTFYATGDIIGD |
| | | IRQAHCNVSKTEWNNTLQKVATQLRKYFNNKTIIFANSSGGDLEI |
| | | TTHSFNCGGEFFYCNTSGLFNSTWNGNGSGNGSGSNDTITLQCRI |
| | | KQIINMWQRVGQAMYAPPIPGVIRCESNITGLLLTRDGGNNGTNE |
| | | TFRPGGGDMRDNWRSELYKYKVVKIEPLGVAPTHAKRRVVEREKR |
| | | AVGLGAVFLGFLGAAGSTMGAASITLTVQARQLLSGIVQQQSNLL |
| | | KAIEAQQHLLKLTVWGIKQLQARVLALERYLRDQQLLGIWGCSGK |
| | | LICTTTVPWNSSWSNKTYNDIWDNMTWLQWDKEISNYTDIIYNLI |
| | | EESQNQQEKNEQDLLALDKWASLWNWFDITNWLWYIKIFIMIVGG |
| | | LIGLRIVFAVLNIINRVRQGYSPLSFQTLTHHQREPDRPERIEEG |
| | | GGEQDRDRSVRLVSGFLALAWDDLRSLCLFSYHRLRDFVLIAART |
| | | VELLGHSSLKGLRLGWEALKYLWNLLSYWGQELKNSAINLLDTIA |
| | | IAVANWTDRVIEIGQRAGRAILNIPRRIRQGLERALL |
| | | (SEQ ID NO: 110) |
| CRF0 | Original | MRVMRIQRNYPLLWRWGMIIFWIMIICNAENLWVTVYYGVPVWRD |
| 2_AG | Consensus | AETTLFCASDAKAYDTEVHNVWATHACVPTDPNPQEIHLENVTEN |
| | | FNMWKNNMVEQMHEDIISLWDQSLKPCVKLTPLCVTLNCSDVNNS |
| | | SSNSNNSNISSEMKGEIKNCSFNMTTELRDKKQKVYALFYRLDVV |
| | | QIDESSNSSQYRLINCNTSAITQACPKVSFEPIPIHYCAPAGFAI |
| | | LKCNDKEFNGTGPCKNVSTVQCTHGIKPVVSTQLLLNGSLAEEEI |
| | | VIRSENITNNAKTIIVQLNEPVKINCTRPNNNTRKSVRIGPGQTF |
| | | YATGDIIGDIRQAHCNVSKTEWNNTLQKVATQLRKYENNKTIIFA |
| | | NSSGGDLEITTHSFNCGGEFFYCNTSGLFNNTWNNNTTNNNNNTN |
| | | SNDTITLQCRIKQIINMWQRVGQAMYAPPIPGVIRCESNITGLLL |
| | | TRDGGNNNSTNETFRPGGGDMRDNWRSELYKYKVVKIEPLGVAPT |
| | | HAKRRVVEREKRAVGLGAVFLGFLGAAGSTMGAASITLTVQARQL |
| | | LSGIVQQQSNLLKAIEAQQHLLKLTVWGIKQLQARVLALERYLRD |
| | | QQLLGIWGCSGKLICTTTVPWNSSWSNKTYNDIWDNMTWLQWDKE |
| | | ISNYTDIIYNLIEESQNQQEKNEQDLLALDKWASLWNWFDITNWL |
| | | WYIKIFIMIVGGLIGLRIVFAVLNIINRVRQGYSPLSFQTLTHHQ |
| | | REPDRPERIEEGGGEQDRDRSVRLVSGFLALAWDDLRSLCLFSYH |
| | | RLRDFVLIAARTVELLGHSSLKGLRLGWEALKYLWNLLSYWGQEL |
| | | KNSAINLLDTIAIAVANWTDRVIEIGQRAGRAILNIPRRIRQGLE |
| | | RALL |
| | | (SEQ ID NO: 111) |
| A1 | Modified | MRVMGTQRNCQNLLRWGTMILGMIIICSAAENLWVTVYYGVPVWK |
| | | DAETTLFCASDAKAYETEKHNVWATHACVPTDPNPQEIPLENVTE |
| | | EFNMWKNNMVEQMHTDIISLWDQSLKPCVKLTPLCVTLNCSNVNG |
| | | SGNITGDMKEEMKNCSFNMTTELRDKKQKVYSLFYKLDIVQINNG |
| | | SEYRLINCNTSAITQACPKVSFEPIPIHYCAPAGFAILKCNDKEF |
| | | NGTGPCKNVSTVQCTHGIKPVVSTQLLLNGSLAEGEVMIRSENIT |
| | | DNAKNIIVQLNEPVKINCTRPNNNTRKSVRIGPGQAFYATGDIIG |
| | | DIRQAHCNVSKSEWNKTLQQVAKQLRKYFGNKTIIFTNSSGGDLE |
| | | ITTHSFNCGGEFFYCNTSGLFNSTWNGNGSGNGTESNDTITLPCR |
| | | IKQIINMWQRVGQAMYAPPIPGVIRCVSNITGLILTRDGGNNGTN |
| | | ETFRPGGGDMRDNWRSELYKYKVVKIEPLGVAPTRAKRRVVEREK |
| | | RAVGLGAVFLGFLGAAGSTMGAASITLTVQARQLLSGIVQQQSNL |
| | | LRAIEAQQHLLKLTVWGIKQLQARVLAVERYLKDQQLLGIWGCSG |
| | | KLICTTNVPWNSSWSNKSYDEIWDNMTWLQWDKEISNYTQIIYRL |
| | | IEESQNQQEKNEQDLLALDKWASLWNWFDISNWLWYIKIFIMIVG |
| | | GLIGLRIVFAVLSIINRVRQGYSPLSFQTHTPNPGGLDRPGRIEE |
| | | EGGEQDRDRSIRLVSGFLALAWDDLRSLCLFSYHRLRDFILIAAR |
| | | TVELLGHSSLKGLRLGWEGLKYLWNLLLYWGRELKISAINLFDTI |
| | | AIAVAGWTDRVIEIGQRIGRAILHIPRRIRQGLERALL |
| | | (SEQ ID NO: 112) |
| A1 | Original | MRVMGTQRNCQNLLRWGTMILGMIIICSAAENLWVTVYYGVPVWK |
| | Consensus | DAETTLFCASDAKAYETEKHNVWATHACVPTDPNPQEIPLENVTE |
| | | EFNMWKNNMVEQMHTDIISLWDQSLKPCVKLTPLCVTLNCSNVNT |
| | | NNTNTNSTITGDMKEEMKNCSFNMTTELRDKKQKVYSLFYKLDIV |
| | | QINENSNNNSSEYRLINCNTSAITQACPKVSFEPIPIHYCAPAGF |
| | | AILKCNDKEFNGTGPCKNVSTVQCTHGIKPVVSTQLLLNGSLAEG |
| | | EVMIRSENITDNAKNIIVQLNEPVKINCTRPNNNTRKSVRIGPGQ |
| | | AFYATGDIIGDIRQAHCNVSKSEWNKTLQQVAKQLRKYFGNKTII |
| | | FTNSSGGDLEITTHSFNCGGEFFYCNTSGLFNSTWNNNGTNSSNS |
| | | TESNDTITLPCRIKQIINMWQRVGQAMYAPPIPGVIRCVSNITGL |
| | | ILTRDGGNNNNTNETFRPGGGDMRDNWRSELYKYKVVKIEPLGVA |
| | | PTRAKRRVVEREKRAVGLGAVFLGFLGAAGSTMGAASITLTVQAR |
| | | QLLSGIVQQQSNLLRAIEAQQHLLKLTVWGIKQLQARVLAVERYL |
| | | KDQQLLGIWGCSGKLICTTNVPWNSSWSNKSYDEIWDNMTWLQWD |
| | | KEISNYTQUIYRLIEESQNQQEKNEQDLLALDKWASLWNWFDISN |
| | | WLWYIKIFIMIVGGLIGLRIVFAVLSIINRVRQGYSPLSFQTHTP |
| | | NPGGLDRPGRIEEEGGEQDRDRSIRLVSGFLALAWDDLRSLCLFS |
| | | YHRLRDFILIAARTVELLGHSSLKGLRLGWEGLKYLWNLLLYWGR |
| | | ELKISAINLFDTIAIAVAGWTDRVIEIGQRIGRAILHIPRRIRQG |
| | | LERALL |
| | | (SEQ ID NO: 113) |
| G | Modified | MRVKGIQRNWQHLWKWGTLILGLMIICSASENLWVTVYYGVPVWE |
| | | DADTPLFCASDAKAYSTEKHNVWATHACVPTDPNPQEIPLENVTE |
| | | NFNMWKNNMVEQMHEDIISLWDESLKPCVKLTPLCVTLNCTNVTG |
| | | NGTVDEKEELKNCSFNITTEIRDKKKKEYALFYKLDVVPIDNSSY |
| | | RLINCNVSTIKQACPKVSFDPIPIHYCAPAGFAILKCRDKNFNGT |
| | | GPCKNVSTVQCTHGIKPVVSTQLLLNGSLAEEEIVIRSENITDNA |
| | | KIIIVQLNETIEINCTRPNNNTRKSIRIGPGQAFYATGDIIGDIR |
| | | QAHCNVSKTKWNEMLQKVKTKLKEYFNNKNITFASSSGGDLEITT |
| | | HSFNCRGEFFYCNTSGLENNSLLNGSGNGSNDTITLPCRIKQIIR |
| | | MWQRVGQAMYAPPIAGNITCRSNITGLLLTRDGGNNGTNETFRPG |
| | | GGDMRDNWRSELYKYKVVKIKPLGVAPTKARRRVVGREKRAVGLG |
| | | AVFLGFLGAAGSTMGAASITLTVQVRQLLSGIVQQQSNLLRAIEA |
| | | QQHLLKLTVWGIKQLQARVLAVERYLKDQQLLGIWGCSGKLICTT |
| | | TVPWNTSWSNKSYEEIWDNMTWIQWEREISNYTQQIYSLIEESQI |
| | | QQEKNEQDLLALDKWASLWNWFDISNWLWYIKIFIMIVGGLIGLR |
| | | IVFAVLNIVNRVRQGYSPLSFQTLTHHQREPDRPERIEEGGGEQD |
| | | RDRSIRLVSGFLALAWDDLRSLCLFSYHRLRDFVLIAARTVELLG |
| | | RSSLQGLRLGWEGLKYLWNLLLYWGQELKNSAINLLDTVAIAVAN |
| | | WTDRVIEVAQRAGRAILNIPRRIRQGLERALL |
| | | (SEQ ID NO: 114) |
| G | Original | MRVKGIQRNWQHLWKWGTLILGLMIICSASENLWVTVYYGVPVWE |
| | | DADTPLFCASDAKAYSTEKHNVWATHACVPTDPNPQEIPLENVTE |
| | | NFNMWKNNMVEQMHEDIISLWDESLKPCVKLTPLCVTLNCTNVTN |
| | | NNTNNNNNNNTTVDEKEELKNCSFNITTEIRDKKKKEYALFYKLD |
| | | VVPINNNNSSSYRLINCNVSTIKQACPKVSFDPIPIHYCAPAGFA |
| | | ILKCRDKNFNGTGPCKNVSTVQCTHGIKPVVSTQLLLNGSLAEEE |
| | | IVIRSENITDNAKIIIVQLNETIEINCTRPNNNTRKSIRIGPGQA |
| | | FYATGDIIGDIRQAHCNVSKTKWNEMLQKVKTKLKEYFNNKNITF |
| | | ASSSGGDLEITTHSFNCRGEFFYCNTSGLENNSLLNNNNNSTNDT |
| | | ITLPCRIKQIIRMWQRVGQAMYAPPIAGNITCRSNITGLLLTRDG |
| | | GNNNNNTNETFRPGGGDMRDNWRSELYKYKVVKIKPLGVAPTKAR |
| | | RRVVGREKRAVGLGAVFLGFLGAAGSTMGAASITLTVQVRQLLSG |
| | | IVQQQSNLLRAIEAQQHLLKLTVWGIKQLQARVLAVERYLKDQQL |
| | | LGIWGCSGKLICTTTVPWNTSWSNKSYEEIWDNMTWIQWEREISN |
| | | YTQQIYSLIEESQIQQEKNEQDLLALDKWASLWNWFDISNWLWYI |
| | | KIFIMIVGGLIGLRIVFAVLNIVNRVRQGYSPLSFQTLTHHQREP |
| | | DRPERIEEGGGEQDRDRSIRLVSGFLALAWDDLRSLCLFSYHRLR |
| | | DFVLIAARTVELLGRSSLQGLRLGWEGLKYLWNLLLYWGQELKNS |
| | | AINLLDTVAIAVANWTDRVIEVAQRAGRAILNIPRRIRQGLERAL |
| | | L |
| | | (SEQ ID NO: 115) |
| B | Modified | MRVKGIRKNYQHLWRWGTMLLGMLMICSAAEQLWVTVYYGVPVWK |
| | | EATTTLFCASDAKAYDTEVHNVWATHACVPTDPNPQEVVLENVTE |
| | | NFNMWKNNMVEQMHEDIISLWDQSLKPCVKLTPLCVTLNCTDLNG |
| | | SGTMEKGEIKNCSFNITTSIRDKVQKEYALFYKLDVVPIDNTSYR |
| | | LISCNTSVITQACPKVSFEPIPIHYCAPAGFAILKCNDKKFNGTG |
| | | PCTNVSTVQCTHGIRPVVSTQLLLNGSLAEEEVVIRSENFTDNAK |
| | | TIIVQLNESVEINCTRPNNNTRKSIHIGPGRAFYATGDIIGDIRQ |
| | | AHCNISRTKWNNTLKQIVKKLREQFGNKTIVFNQSSGGDPEIVMH |
| | | SFNCGGEFFYCNTTQLFNSTWNGNGSGNGSGSNDTITLPCRIKQI |
| | | INMWQEVGKAMYAPPIRGQIRCSSNITGLLLTRDGGNNGTTETFR |
| | | PGGGDMRDNWRSELYKYKVVKIEPLGVAPTKAKRRVVQREKRAVG |
| | | IGAMFLGFLGAAGSTMGAASITLTVQARQLLSGIVQQQNNLLRAI |
| | | EAQQHLLQLTVWGIKQLQARVLAVERYLKDQQLLGIWGCSGKLIC |
| | | TTAVPWNTSWSNKSLDEIWDNMTWMQWEREIDNYTGLIYTLIEES |
| | | QNQQEKNEQELLELDKWASLWNWFDITNWLWYIKIFIMIVGGLIG |
| | | LRIVFTVLSIVNRVRQGYSPLSFQTRLPAPRGPDRPEGIEEEGGE |
| | | RDRDRSGRLVDGFLALIWVDLRSLCLFSYHRLRDLLLIVARIVEL |
| | | LGRRGWEALKYWWNLLQYWSQELKNSAVSLLNATAIAVAEGTDRI |
| | | IEVVQRAGRAILHIPRRIRQGLERALL |
| | | (SEQ ID NO: 116) |
| B | Original | MRVKGIRKNYQHLWRWGTMLLGMLMICSAAEQLWVTVYYGVPVWK |
| | Consensus | EATTTLFCASDAKAYDTEVHNVWATHACVPTDPNPQEVVLENVTE |
| | | NFNMWKNNMVEQMHEDIISLWDQSLKPCVKLTPLCVTLNCTDLNN |
| | | TTTNNTNSSSSSTMEKGEIKNCSFNITTSIRDKVQKEYALFYKLD |
| | | VVPIDNNNNNTSYRLISCNTSVITQACPKVSFEPIPIHYCAPAGF |
| | | AILKCNDKKFNGTGPCTNVSTVQCTHGIRPVVSTQLLLNGSLAEE |
| | | EVVIRSENFTDNAKTIIVQLNESVEINCTRPNNNTRKSIHIGPGR |
| | | AFYATGDIIGDIRQAHCNISRTKWNNTLKQIVKKLREQFGNKTIV |
| | | ENQSSGGDPEIVMHSFNCGGEFFYCNTTQLFNSTWNNNSTNTNNN |
| | | TNTNDTITLPCRIKQIINMWQEVGKAMYAPPIRGQIRCSSNITGL |
| | | LLTRDGGNNNTNTTETFRPGGGDMRDNWRSELYKYKVVKIEPLGV |
| | | APTKAKRRVVQREKRAVGIGAMFLGFLGAAGSTMGAASITLTVQA |
| | | RQLLSGIVQQQNNLLRAIEAQQHLLQLTVWGIKQLQARVLAVERY |
| | | LKDQQLLGIWGCSGKLICTTAVPWNTSWSNKSLDEIWDNMTWMQW |
| | | EREIDNYTGLIYTLIEESQNQQEKNEQELLELDKWASLWNWFDIT |
| | | NWLWYIKIFIMIVGGLIGLRIVFTVLSIVNRVRQGYSPLSFQTRL |
| | | PAPRGPDRPEGIEEEGGERDRDRSGRLVDGFLALIWVDLRSLCLF |
| | | SYHRLRDLLLIVARIVELLGRRGWEALKYWWNLLQYWSQELKNSA |
| | | VSLLNATAIAVAEGTDRIIEVVQRAGRAILHIPRRIRQGLERALL |
| | | (SEQ ID NO: 117) |
| M | Modified | MRVRGIQRNYQHLWRWGTLILGMLIICSAAENLWVTVYYGVPVWK |
| | | DAETTLFCASDAKAYETEVHNVWATHACVPTDPNPQEIVLENVTE |
| | | NFNMWKNNMVEQMHEDIISLWDQSLKPCVKLTPLCVTLNCTNVNG |
| | | SGNGSEKEEIKNCSFNMTTELRDKKQKVYALFYKLDIVQINNGSE |
| | | YRLINCNTSTITQACPKVSFEPIPIHYCAPAGFAILKCNDKNFNG |
| | | TGPCKNVSTVQCTHGIKPVVSTQLLLNGSLAEEEIVIRSENITNN |
| | | AKTIIVQLNESVEINCTRPNNNTRKSIRIGPGQAFYATGDIIGDI |
| | | RQAHCNISKTKWNKTLQQVAKKLRKYFNNKTIIFNSSSGGDLEIT |
| | | THSFNCGGEFFYCNTSGLFNSTWNGNGSGNGSGSNDTITLPCRIK |
| | | QIINMWQRVGQAMYAPPIAGNIRCESNITGLLLTRDGGNNGSNET |
| | | FRPGGGDMRDNWRSELYKYKVVKIEPLGVAPTKAKRRVVEREKRA |
| | | VGLGAVFLGFLGAAGSTMGAASITLTVQARQLLSGIVQQQSNLLR |
| | | AIEAQQHLLQLTVWGIKQLQARVLAVERYLKDQQLLGIWGCSGKL |
| | | ICTTNVPWNSSWSNKSLDEIWDNMTWMQWEREISNYTNIIYRLIE |
| | | ESQNQQEKNEQDLLELDKWASLWNWFDITNWLWYIKIFIMIVGGL |
| | | IGLRIVFAVLSIVNRVRQGYSPLSFQTLTPHPRGPDRPERIEEEG |
| | | GEQGRDRSIRLVSGFLALAWDDLRSLCLFSYHRLRDFILIAARTV |
| | | ELLGRSSLKGLRRGWEGLKYLWNLLQYWGQELKNSAISLLDTTAI |
| | | AVAEWTDRVIEIVQRAGRAILNIPRRIRQGLERALL |
| | | (SEQ ID NO: 118) |
| M | Original | MRVRGIQRNYQHLWRWGTLILGMLIICSAAENLWVTVYYGVPVWK |
| | Consensus | DAETTLFCASDAKAYETEVHNVWATHACVPTDPNPQEIVLENVTE |
| | | NFNMWKNNMVEQMHEDIISLWDQSLKPCVKLTPLCVTLNCTNVNN |
| | | TNTNNTNSNNTSEEKEEIKNCSFNMTTELRDKKQKVYALFYKLDI |
| | | VQINNNNNSSSYRLINCNTSTITQACPKVSFEPIPIHYCAPAGFA |
| | | ILKCNDKNFNGTGPCKNVSTVQCTHGIKPVVSTQLLLNGSLAEEE |
| | | IVIRSENITNNAKTIIVQLNESVEINCTRPNNNTRKSIRIGPGQA |
| | | FYATGDIIGDIRQAHCNISKTKWNKTLQQVAKKLRKYFNNKTIIF |
| | | NSSSGGDLEITTHSFNCGGEFFYCNTSGLFNSTWNNNTNNNNTNS |
| | | NDTITLPCRIKQIINMWQRVGQAMYAPPIAGNIRCESNITGLLLT |
| | | RDGGNNNNNTNETFRPGGGDMRDNWRSELYKYKVVKIEPLGVAPT |
| | | KAKRRVVEREKRAVGLGAVFLGFLGAAGSTMGAASITLTVQARQL |
| | | LSGIVQQQSNLLRAIEAQQHLLQLTVWGIKQLQARVLAVERYLKD |
| | | QQLLGIWGCSGKLICTTNVPWNSSWSNKSLDEIWDNMTWMQWERE |
| | | ISNYTNIIYRLIEESQNQQEKNEQDLLELDKWASLWNWFDITNWL |
| | | WYIKIFIMIVGGLIGLRIVFAVLSIVNRVRQGYSPLSFQTLTPHP |
| | | RGPDRPERIEEEGGEQGRDRSIRLVSGFLALAWDDLRSLCLFSYH |
| | | RLRDFILIAARTVELLGRSSLKGLRRGWEGLKYLWNLLQYWGQEL |
| | | KNSAISLLDTTAIAVAEWTDRVIEIVQRAGRAILNIPRRIRQGLE |
| | | RALL |
| | | (SEQ ID NO: 119) |

In some embodiments the disclosure provides an immunogenic variant of any one of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118 as disclosed herein. In some embodiments, the variant has at least 90% identity, such as at least 91%, 92%, 93%, 94%, 95%, 96%, 97%, 98%, or 99% identity to SEQ ID NO: 96. In some embodiments, the variant has at least 90% identity, such as at least 91%, 92%, 93%, 94%, 95%, 96%, 97%, 98%, or 99% identity to SEQ ID NO: 97. In some embodiments, the variant has at least 90% identity, such as at least 91%, 92%, 93%, 94%, 95%, 96%, 97%, 98%, or 99% identity to SEQ ID NO: 98. In some embodiments, the variant has at least 90% identity, such as at least 91%, 92%, 93%, 94%, 95%, 96%, 97%, 98%, or 99% identity to SEQ ID NO: 99. In some embodiments, the variant has at least 90% identity, such as at least 91%, 92%, 93%, 94%, 95%, 96%, 97%, 98%, or 99% identity to SEQ ID NO: 100. In some embodiments, the variant has at least 90% identity, such as at least 91%, 92%, 93%, 94%, 95%, 96%, 97%, 98%, or 99% identity to SEQ ID NO: 101. In some embodiments, the variant has at least 90% identity, such as at least 91%, 92%, 93%, 94%, 95%, 96%, 97%, 98%, or 99% identity to SEQ ID NO: 102. In some embodiments, the variant has at least 90% identity, such as at least 91%, 92%, 93%, 94%, 95%, 96%, 97%, 98%, or 99% identity to SEQ ID NO: 103. In some embodiments, the variant has at least 90% identity, such as at least 91%, 92%, 93%, 94%, 95%, 96%, 97%, 98%, or 99% identity to SEQ ID NO: 105. In some embodiments, the variant has at least 90% identity, such as at least 91%, 92%, 93%, 94%, 95%, 96%, 97%, 98%, or 99% identity to SEQ ID NO: 106. In some embodiments, the variant has at least 90% identity, such as at least 91%, 92%, 93%, 94%, 95%, 96%, 97%, 98%, or 99% identity to SEQ ID NO: 108. In some embodiments, the variant has at least 90% identity, such as at least 91%, 92%, 93%, 94%, 95%, 96%, 97%, 98%, or 99% identity to SEQ ID NO: 110. In some embodiments, the variant has at least 90% identity, such as at least 91%, 92%, 93%, 94%, 95%, 96%, 97%, 98%, or 99% identity to SEQ ID NO: 112. In some embodiments, the variant has at least 90% identity, such as at least 91%, 92%, 93%, 94%, 95%, 96%, 97%, 98%, or 99% identity to SEQ ID NO: 114. In some embodiments, the variant has at least 90% identity, such as at least 91%, 92%, 93%, 94%, 95%, 96%, 97%, 98%, or 99% identity to SEQ ID NO: 116. In some embodiments, the variant has at least 90% identity, such as at least 91%, 92%, 93%, 94%, 95%, 96%, 97%, 98%, or 99% identity to SEQ ID NO: 118. Typically, the variant retains the glycan shield and optionally preserve the hypervariable loop anchor sites.

In some embodiments, immunogenic fragments of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118 are provided, including immunogenic fragments comprising one or more redesigned variable loop sequences disclosed in Table 1. In some embodiments, such immunogenic fragments include gp120, an outer domain, V1, V2, and/or V3. Typically, the immunogenic fragment retains the glycan shield and optionally preserve the hypervariable loop anchor sites.

In some embodiments, variants of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118 are provided, including variants that retain immunogenic activity and variants comprising one or more redesigned variable loop sequences disclosed in Table 1. In some embodiments, such variants include gp120, an outer domain, V1, V2, and/or V3. Typically, the variant retains the glycan shield and optionally preserve the hypervariable loop anchor sites.

In some embodiments, the present disclosure provides a polynucleotide comprising a nucleotide sequence that encodes a polypeptide having an amino acid sequence of any one of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment or variant thereof, optionally comprising gp120, an outer domain, V1, V2 and/or V3. Typically, the immunogenic fragment or variant thereof retains the glycan shield and optionally preserve the hypervariable loop anchor sites.

In certain embodiments, the modified consensus sequence or fragment or variant thereof is selected from HIV Env subtype A1, B, C, D, or G, or CRF01_AE or CRF02_AG. In certain embodiments, the modified consensus sequence or fragment or variant thereof is HIV Env subtype M.

In certain embodiments, the modified consensus sequence or immunogenic fragment or variant thereof is from HIV Env subtype A1 and comprises at least one modified HV V1 loop region selected from SEQ ID NO: 33 or SEQ ID NO: 34. In certain embodiments, the modified consensus sequence or immunogenic fragment or variant thereof is from HIV Env subtype B and comprises at least one modified HV V1 loop region selected from SEQ ID NO: 35 or SEQ ID NO: 36. In certain embodiments, the modified consensus sequence or immunogenic fragment or variant thereof is from HIV Env subtype C and comprises at least one modified HV V1 loop region selected from SEQ ID NO: 37 or SEQ ID NO: 38. In certain embodiments, the modified consensus sequence or immunogenic fragment or variant thereof is from HIV Env subtype D and comprises at least one modified HV V1 loop region selected from SEQ ID NO: 39 or SEQ ID NO: 40. In certain embodiments, the modified consensus sequence or immunogenic fragment or variant thereof is from HIV Env subtype G and comprises at least one modified HV V1 loop region selected from SEQ ID NO: 41 or SEQ ID NO: 42. In certain embodiments, the modified consensus sequence or immunogenic fragment or variant thereof is from HIV Env subtype CRF01_AE and comprises at least one modified HV V1 loop region selected from SEQ ID NO: 43 or SEQ ID NO: 44. In certain embodiments, the modified consensus sequence or immunogenic fragment or variant thereof is from HIV Env subtype CRF02_AG and comprises at least one modified HV V1 loop region selected from SEQ ID NO: 45 or SEQ ID NO: 46. In certain embodiments, the modified consensus sequence or immunogenic fragment or variant thereof is from HIV Env subtype M and comprises at least one modified HV V1 loop region selected from SEQ ID NO: 47 or SEQ ID NO: 48.

In certain embodiments, the modified consensus sequence or immunogenic fragment or variant thereof is from HIV Env subtype A1 and comprises at least one modified HV V2 loop region selected from SEQ ID NO: 49 or SEQ ID NO: 50. In certain embodiments, the modified consensus sequence or immunogenic fragment or variant thereof is from HIV Env subtype B and comprises at least one modified HV V2 loop region selected from SEQ ID NO: 51 or SEQ ID NO: 52. In certain embodiments, the modified consensus sequence or immunogenic fragment or variant thereof is from HIV Env subtype C and comprises at least one modified HV V2 loop region selected from SEQ ID NO: 53 or SEQ ID NO: 54. In certain embodiments, the modified consensus sequence or immunogenic fragment or variant thereof is from HIV Env subtype D and comprises at least one modified HV V2 loop region selected from SEQ ID NO: 55 or SEQ ID NO: 56. In certain embodiments, the modified consensus sequence or immunogenic fragment or variant thereof is from HIV Env subtype G and comprises at least one modified HV V2 loop region selected from SEQ ID NO: 57 or SEQ ID NO: 58. In certain embodiments, the modified consensus sequence or immunogenic fragment or variant thereof is from HIV Env subtype CRF01_AE and comprises at least one modified HV V2 loop region selected from SEQ ID NO: 59 or SEQ ID NO: 60. In certain embodiments, the modified consensus sequence or immunogenic fragment or variant thereof is from HIV Env subtype CRF02_AG and comprises at least one modified HV V2 loop region selected from SEQ ID NO: 61 or SEQ ID NO: 62. In certain embodiments, the modified consensus sequence or immunogenic fragment or variant thereof is from HIV Env subtype M and comprises at least one modified HV V2 loop region of SEQ ID NO: 63.

In certain embodiments, the modified consensus sequence or immunogenic fragment or variant thereof is from HIV Env subtype A1 and comprises at least one modified HV V4 loop region selected from SEQ ID NO: 64 or SEQ ID NO: 65. In certain embodiments, the modified consensus sequence or immunogenic fragment or variant thereof is from HIV Env subtype B and comprises at least one modified HV V4 loop region selected from SEQ ID NO: 66 or SEQ ID NO: 67. In certain embodiments, the modified consensus sequence or immunogenic fragment or variant thereof is from HIV Env subtype C and comprises at least one modified HV V4 loop region selected from SEQ ID NO: 68 or SEQ ID NO: 69. In certain embodiments, the modified consensus sequence or immunogenic fragment or variant thereof is from HIV Env subtype D and comprises at least one modified HV V4 loop region selected from SEQ ID NO: 70 or SEQ ID NO: 71. In certain embodiments, the modified consensus sequence or immunogenic fragment or variant thereof is from HIV Env subtype G and comprises at least one modified HV V4 loop region selected from SEQ ID NO: 72 or SEQ ID NO: 73. In certain embodiments, the modified consensus sequence or immunogenic fragment or variant thereof is from HIV Env subtype CRF01_AE and comprises at least one modified HV V4 loop region selected from SEQ ID NO: 74 or SEQ ID NO: 75. In certain embodiments, the modified consensus sequence or immunogenic fragment or variant thereof is from HIV Env subtype CRF02_AG and comprises at least one modified HV V4 loop region selected from SEQ ID NO: 76 or SEQ ID NO: 77. In certain embodiments, the modified consensus sequence or immunogenic fragment or variant thereof is from HIV Env subtype M and comprises at least one modified HV V4 loop region selected from SEQ ID NO: 78 or SEQ ID NO: 79.

In certain embodiments, the modified consensus sequence or immunogenic fragment or variant thereof is from HIV Env subtype A1 and comprises at least one modified HV V5 loop region selected from SEQ ID NO: 80 or SEQ ID NO: 81. In certain embodiments, the modified consensus sequence or immunogenic fragment or variant thereof is from HIV Env subtype B and comprises at least one modified HV V5 loop region selected from SEQ ID NO: 82 or SEQ ID NO: 83. In certain embodiments, the modified consensus sequence or immunogenic fragment or variant thereof is from HIV Env subtype C and comprises at least one modified HV V5 loop region selected from SEQ ID NO: 84 or SEQ ID NO: 85. In certain embodiments, the modified consensus sequence or immunogenic fragment or variant thereof is from HIV Env subtype D and comprises at least one modified HV V5 loop region selected from SEQ ID NO: 86 or SEQ ID NO: 87. In certain embodiments, the modified consensus sequence or immunogenic fragment or variant thereof is from HIV Env subtype G and comprises at least one modified HV V5 loop region selected from SEQ ID NO: 88 or SEQ ID NO: 89. In certain embodiments, the modified consensus sequence or immunogenic fragment or variant thereof is from HIV Env subtype CRF01_AE and comprises at least one modified HV V5 loop region selected from SEQ ID NO: 90 or SEQ ID NO: 91. In certain embodiments, the modified consensus sequence or immunogenic fragment or variant thereof is from HIV Env subtype CRF02_AG and comprises at least one modified HV V5 loop region selected from SEQ ID NO: 92 or SEQ ID NO: 93. In certain embodiments, the modified consensus sequence or immunogenic fragment or variant thereof is from HIV Env subtype M and comprises at least one modified HV V5 loop region selected from SEQ ID NO: 94 or SEQ ID NO: 95.

In certain embodiments, the modified consensus sequence or immunogenic fragment or variant thereof is from HIV Env subtype A1 and comprises a HV V1 loop region of SEQ ID NO: 33, a HV V2 loop region of SEQ ID NO: 49, a HV V4 loop region of SEQ ID NO: 65, and a HV V5 loop region of SEQ ID NO: 81. In certain embodiments, the modified consensus sequence is SEQ ID NO: 96, or an immunogenic fragment or variant thereof. In certain embodiments, the modified consensus sequence or immunogenic fragment or variant thereof is from HIV Env subtype A1 and comprises a HV V1 loop region of SEQ ID NO: 34, a HV V2 loop region of SEQ ID NO: 50, a HV V4 loop region of SEQ ID NO: 64, and a HV V5 loop region of SEQ ID NO: 80. In certain embodiments, the modified consensus sequence is SEQ ID NO: 112, or an immunogenic fragment or variant thereof.

In certain embodiments, the modified consensus sequence or immunogenic fragment or variant thereof is from HIV Env subtype B and comprises a HV V1 loop region of SEQ ID NO: 35, a HV V2 loop region of SEQ ID NO: 51, a HV V4 loop region of SEQ ID NO: 67, and a HV V5 loop region of SEQ ID NO: 83. In certain embodiments, the modified consensus sequence is SEQ ID NO: 97, or an immunogenic fragment or variant thereof. In certain embodiments, the modified consensus sequence or immunogenic fragment or variant thereof is from HIV Env subtype B and comprises a HV V1 loop region of SEQ ID NO: 36, a HV V2 loop region of SEQ ID NO: 52, a HV V4 loop region of SEQ ID NO: 66, and a HV V5 loop region of SEQ ID NO: 82. In certain embodiments, the modified consensus sequence is SEQ ID NO: 116, or an immunogenic fragment or variant thereof. In certain embodiments, the modified consensus sequence or immunogenic fragment or variant thereof is from HIV Env subtype B and comprises a HV V3 loop region of SEQ ID NO: 120.

In certain embodiments, the modified consensus sequence or immunogenic fragment or variant thereof is from HIV Env subtype C and comprises a HV V1 loop region of SEQ ID NO: 37, a HV V2 loop region of SEQ ID NO: 53, a HV V4 loop region of SEQ ID NO: 69, and a HV V5 loop region of SEQ ID NO: 85. In certain embodiments, the modified consensus sequence is SEQ ID NO: 98, or an immunogenic fragment or variant thereof. In certain embodiments, the modified consensus sequence or immunogenic fragment or variant thereof is from HIV Env subtype C and comprises a HV V1 loop region of SEQ ID NO: 38, a HV V2 loop region of SEQ ID NO: 54, a HV V4 loop region of SEQ ID NO: 68, and a HV V5 loop region of SEQ ID NO: 84. In certain embodiments, the modified consensus sequence is SEQ ID NO: 108, or an immunogenic fragment or variant thereof.

In certain embodiments, the modified consensus sequence or immunogenic fragment or variant thereof is from HIV Env subtype D and comprises a HV V1 loop region of SEQ ID NO: 39, a HV V2 loop region of SEQ ID NO: 55, a HV V4 loop region of SEQ ID NO: 71, and a HV V5 loop region of SEQ ID NO: 87. In certain embodiments, the modified consensus sequence is SEQ ID NO: 99, or an immunogenic fragment or variant thereof. In certain embodiments, the modified consensus sequence or immunogenic fragment or variant thereof is from HIV Env subtype D and comprises a HV V1 loop region of SEQ ID NO: 40, a HV V2 loop region of SEQ ID NO: 56, a HV V4 loop region of SEQ ID NO: 70, and a HV V5 loop region of SEQ ID NO: 86. In certain embodiments, the modified consensus sequence is SEQ ID NO: 106, or an immunogenic fragment or variant thereof.

In certain embodiments, the modified consensus sequence or immunogenic fragment or variant thereof is from HIV Env subtype G and comprises a HV V1 loop region of SEQ ID NO: 41, a HV V2 loop region of SEQ ID NO: 57, a HV V4 loop region of SEQ ID NO: 73, and a HV V5 loop region of SEQ ID NO: 89. In certain embodiments, the modified consensus sequence is SEQ ID NO: 100, or an immunogenic fragment or variant thereof. In certain embodiments, the modified consensus sequence or immunogenic fragment or variant thereof is from HIV Env subtype G and comprises a HV V1 loop region of SEQ ID NO: 42, a HV V2 loop region of SEQ ID NO: 58, a HV V4 loop region of SEQ ID NO: 72, and a HV V5 loop region of SEQ ID NO: 88. In certain embodiments, the modified consensus sequence is SEQ ID NO: 114, or an immunogenic fragment or variant thereof.

In certain embodiments, the modified consensus sequence or immunogenic fragment or variant thereof is from HIV Env subtype CRF01_AE and comprises a HV V1 loop region of SEQ ID NO: 43, a HV V2 loop region of SEQ ID NO: 59, a HV V4 loop region of SEQ ID NO: 75, and a HV V5 loop region of SEQ ID NO: 91. In certain embodiments, the modified consensus sequence is SEQ ID NO: 101, or an immunogenic fragment or variant thereof. In certain embodiments, the modified consensus sequence or immunogenic fragment or variant thereof is from HIV Env subtype CRF01_AE and comprises a HV V1 loop region of SEQ ID NO: 44, a HV V2 loop region of SEQ ID NO: 60, a HV V4 loop region of SEQ ID NO: 74, and a HV V5 loop region of SEQ ID NO: 90. In certain embodiments, the modified consensus sequence is SEQ ID NO: 105, or an immunogenic fragment or variant thereof. In certain embodiments, the modified consensus sequence is SEQ ID NO: 104, or an immunogenic fragment or variant thereof.

In certain embodiments, the modified consensus sequence or immunogenic fragment or variant thereof is from HIV Env subtype CRF02_AG and comprises a HV V1 loop region of SEQ ID NO: 45, a HV V2 loop region of SEQ ID NO: 61, a HV V4 loop region of SEQ ID NO: 77, and a HV V5 loop region of SEQ ID NO: 93. In certain embodiments, the modified consensus sequence is SEQ ID NO: 102, or an immunogenic fragment or variant thereof. In certain embodiments, the modified consensus sequence or immunogenic fragment or variant thereof is from HIV Env subtype CRF02_AG and comprises a HV V1 loop region of SEQ ID NO: 46, a HV V2 loop region of SEQ ID NO: 62, a HV V4 loop region of SEQ ID NO: 76, and a HV V5 loop region of SEQ ID NO: 92. In certain embodiments, the modified consensus sequence is SEQ ID NO: 110, or an immunogenic fragment or variant thereof.

In certain embodiments, the modified consensus sequence or immunogenic fragment or variant thereof is from HIV Env subtype M and comprises a HV V1 loop region of SEQ ID NO: 47, a HV V2 loop region of SEQ ID NO: 63, a HV V4 loop region of SEQ ID NO: 79, and a HV V5 loop region of SEQ ID NO: 95. In certain embodiments, the modified consensus sequence is SEQ ID NO: 103, or an immunogenic fragment or variant thereof. In certain embodiments, the modified consensus sequence or immunogenic fragment or variant thereof is from HIV Env subtype M and comprises a HV V1 loop region of SEQ ID NO: 48, a HV V2 loop region of SEQ ID NO: 64, a HV V4 loop region of SEQ ID NO: 78, and a HV V5 loop region of SEQ ID NO: 94. In certain embodiments, the modified consensus sequence is SEQ ID NO: 118, or an immunogenic fragment or variant thereof.

### Vectors and Host Cells

In another aspect, the present disclosure is directed to a vector comprising an isolated polynucleotide comprising a nucleic acid molecule encoding any of the modified Env consensus sequences or immunogenic fragments or variants thereof disclosed herein, or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule that encodes modified Env consensus sequence or immunogenic fragment or variant thereof as disclosed herein.

Generally, any system or vector suitable to maintain, propagate or express a polypeptide in a host may be used for expression of the modified Env consensus sequence or immunogenic fragment or variant thereof as disclosed herein. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001).

In another aspect, the present disclosure is directed to a host cell comprising any of the vectors disclosed herein including the expression vectors comprising the polynucleotide sequences encoding the modified Env consensus sequence or immunogenic fragment or variant thereof as disclosed herein. A wide variety of host cells are useful in expressing the present sequences. Non-limiting examples of suitable host cells include well-known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSCl, BSC40, and BMT10), insect cells (e.g., Sf9), and human cells such as Expi293F cells and HOS cells.

Efficient expression of the present modified Env consensus sequences or immunogenic fragments or variants thereof as disclosed herein depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the sequences of the present disclosure, one skilled in the art will be able to select the proper vectors, expression control sequences, and hosts without undue experimentation to accomplish the desired expression without departing from the scope of this disclosure.

### Immunogenic Compositions and Kits

Further disclosed herein are immunogenic compositions or vaccines comprising the modified Env consensus sequences or immunogenic fragments or variants of the present disclosure. The immunogenic compositions or vaccines can take any suitable form, including the form of solutions, suspensions, emulsion, tablets, pills, pellets, capsules, capsules containing liquids, powders, and sustained-release formulations, injectants, and combinations thereof.

In certain embodiments disclosed herein, the immunogenic compositions or vaccines may comprise a single modified consensus sequence as disclosed herein, such as one of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment or a variant thereof. Further disclosed herein is an immunogenic composition or vaccine comprising a mixture of at least two different modified consensus sequences or immunogenic fragments or variants as disclosed herein, such as a mixture of at least 2, such as at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or immunogenic fragments or variants thereof.

The immunogenic compositions or vaccines disclosed herein comprising modified consensus sequences or immunogenic fragments or variants thereof can be administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The pharmaceutical compositions may be administered parenterally, when possible, at the target cell site, or intravenously. Intravenous or subcutaneous administration of the modified consensus sequences or fragments or variants thereof is preferred in certain embodiments. The immunogenic compositions or vaccines disclosed herein are administered to a patient or subject systemically, parenterally, or locally.

For parenteral administration, the modified consensus sequences or immunogenic fragments or variants thereof can be formulated in a unit dosage injectable form (e.g., solution, suspension, or emulsion) in association with a pharmaceutically acceptable carrier.

Suitable pharmaceutically acceptable carriers include, but are not limited to, nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG). The compositions disclosed herein may be chosen from any suitable form, including injectable suspensions, solutions, sprays, lyophilized powders, syrups, and elixirs. Additional examples of carriers include water, saline, Ringer's solution, dextrose solution, and 5% human serum albumin. Nonaqueous carriers such as fixed oils and ethyl oleate and liposomes may also be used. The carriers disclosed herein may contain minor amounts of additives such as substances that enhance isotonicity and chemical stability, e.g., buffers and preservatives. The modified consensus sequences or fragments or variants thereof may be formulated in such carriers at concentrations of, for example, about 1 mg/ml to 10 mg/ml.

The vaccine or immunogenic composition disclosed herein may contain an adjuvant. As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO4), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity.

Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, J. Immunol., 2009, 183:6186-6197), IL-2, RANTES, GM-CSF, TNF-α, IFN-γ, G-CSF, LFA-3, CD72, B7-1, B7-2, OX-40L and 41 BBL.

The dose and dosage regimen may depend upon a variety of factors, such as the nature of the infection and the characteristics of the particular sequence or sequences to be administered, e.g., its therapeutic index, the patient, and the patient's history. Generally, a amount of at least one modified consensus sequence or immunogenic fragment or variant thereof is administered to a patient in an amount to induce an immune response in the patient. In particular embodiments, the amount of the modified consensus sequence or immunogenic fragment or variant thereof administered is in the range of about 0.1 mg/kg to about 20 mg/kg of patient body weight. Depending on the type and severity of the infection, about 0.1 mg/kg to about 20 mg/kg body weight (e.g., about 0.1-15 mg/kg/dose) of antigen is an initial candidate dosage for administration to the patient, whether, for example, by one or more separate administrations, or by continuous infusion. The progress of the therapy may be readily monitored by conventional methods and assays and based on criteria known to the physician or other persons of skill in the art.

In yet another embodiment, there is provided a kit for performing diagnostic and prognostic assays using the modified consensus sequences or immunogenic fragments or variants disclosed herein. Kits may include a suitable container comprising at least one modified consensus sequence or immunogenic fragment or variant thereof in either labeled or unlabeled form. In addition, when the modified consensus sequences or immunogenic fragments or variants are supplied in a labeled form suitable for an indirect binding assay, the kit further includes reagents for performing the appropriate indirect assay. For example, the kit includes one or more suitable containers including enzyme substrates or derivatizing agents, depending on the nature of the label. Control samples and/or instructions may also be included.

### Methods

In certain embodiments of all aspects of the disclosure, the modified consensus sequences or immunogenic fragments or variants thereof disclosed herein are used in a method of detecting binding of HIV-1 antibodies to antigens, such as Env. Such methods may include, but are not limited to, antigen-binding assays that are well-known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry. In certain embodiments disclosed herein, the binding capacity of the antibodies to the modified consensus Env may be evaluated by cell-based ELISA.

In certain embodiments, disclosed herein is a method of inducing an immune response comprising administering to a subject a vaccine or immunogenic composition comprising at least one modified consensus sequence or immunogenic fragment or variant thereof as disclosed herein. Also disclosed is a vaccine or immunogenic composition as disclosed herein for use in a method of inducing an immune response in a subject. Also disclosed is an immunogenic composition as disclosed herein for the manufacture of a vaccine for use in a method of inducing an immune response in a subject. The immune response can be induced in a naïve subject who has not previously been exposed to HIV or in a subject who is as risk for HIV-related disorders. Alternatively, the immune response can be induced in a subject who has been previously exposed to HIV and used to enhance an existing immune response.

In one embodiment, the method of inducing an immune response (or related uses thereof) comprises administering to a subject a pharmaceutical composition comprising at least one, such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or immunogenic fragments or variants thereof.

In the methods or uses disclosed herein, the immune response can be measured using routing methods in the art, such as those disclosed in this application. These routine methods include, but are not limited to, measuring antibody response, such as an antibody response directed against an HIV-1 Env protein.

In certain embodiments, there are further disclosed herein methods of treating or preventing HIV infection comprising administering to a subject vaccine or immunogenic composition comprising at least one modified consensus sequence or immunogenic fragment or variant thereof as disclosed herein. Also disclosed is a vaccine or immunogenic composition as disclosed herein for use in a method of treating or preventing HIV infection in a subject. Also disclosed is an immunogenic composition as disclosed herein for the manufacture of a vaccine for use in a method of treating or preventing HIV infection in a subject.

In one embodiment, the method of treating or preventing HIV infection (or related uses thereof) comprises administering to a subject a pharmaceutical composition comprising at least one, such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 of SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or immunogenic fragments or variants thereof.

Subjects at risk for HIV-related diseases or disorders include patients who have come into contact with an infected person or who have been exposed to HIV in some way. Administration of a immunogenic composition or vaccine comprising at least one modified consensus sequence or immunogenic fragment or variant thereof as disclosed herein can occur prior to the manifestation of symptoms characteristic of HIV-related disease or disorder, such that a disease or disorder is prevented or delayed in its progression.

In certain aspects, the methods of treating or preventing HIV infection (or related uses thereof) may further comprise co-administering other agents suitable for the treatment of HIV infection, such as anti-retroviral therapies. Anti-retroviral therapies that may be co-administered with the modified consensus sequences or fragments or variants thereof disclosed herein may include, for example, nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors (such as bevirimat and vivecon), or broad spectrum inhibitors, such as natural antivirals.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Methods for Hypervariable Loop Analysis

The HXB2 genome annotation from LANL HIV Database, available online on the world wide web at hiv.lanl.gov, was followed to define the start and end of HV loops, which were located at 132-152, 185-190, 396-410 and 460-467, for V1HV, V2HV, V4HV and V5HV, respectively. As an exception, V4HV was extended to include 395 and 411-412 anchor sites. Structurally, the distance between the start and end of spacers was calculated as the distance between CA atoms before the N-terminal and after the C-terminal of the spacer loop. The HXB2 sequence was added and aligned to define the positions. Multiple Alignment using Fast Fourier Transform (MAFFT) was used to align sequences. For the logo plot of HVs, HV loops were split from the middle. The first half was left aligned to the start position while the second half was right aligned to the end position.

Structure Prediction and Analysis: ColabFold, which integrated AlphaFold-Multimer with other tools and databases, was used to model unmodified and redesigned Env structures. During the prediction, one of the three subunits in the prefusion Env trimer was predicted by feeding one gp120 (31-511) and one gp41 (512-665) as input. The top model from five predictions was adopted for later analysis.

The non-HV surrounding surface sites of HV loops were defined as Env sites that were not part of the HV loop which had a relative accessible surface area (RSA) greater or equal to 0.30 and had any atoms within 10 Å of any HV loop atoms in the predicted unmodified consensus B Env structure. The accessible surface area (ASA) was approximated by the Shrake-Rupley method. In brief, evenly distributed points (n=256) were placed on spheres that were centered at atoms in the protein. The radius of the spheres was the sum of the atoms' and the solvent probe's radius (1.5 Å). The accessible surface was defined after filtering out points on a sphere that were within neighboring spheres. Then, the RSA of a site was calculated as the ratio of ASA over the estimated maximum ASA. The same approach was also used to estimate depth, only the probe radius was set to 30 Å, and the depth was equal to the minimal distance between a protein atom and the accessible surface minus the probe radius. Env structure 5FYJ (Stewart-Jones, G. B. E. et al. Trimeric HIV-1-Env Structures Define Glycan Shields from Clades A, B, and G. Cell 165, 813-826 (2016)) was used for the ASA estimation. It was also used for the illustration of HV loop and nearby bnAb epitopes.

The collision between antibody and Env was determined as the number of antibody atoms within 2.5 Å of Env after superimposing the modeled Env on the Env in the solved Env-antibody complex. Bio.PDB (Hamelryck, T. & Manderick, B. PDB file parser and structure class implemented in Python. Bioinforma. Oxf. Engl. 19, 2308-2310 (2003)) was used to superimpose structures. The Env-antibody complexes used, listed as PDB code, were 5V8L, 6VTT, 6UDJ, 4JM2, 6V8X and 7PC2 for PGT145, VRC26.25, 10-1074, PGT135, VRC01 and 3BNC117, respectively. Lee, J. H. et al. A Broadly Neutralizing Antibody Targets the Dynamic HIV Envelope Trimer Apex via a Long, Rigidified, and Anionic β-Hairpin Structure. Immunity 46, 690-702 (2017); Gorman, J. et al. Structure of Super-Potent Antibody CAP256-VRC26.25 in Complex with HIV-1 Envelope Reveals a Combined Mode of Trimer-Apex Recognition. Cell Rep. 31, 107488 (2020).; Schommers, P. et al. Restriction of HIV-1 Escape by a Highly Broad and Potent Neutralizing Antibody. Cell 180, 471-489.e22 (2020); Kong, L. et al. Supersite of immune vulnerability on the glycosylated face of HIV-1 envelope glycoprotein gp120. Nat. Struct. Mol. Biol. 20, 796-803 (2013); Henderson, R. et al. Disruption of the HIV-1 Envelope allosteric network blocks CD4-induced rearrangements. Nat. Commun. 11, 520 (2020); and Lorin, V. et al. Epitope convergence of broadly HIV-1 neutralizing IgA and IgG antibody lineages in a viremic controller. J. Exp. Med. 219, e20212045 (2022). PyMol (Schrödinger, LLC. The PyMOL Molecular Graphics System, Version 1.8. (2015)) was used for the structure visualization and figure generation. Hydrogen atoms were removed from the structure, if there was any, in all analysis.

### Example 1—Analysis of Short HV Loops for HIV-1 Subtypes B, C, and CRF01_AE

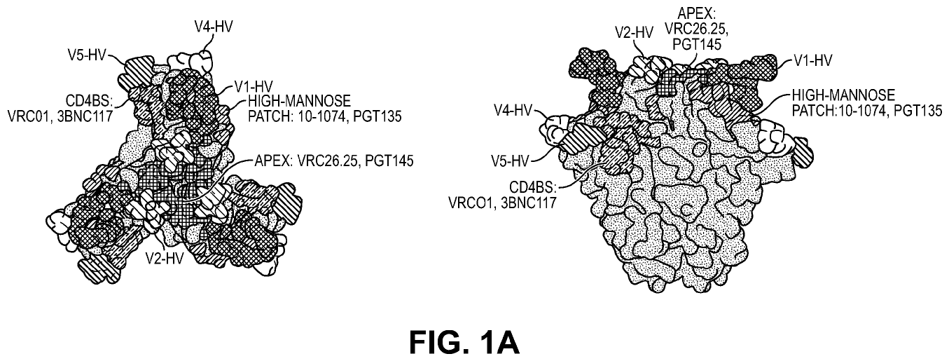

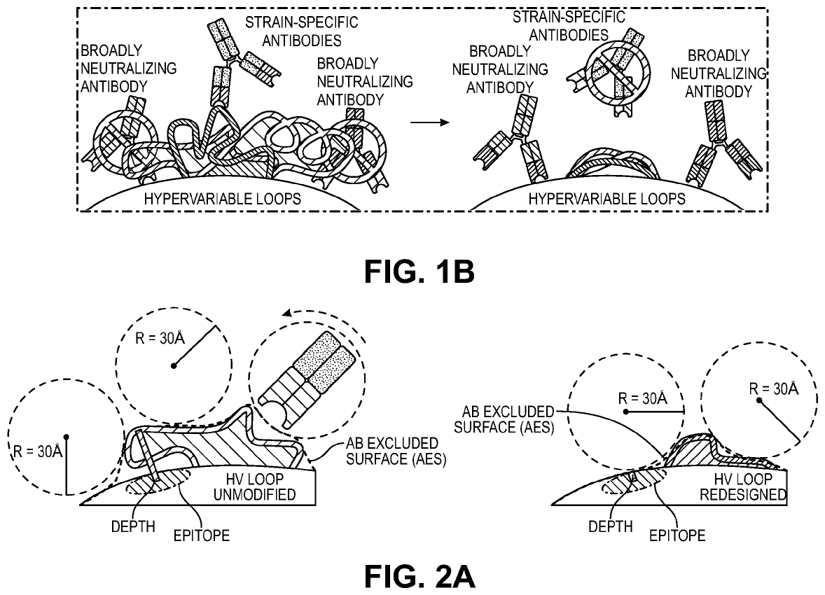

Short HV Loops Were Rare for Subtypes B, C, and CRF01_AE: publicly available circulating HIV-1 sequences were curated to obtain a dataset of 4,847 independent Env sequences sampled from people living with HIV between 1979 and 2020. These sequences corresponded to HIV-1 subtype B (n=2495), subtype C (n=1503) and CRF01_AE (n=849). The distribution of HV loop lengths was analyzed as well as the number of potential N-linked glycosylation sites (PNGS) in four HV loops. The HV loops corresponded to a portion of the variable loops: V1HV, sites 132-152 within sites 131-157 (V1); V2HV, sites 185-190 within sites 157-196 (V2); V4HV, sites 395-412 within sites 385-418 (V4); and V5HV, sites 460-467 within sites 460-470 (V5). V3 was omitted because it does not contain an HV segment. The location of the HV loops, as well as adjacent bnAb epitopes, are shown on the structure in FIG. 1A.

V1HV loops showed a median length of 20 (subtype C) to 22 (CRF01_AE) amino acids. V1HV loops were about twice as long as other HV loops, which had a median length ranging between 7 amino acids (V2HV) and 12 amino acids (V4HV). The length distributions across subtypes/CRF were similar for V2HV and V5HV, while there were differences for V1HV and V4HV. CRF01_AE showed fewer short V1HV loops (25th percentile=22 amino acids), than subtype B (19 amino acids) or subtype C (16 amino acids). Subtype B had fewer short V4HV loops (25th percentile=11 amino acids) than subtype C (6 amino acids) and CRF01_AE (7 amino acids). Similar patterns were observed for the number of PNGS on HV loops across subtypes. Sequences with V1, V4, and V5 HV loops that were shorter than or equal to the 10th percentile were very rare representing 0.73%, 0.53%, and 0.32% of the dataset for subtypes B, C, and CRF01_AE, respectively. The loop lengths of the sequences that have been used as inserts in previous vaccine efficacy or are considered for future trials were also determined. It was found that these insert sequences usually had one or more long HV-loops. For example, the TV1 sequence used in the vaccine in HVTN100 and HVTN702 efficacy trial had a 33 amino acid long V1HV, which is about three times longer than the length of the redesigned V1HVs (8 to 13 amino acids). Bekker, L.-G. et al. Subtype C ALVAC-HIV and bivalent subtype C gp120/MF59 HIV-1 vaccine in low-risk, HIV-uninfected, South African adults: a phase ½ trial. Lancet HIV 5, e366-e378 (2018); and Gray, G. E. et al. Vaccine Efficacy of ALVAC-HIV and Bivalent Subtype C gp120-MF59 in Adults. N. Engl. J. Med. 384, 1089-1100 (2021). Mosaic2.Env in the Mosaico study (Barouch, D. H. et al. Mosaic HIV-1 vaccines expand the breadth and depth of cellular immune responses in rhesus monkeys. Nat. Med. 16, 319-323 (2010)) had a 16 amino acid V2HV that is three times longer than the designed V2HVs (4 to 5 amino acids).

### Example 2—Analysis of Long HV Loops and Sensitivity to bnAbs

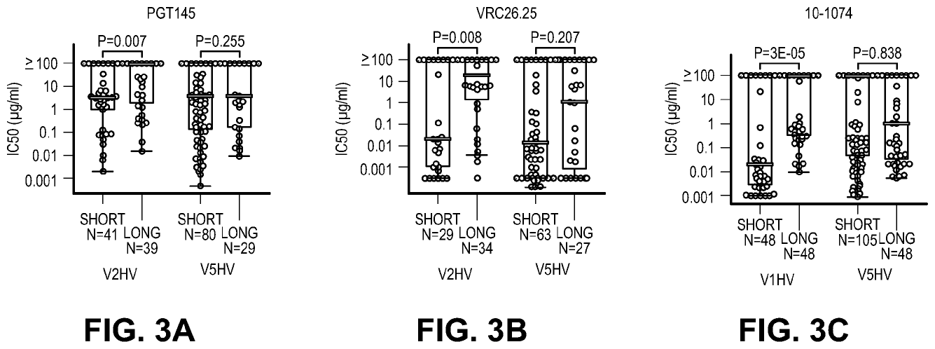

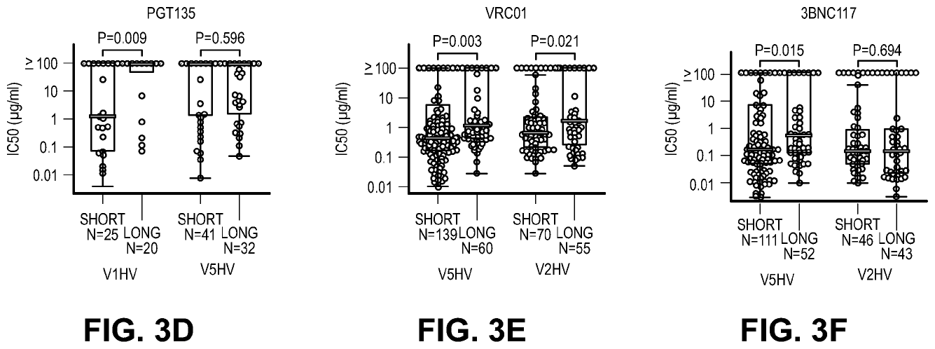

The sensitivity to six bnAbs was compared for HIV-1 sequences with the shortest (≤5 percentile) and longest (≥95 percentile) HV loops. The bnAbs belonged to three representative epitope groups: (1) V2-apex antibodies PGT145 and VRC26.25; (2) glycan-supersite antibodies 10-1074 and PGT135; and (3) CD4bs antibodies VRC01 and 3BNC117. To evaluate the effect of loop length on bnAb sensitivity, the cognate HV loops corresponding to each bnAb were analyzed, and a distal HV loop was used as a control for each comparison. All neutralization and corresponding sequence data were obtained from CATNAP (Yoon, H. et al. CATNAP: a tool to compile, analyze and tally neutralizing antibody panels. Nucleic Acids Res. 43, W213-219 (2015)). As shown in FIGS. 3A-F, the virus strains with the longest cognate HV loops had significantly higher half maximal inhibitory concentration (IC₅₀) than the sequences with the shortest HV loops (p-value≤0.015, one-tailed Mann-Whitney U test) for the six bnAbs. When HV loops distant from the bnAb epitope were compared, there was no significant difference in sensitivity for short or long HV loops for five of the six bnAbs tested (p-value≥0.207). The results for PGT145 are shown in FIG. 3A, VRC26.25 are shown in FIG. 3B; 10-1074 are shown in FIG. 3C, and PGT135 are shown in FIG. 3D. The results for VRC01 are shown in FIG. 3E, and the results for 3BNC117 are shown in FIG. 3F. For VRC01, sequences with shorter HV loops for both V2 and V5 were associated with increased sensitivity when compared to sequences with longer HV loops, as shown in FIG. 3E. An exemplary graphical illustration of interference from long HV loops on the binding potential of bnAbs that target adjacent sites, as well as the improvement in accessibility afforded by short HV loops, is shown, for example, in FIG. 1B.

The strongest impact was seen for V1HV loops and the neutralization sensitivity to 10-1074. HIV-1 Envs with the shortest V1HV loops had a median IC₅₀ greater than or equal to 5,000 times lower than the strains with the longest V1 HV loops (0.02 μg/ml vs. ≥100 μg/ml, one-tailed Mann-Whitney U test p-value 0.00003). For V5 HV loops, which are distant from the 10-1074 epitope, there was no significant difference in sensitivity when short and long loops were compared (one-tailed Mann-Whitney U test p-value 0.207). These data masked some subtype-specific differences, as differences between short and long HV loops were not manifest for subtype B except for 10-1074. Differences for subtype C were generally statistically significant, except for 10-1074 (p=0.129) and PGT135 (p=0.128). Overall, these results indicated that a long HV loop can diminish the virus's sensitivity to a bnAb.

### Example 3—Redesign of HV Loops

The HV-loops were redesigned to improve accessibility to typical bnAb epitopes. The redesign was done for subtypes B, C, and CRF01_AE. These subtypes/were selected in part because large datasets of sequences were available. In addition, HIV-1 subtype B is the most thoroughly characterized subtype, subtype C is responsible for the majority of cases among people living with HIV, and CRF01_AE is the virus that was circulating in a HIV-1 vaccine trial where some efficacy was recorded. The goals of this study were to: (1) make the loop as short as possible while maintaining structural integrity; (2) preserve the loop anchor sites, which are the relatively conserved start and end of the HV loops that form favorable interactions with the non-HV part of Env; and (3) maintain the glycan shield intact by ensuring there was at least one PNGS on each redesigned loop. To apply this strategy to the V1HV loop of subtype B, the sequence alignment and the AlphaFold2 predicted structure of the corresponding consensus sequence were analyzed. We identified the relatively conserved TDL motif (sites 132-134) as N-anchor sites was identified, as well as the relatively conserved MEKG (SEQ ID NO: 121) motif (sites 149-152) as C-anchor sites, with all sites in between considered spacer sites. In the TDL motif, D133 may form favorable charged interactions with K151 in the C-anchor sites or with K154; L134 may form hydrophobic interactions with I154, L175, I323, and I326. In the MEKG (SEQ ID NO: 121) motif, M149 may form hydrophobic interactions with I326 and I154; E150 might form a favorable charged interaction with R419. These anchor sites were retained since they did not interfere with the binding of glycan supersite antibodies. Different spacer lengths were tested iteratively using AlphaFold2 predicted structures for each length, and the 15 amino acid spacer was shortened to 5 amino acids. This resulted in the V1HV loop length changing from 22 amino acids (50^{th} percentile in subtype B sequences) to 12 amino acids (4^{th} percentile in subtype B sequences). In addition, for subtype B, V2HV was modified from 7 to 4 amino acids, and V5HV was modified from 7 to 5 amino acids. The V1HV, V2HV, and V5HV loops of subtypes B, C, and CRF01_AE were redesigned with the same strategy, and the corresponding redesigned hypervariable loop sequences are shown in Table 1.

Contrary to the strategy for V1HV, V2HV, and V5HV loops, for V4HV loops, the shortest V4HV loops to accommodate its specific structural context was not sought. Sites adjacent to V4HV (non-HV sites within 10 Å of the HV loops, relative surface area>0.30) were diverse, and no major bnAbs are known to target this segment of Env. For subtype C, the median Shannon entropy of V4HV adjacent sites was 2.4 bits compared to 0.8, 0.7, and 1.1 bits, respectively, for the sites adjacent to V1HV, V2HV and V5HV. This high Shannon entropy for V4HV loops indicated that there were 4.5 (2^{2.4/1.1}) to 10.8 (2^{2.4/0.7}) times more amino acid possibilities on average for surface sites adjacent to V4HV compared to the other HV loops. In addition, the distance encompassing spacer sites was larger for V4HV than for V1HV, V2HV, and V5HVs (20.8 Å for V4HV, versus 10.8, 7.9, and 9.4 Å for V1HV, V2HV and V5HV, respectively, based on the AlphaFold2 model of the subtype B consensus). This suggests that a short V4HV spacer may not be compatible with the structure. Hence, a medium length was chosen for the V4HV loop; this loop length could also partially shield the highly diverse segment adjacent to the V4HV. For the subtype B consensus, W395 was identified as the N-terminal anchor site, and ND (sites 411-412) was identified as the C-terminal anchor sites. The spacer was replaced with an 11-amino acid linker. The redesigned linker contained two PNGS, which was the median number for V4HV subtype B, C, and CRF01 sequences. Across all redesigned HV loops, the spacer consisted mainly of glycine and serine that lack the side chains that are preferentially targeted by antibodies.

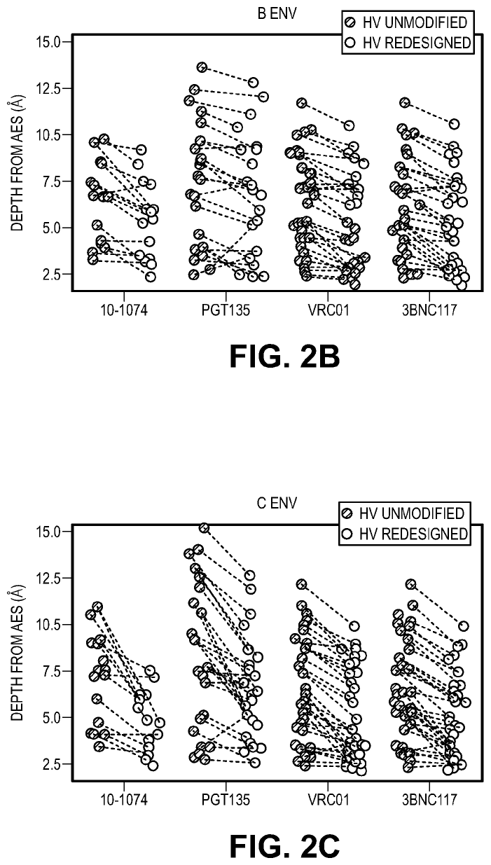

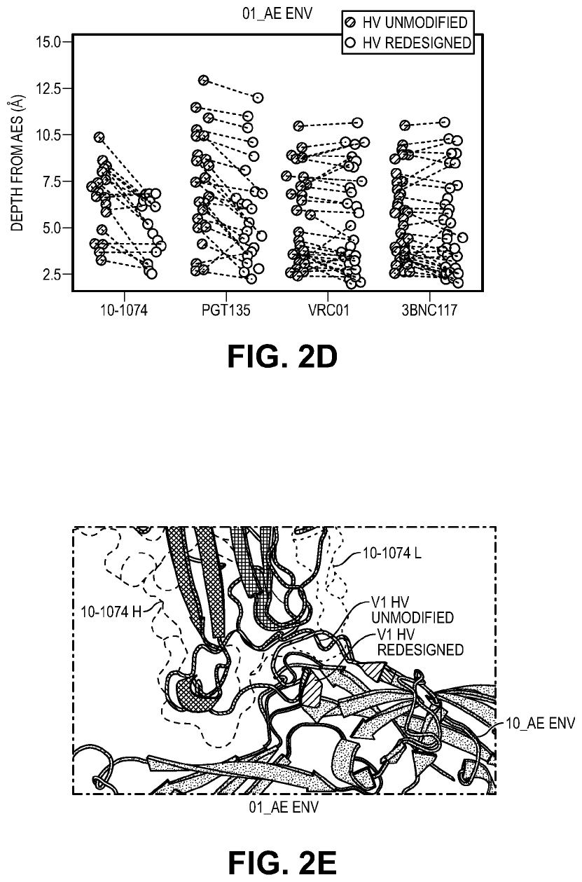

To measure if the redesign improved accessibility bnAb epitopes, a depth index was calculated to correspond to the closest distance between an epitope site and the antibody probe. The antibody probe is a sphere with a radius corresponding to an antibody's Fv (=30 Å) that rolls around the Env structure, as illustrated in FIG. 2A. A short depth indicates that the site is more easily accessible to the antibody. The depth was calculated corresponding to the epitopes of two glycan supersite bnAbs, 10-1074 and PGT135, and two CD4bs bnAbs, VRC01 and 3BNC117, before and after the HV loop redesign. The epitope depth decreased for the four bnAb epitopes based on the structural model of the Env consensus of subtypes B, C, and CRF01 AE, as shown in FIGS. 2B-2D. The median depth decreased by 0.8 to 2.5 Å for glycan supersite antibody epitope sites, while the median depth decreased by 0.1 to 1.8 Å for CD4bs antibody epitope sites. The apex antibodies, PGT145 and VRC26.25, were not tested because their epitopes are quaternary and require modeling of the entire Env trimer, which was beyond the scope of the current endeavor.

Focusing on CRF01_AE, the consensus sequence had a IGNITD (SEQ ID NO: 122) motif (147-152) as its V1HV C-anchor sites. AlphaFold2 predicted that 1147 occupied the hydrophobic pocket formed by R327, K328, Y330, and P417, which corresponds to the spot targeted by F^{100G} on the CDR-H3 of 10-1074, as shown in FIG. 2E. This meant that the 10-1074 epitope was hidden with a median depth of 7.0 Å. After the HV loops redesign, the 24 amino acid-long V1HV loop (58.2 percentile in CRF01_AE) was replaced with a 9 amino acid-long loop (0.6 percentile in CRF01_AE). Hence, the median depth of 10-1074 epitope sites was reduced to 5.3 Å. The redesigned Env reduced the number of collisions with 10-1074. See FIG. 2E. A similar decrease in the number of collisions was observed for another glycan supersite bnAb, PGT135, but the impact was limited for CD4bs antibodies (VRC01 and 3BNC117). The number of collisions with subtypes B, C, and CRF01_AE for antibodies 10-1074, PGT135, VRC01, and 3BBNC117 before and after redesign is shown below in Table 3.

TABLE 3
Antibody atoms colliding with Env for unmodified and redesigned HV loops

| HIV-1 | 10-1075 | | PGT135 | | VRC01 | | 3BNC117 | |
|---|---|---|---|---|---|---|---|---|
| Subtype | Unmod. | Redesign | Unmod. | Redesign | Unmod. | Redesign | Unmod. | Redesign |
| B | 105 | 19 | 103 | 37 | 18 | 16 | 12 | 6 |
| C | 71 | 25 | 91 | 32 | 19 | 16 | 13 | 8 |
| CRF01_AE | 110 | 73 | 105 | 54 | 13 | 18 | 3 | 5 |

Without wishing to be bound by theory, it is postulated that the greater depth and collision reduction for glycan supersite epitope sites near V1HV was likely due to the fact that V1HV loops were longer than other HV loops, and the HV-loop redesign removed more spacer residues than for other HV loops.

### Example 4—Redesigned HV Loops Limit Interference of Glycan Supersite Epitope

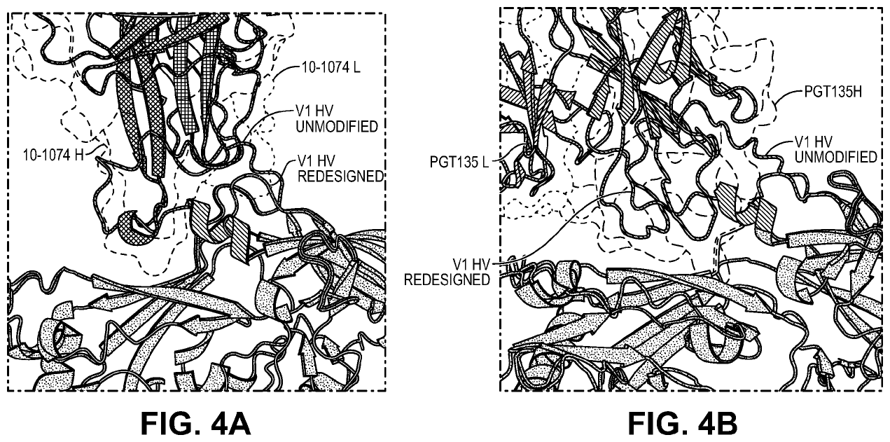

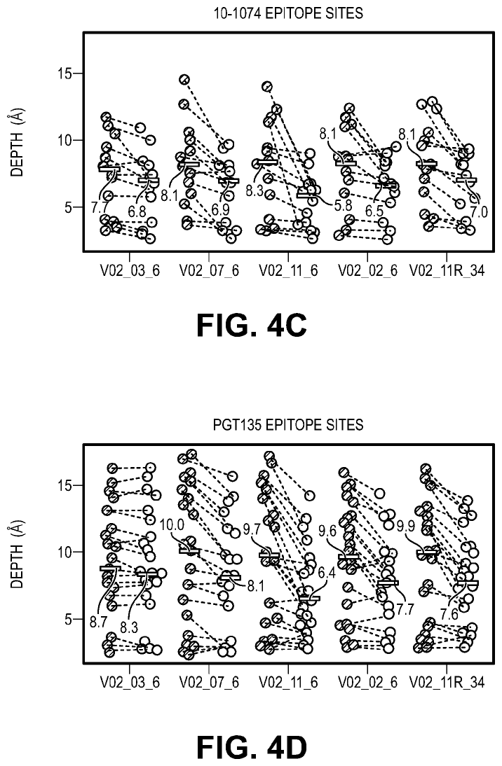

To test if the HV loop redesign can promote bnAbs binding to Env, Env sequences were redesigned as sampled from one person living with HIV, 20337, who was enrolled in Kenya as part of the prospective acute HIV-1 infection RV217 cohort. Robb, M. L. et al. Prospective Study of Acute HIV-1 Infection in Adults in East Africa and Thailand. N. Engl. J. Med. 374, 2120-2130 (2016). The sequences sampled from this participant during acute HIV-1 infection showed that the infection was established with multiple HIV-1 founder variants and developed neutralization breadth a few years after diagnosis. Townsley 2021; and Lewitus, E. et al. HIV-1 infections with multiple founders associate with the development of neutralization breadth. PLOS Pathog. 18, e1010369 (2022). Five Env variants that covered the diversity found in the first month of infection carried long V1HV loops (26 and 30 amino acids), while their glycan supersite epitopes were similar to those in known sensitive viruses in the CATNAP database. The V1HVs were redesigned to make them shorter (9 to 15 amino acids). The redesigned loops reduced the interference from V1HV on the epitopes of two glycan supersite bnAbs, 10-1074 and PGT135, as shown in Table 4 below and as illustrated in FIGS. 4A and 4B.

TABLE 4
Redesigned V1HV Loop of 5 Env Variants from 20337

| | V1HV length | | 10-1074 | | PGT135 | |
|---|---|---|---|---|---|---|
| | (amino acids) | | collisions | | collisions | |
| Variant | Unmod. | Redesign | Unmod. | Redesign | Unmod. | Redesign |
| #1 | 26 | 12 | 100 | 9 | 115 | 25 |
| #2 | 26 | 14 | 92 | 15 | 133 | 34 |
| #3 | 30 | 9 | 88 | 1 | 152 | 12 |
| #4 | 30 | 13 | 73 | 9 | 152 | 26 |
| #5 | 30 | 15 | 101 | 25 | 152 | 34 |

The median depth of the 10-1074 epitope on five Env variants was reduced from 7.7-8.3 Å to 5.8-7.0 Å (FIG. 4C), and for the PGT135 epitope, the median depth was reduced from 8.7-10.0 Å to 6.4-8.3 Å (FIG. 4D). The number of collisions was reduced from 73-101:Env interactions to 1-25 for 10-1074, and from 115-152 to 12-34 for PGT135:Env interactions.

The structural modeling disclosed herein uncovered a novel mechanism for CRF01_AE's resistance to glycan supersite antibodies such as 10-1074. In addition to the absence of PNGS on site 332, 1147 in CRF01_AE V1HV N-anchor sites was predicted to occupy the same location as the CDRH3 of 10-1074. In contrast, PGT128, a V3-glycan antibody whose epitope does not include I147 and is thus less occluded by the CRF01 AE V1HV, retained the capacity to neutralize half of the CRF01_AE viruses. It is possible that other viruses with long V1HV use a similar mechanism to obstruct the binding of glycan supersite bnAbs, highlighting that reducing the length of HV loops could play a key role in minimizing interference with bnAbs targeting adjacent epitopes.

1. A modified HIV-1 Env polypeptide comprising an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof,
wherein the polypeptide comprises at least one modified hypervariable (HV) loop region.

2. The modified HIV-1 Env polypeptide of claim 1, wherein the at least one modified HV loop region is a V1 HV loop region, a V2 HV loop region, a V4 HV loop region, or a V5 HV loop region, and/or wherein the modified HIV-1 Env polypeptide is of a subtype A, B, C, D, G, or M or a circulating recombinant form 01_AE or 02_AG.

3. (canceled)

4. The modified HIV-1 Env polypeptide of claim 1, wherein the at least one modified HV loop region is a V1 HV loop region chosen from SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48.

5. The modified HIV-1 Env polypeptide of claim 1, wherein the at least one modified HV loop region is a V2 HV loop region chosen from SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, or SEQ ID NO: 63.

6. The modified HIV-1 Env polypeptide of claim 1, wherein the at least one modified HV loop region is a V4 HV loop region chosen from SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, or SEQ ID NO: 79.

7. The modified HIV-1 Env polypeptide of claim 1, wherein the at least one modified HV loop region is a V5 HV loop region chosen from SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95.

8. The modified HIV-1 Env polypeptide of claim 1, wherein:
(i) the HIV-1 is subtype A and wherein the modified HIV-1 Env polypeptide comprises:
(a1) a V1 HV loop region chosen from SEQ ID NO: 33 or SEQ ID NO: 34;
(b1) a V2 HV loop region chosen from SEQ ID NO: 49 or SEQ ID NO: 50;
(c1) a V4 HV loop region chosen from SEQ ID NO: 64 or SEQ ID NO: 65; and
(d1) a V5 HV loop region chosen from SEQ ID NO: 80 or SEQ ID NO: 81;
(ii) the HIV-1 is subtype B and wherein the modified HIV-1 Env polypeptide comprises:
(a2) a V1 HV loop region chosen from SEQ ID NO: 35 or SEQ ID NO: 36;
(b2) a V2 HV loop region chosen from SEQ ID NO: 51 or SEQ ID NO: 52;
(c2) a V4 HV loop region chosen from SEQ ID NO: 66 or SEQ ID NO: 67; and
(d2) a V5 HV loop region chosen from SEQ ID NO: 82 or SEQ ID NO: 83;
(iii) the HIV-1 is subtype C and wherein the modified HIV-1 Env polypeptide comprises:
(a3) a V1 HV loop region chosen from SEQ ID NO: 37 or SEQ ID NO: 38;
(b3) a V2 HV loop region chosen from SEQ ID NO: 53 or SEQ ID NO: 54;
(c3) a V4 HV loop region chosen from SEQ ID NO: 68 or SEQ ID NO: 69; and
(d3) a V5 HV loop region chosen from SEQ ID NO: 84 or SEQ ID NO: 85;
(iv) the HIV-1 is subtype D and wherein the modified HIV-1 Env polypeptide comprises:
(a4) a V1 HV loop region chosen from SEQ ID NO: 39 or SEQ ID NO: 40;
(b4) a V2 HV loop region chosen from SEQ ID NO: 55 or SEQ ID NO: 56;
(c4) a V4 HV loop region chosen from SEQ ID NO: 70 or SEQ ID NO: 71; and
(d4) a V5 HV loop region chosen from SEQ ID NO: 86 or SEQ ID NO: 87;
(v) the HIV-1 is subtype G and wherein the modified HIV-1 Env polypeptide comprises:
(a5) a V1 HV loop region chosen from SEQ ID NO: 41 or SEQ ID NO: 42;
(b5) a V2 HV loop region chosen from SEQ ID NO: 57 or SEQ ID NO: 58;
(c5) a V4 HV loop region chosen from SEQ ID NO: 72 or SEQ ID NO: 73; and
(d5) a V5 HV loop region chosen from SEQ ID NO: 88 or SEQ ID NO: 89; or
(vi) the HIV-1 is subtype M and wherein the modified HIV-1 Env polypeptide comprises:
(a6) a V1 HV loop region chosen from SEQ ID NO: 47 or SEQ ID NO: 48;
(b6) a V2 HV loop region is SEQ ID NO: 63;
(c6) a V4 HV loop region chosen from SEQ ID NO: 78 or SEQ ID NO: 79; and
(d6) a V5 HV loop region chosen from SEQ ID NO: 94 or SEQ ID NO: 95.

9-13. (canceled)

14. The modified HIV-1 Env polypeptide of claim 1, wherein:
(i) the HIV-1 is circulating recombinant form 01_AE and wherein the modified HIV-1 Env polypeptide comprises:
(a1) a V1 HV loop region chosen from SEQ ID NO: 43 or SEQ ID NO: 44;
(b1) a V2 HV loop region chosen from SEQ ID NO: 59 or SEQ ID NO: 60;
(c1) a V4 HV loop region chosen from SEQ ID NO: 74 or SEQ ID NO: 75; and
(d1) a V5 HV loop region chosen from SEQ ID NO: 90 or SEQ ID NO: 91 or
(ii) the HIV-1 is circulating recombinant form 02_AG and wherein the modified HIV-1 Env polypeptide comprises:
(a2) a V1 HV loop region chosen from SEQ ID NO: 45 or SEQ ID NO: 46;
(b2) a V2 HV loop region chosen from SEQ ID NO: 61 or SEQ ID NO: 62;
(c2) a V4 HV loop region chosen from SEQ ID NO: 76 or SEQ ID NO: 77; and
(d2) a V5 HV loop region chosen from SEQ ID NO: 92 or SEQ ID NO: 93.

15. (canceled)

16. A vaccine or immunogenic composition for inducing an immune response in a subject against HIV, wherein the vaccine or immunogenic composition comprises at least one modified HIV-1 Env polypeptide according to claim 1, and an optional adjuvant.

17. The vaccine or immunogenic composition of claim 16, comprising at least two modified HIV-1 Env polypeptides.

18. An isolated polynucleotide comprising a nucleotide sequence that encodes a modified HIV-1 Env polypeptide having an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified hypervariable (HV) loop region.

19. The isolated polynucleotide of claim 18, wherein the polynucleotide is a mRNA or a DNA molecule.

20. A method of inducing an immune response against HIV in a subject, the method comprising: administering to the subject the modified HIV-1 Env polypeptide of claim 1, or the immunogenic fragment thereof, a polynucleotide encoding said modified HIV-1 Env polypeptide or immunogenic fragment thereof, or a vaccine or immunogenic composition comprising said modified HIV-1 Env polypeptide or immunogenic fragment thereof or said polynucleotide.

21. A method of inducing an immune response against HIV in a subject, the method comprising: administering to the subject at least one messenger RNA (mRNA) encoding a polypeptide having an amino acid sequence having at least 90% sequence identity with SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 108, SEQ ID NO: 110, SEQ ID NO: 112, SEQ ID NO: 114, SEQ ID NO: 116, or SEQ ID NO: 118, or an immunogenic fragment thereof, wherein the polypeptide comprises at least one modified hypervariable (HV) loop region, and a pharmaceutically acceptable carrier.

22. The method of claim 20, wherein the subject is a human.

23. The method of claim 20, further comprising administering at least one adjuvant to the subject.

24. A method of identifying an antibody against HIV in a sample, comprising:
(1) contacting the sample with at least one modified HIV-1 Env polypeptide according to claim 1, and
(2) determining whether a complex forms between the antibody and the at least one polypeptide or immunogenic fragment thereof, thereby identifying the antibody against HIV in the sample.

25. The method of claim 24, wherein the sample is a tissue sample or a body fluid sample.

26. The method of claim 25, wherein the body fluid sample is selected from blood, plasma, serum, saliva, tear, urine, cerebrospinal fluid, pleural effusion, ascites, and/or peritoneal effusion.

27. The method of claim 24, wherein the antibody is chosen from PGT145, VRC26.25, 10-1074, PGT135, VRC01 or 3BNC117.

28-29. (canceled)


SEQUENCE LISTING (150 sequences)

SEQ ID NO: 1 - protein, 21 aa, Human immunodeficiency virus 1
SNNNTTNTNNTNSTVTGEMRE

SEQ ID NO: 2 - protein, 22 aa, Human immunodeficiency virus 1
TDLNNTNTNNTNSSNSSTMEKG

SEQ ID NO: 3 - protein, 20 aa, Human immunodeficiency virus 1
TNVNNTNTNTNNNTNNNIKE

SEQ ID NO: 4 - protein, 19 aa, Human immunodeficiency virus 1
TDWKNNNTTNNNTTTSEDI

SEQ ID NO: 5 - protein, 23 aa, Human immunodeficiency virus 1
TNVTNTNNTNNTNNNTTTVDGKE

SEQ ID NO: 6 - protein, 24 aa, Human immunodeficiency virus 1
TNANLTNTNTTNNTNTTNIGNITD

SEQ ID NO: 7 - protein, 19 aa, Human immunodeficiency virus 1
SNANNSTSNNRNITNEMKG

SEQ ID NO: 8 - protein, 22 aa, Human immunodeficiency virus 1
TNANNTNTNNTNNTNTTTEEKE

SEQ ID NO: 9 - protein, 8 aa, Human immunodeficiency virus 1
NENNNSSE

SEQ ID NO: 10 - protein, 7 aa, Human immunodeficiency virus 1
DNNNNTS

SEQ ID NO: 11 - protein, 8 aa, Human immunodeficiency virus 1
DNNNNSSE

SEQ ID NO: 12 - protein, 7 aa, Human immunodeficiency virus 1
DNSNNTS

SEQ ID NO: 13 - protein, 9 aa, Human immunodeficiency virus 1
GNNNNDSSS

SEQ ID NO: 14 - protein, 7 aa, Human immunodeficiency virus 1
DNNNSSE

SEQ ID NO: 15 - protein, 7 aa, Human immunodeficiency virus 1
NENNSSQ

SEQ ID NO: 16 - protein, 7 aa, Human immunodeficiency virus 1
NNNNSSE

SEQ ID NO: 17 - protein, 17 aa, Human immunodeficiency virus 1
WNNNSTQSSNSTNSNDT

SEQ ID NO: 18 - protein, 16 aa, Human immunodeficiency virus 1
WNNNSTTNNNTNTNDT

SEQ ID NO: 19 - protein, 15 aa, Human immunodeficiency virus 1
YNGNGNNNNTNSNST

SEQ ID NO: 20 - protein, 16 aa, Human immunodeficiency virus 1
WNNNTTTNNTTNNNET

SEQ ID NO: 21 - protein, 13 aa, Human immunodeficiency virus 1
LLNNNNNTTNNDT

SEQ ID NO: 22 - protein, 14 aa, Human immunodeficiency virus 1
CIGNETTTEGCNGT

SEQ ID NO: 23 - protein, 16 aa, Human immunodeficiency virus 1
WNNNTTNNNNTESNDT

SEQ ID NO: 24 - protein, 16 aa, Human immunodeficiency virus 1
WNNNSTNNNNTNSNDT

SEQ ID NO: 25 - protein, 6 aa, Human immunodeficiency virus 1
NNNNTN

SEQ ID NO: 26 - protein, 7 aa, Human immunodeficiency virus 1
NNNTNTT

SEQ ID NO: 27 - protein, 7 aa, Human immunodeficiency virus 1
NNNNNTT

SEQ ID NO: 28 - protein, 6 aa, Human immunodeficiency virus 1
NNNSSN

SEQ ID NO: 29 - protein, 7 aa, Human immunodeficiency virus 1
NNNNTNN

SEQ ID NO: 30 - protein, 6 aa, Human immunodeficiency virus 1
NNNNTN

SEQ ID NO: 31 - protein, 6 aa, Human immunodeficiency virus 1
NNNSTN

SEQ ID NO: 32 - protein, 6 aa, Human immunodeficiency virus 1
NNNNTN

SEQ ID NO: 33 - protein, 12 aa, synthetic construct
SNVNGSGGNATE

SEQ ID NO: 34 - protein, 15 aa, synthetic construct
SNVNGSGNITGDMKE

SEQ ID NO: 35 - protein, 13 aa, synthetic construct
TDLNGSGGSMEKG

SEQ ID NO: 36 - protein, 12 aa, synthetic construct
TDLNGSGTMEKG

SEQ ID NO: 37 - protein, 8 aa, synthetic construct
TNVNGSGE

SEQ ID NO: 38 - protein, 13 aa, synthetic construct
TNVNGSGNDTMKE

SEQ ID NO: 39 - protein, 11 aa, synthetic construct
TDWKGNGSGDI

SEQ ID NO: 40 - protein, 11 aa, synthetic construct
TDWKGNGSEDI

SEQ ID NO: 41 - protein, 13 aa, synthetic construct
TNVNGSGNGSATE

SEQ ID NO: 42 - protein, 13 aa, synthetic construct
TNVTGNGTVDEKE

SEQ ID NO: 43 - protein, 20 aa, synthetic construct
TNANLTGSGSGNGSIGNITD

SEQ ID NO: 44 - protein, 19 aa, synthetic construct
TNANLTGNGSGGSIGNITD

SEQ ID NO: 45 - protein, 12 aa, synthetic construct
SNVNGSGGNMTG

SEQ ID NO: 46 - protein, 15 aa, synthetic construct
SDVNGSGNISSEMKG

SEQ ID NO: 47 - protein, 13 aa, synthetic construct
TNVNGSGNGSVTE

SEQ ID NO: 48 - protein, 13 aa, synthetic construct
TNVNGSGNGSEKE

SEQ ID NO: 49 - protein, 5 aa, synthetic construct
DNGSE

SEQ ID NO: 50 - protein, 5 aa, synthetic construct
NNGSE

SEQ ID NO: 51 - protein, 4 aa, synthetic construct
DNGS

SEQ ID NO: 52 - protein, 4 aa, synthetic construct
DNTS

SEQ ID NO: 53 - protein, 5 aa, synthetic construct
DNGSE

SEQ ID NO: 54 - protein, 5 aa, synthetic construct
NNGSE

SEQ ID NO: 55 - protein, 5 aa, synthetic construct
DDNGS

SEQ ID NO: 56 - protein, 4 aa, synthetic construct
DNTS

SEQ ID NO: 57 - protein, 5 aa, synthetic construct
DDNGS

SEQ ID NO: 58 - protein, 4 aa, synthetic construct
DNSS

SEQ ID NO: 59 - protein, 5 aa, synthetic construct
DNGSE

SEQ ID NO: 60 - protein, 5 aa, synthetic construct
GNSSE

SEQ ID NO: 61 - protein, 5 aa, synthetic construct
DNGSE

SEQ ID NO: 62 - protein, 5 aa, synthetic construct
DNSSQ

SEQ ID NO: 63 - protein, 5 aa, synthetic construct
NNGSE

SEQ ID NO: 64 - protein, 13 aa, synthetic construct
WGNGSGNGSGNDT

SEQ ID NO: 65 - protein, 15 aa, synthetic construct
WNGNGSGNGTESNDT

SEQ ID NO: 66 - protein, 13 aa, synthetic construct
WGNGSGNGSGNDT

SEQ ID NO: 67 - protein, 15 aa, synthetic construct
WNGNGSGNGSGSNDT

SEQ ID NO: 68 - protein, 13 aa, synthetic construct
YGNGSGNGSGNST

SEQ ID NO: 69 - protein, 15 aa, synthetic construct
YNGNGSGNGSGSNST

SEQ ID NO: 70 - protein, 13 aa, synthetic construct
WGNGSGNGSGNET

SEQ ID NO: 71 - protein, 15 aa, synthetic construct
WNGNGSGNGSGSNDT

SEQ ID NO: 72 - protein, 11 aa, synthetic construct
SLSGNGSGNDT

SEQ ID NO: 73 - protein, 12 aa, synthetic construct
LLNGSGNGSNDT

SEQ ID NO: 74 - protein, 13 aa, synthetic construct
WGNGSGNGSGNGT

SEQ ID NO: 75 - protein, 15 aa, synthetic construct
WNGNGSGNGSGSNGT

SEQ ID NO: 76 - protein, 13 aa, synthetic construct
WNGSGNGSGSNDT

SEQ ID NO: 77 - protein, 15 aa, synthetic construct
WNGNGSGNGSGSNDT

SEQ ID NO: 78 - protein, 13 aa, synthetic construct
WGNGSGNGSGNDT

SEQ ID NO: 79 - protein, 15 aa, synthetic construct
WNGNGSGNGSGSNDT

SEQ ID NO: 80 - protein, 5 aa, synthetic construct
GNGSN

SEQ ID NO: 81 - protein, 5 aa, synthetic construct
NNGTN

SEQ ID NO: 82 - protein, 5 aa, synthetic construct
GNGSN

SEQ ID NO: 83 - protein, 5 aa, synthetic construct
NNGTT

SEQ ID NO: 84 - protein, 5 aa, synthetic construct
GNGST

SEQ ID NO: 85 - protein, 5 aa, synthetic construct
NNGTT

SEQ ID NO: 86 - protein, 5 aa, synthetic construct
ANGSN

SEQ ID NO: 87 - protein, 5 aa, synthetic construct
ANSSN

SEQ ID NO: 88 - protein, 5 aa, synthetic construct
NNGSN

SEQ ID NO: 89 - protein, 5 aa, synthetic construct
NNGTN

SEQ ID NO: 90 - protein, 5 aa, synthetic construct
GNGSN

SEQ ID NO: 91 - protein, 5 aa, synthetic construct
NNGTN

SEQ ID NO: 92 - protein, 5 aa, synthetic construct
NNGSN

SEQ ID NO: 93 - protein, 5 aa, synthetic construct
NNGTN

SEQ ID NO: 94 - protein, 5 aa, synthetic construct
GNGSN

SEQ ID NO: 95 - protein, 5 aa, synthetic construct
NNGSN

SEQ ID NO: 96 - protein, 843 aa, synthetic construct
MRVMGTQRNCQNLLRWGTMILGMIIICSAAENLWVTVYYGVPVWKDAETTLFCASDAKAYETEKHNVWATHACVPTDPNPQEIPLENVTEEFNMWKNNMVEQMHTDIISLWDQSLKPCVKLTPLCVTLNCSNVNGSGGNATEEIKNCSFNMTTELRDKKQKVYSLFYRLDIVQIDNGSEYRLINCNTSAITQACPKVSFEPIPIHYCAPAGFAILKCNDKEFNGTGPCKNVSTVQCTHGIKPVVSTQLLLNGSLAEGEVMIRSENITNNAKNIIVQLNEPVKINCTRPNNNTRKSVHIGPGQAFYATGDIIGDIRQAHCNVSRTAWNKTLQQVAKQLRKYFGNKTIIFNSSSGGDLEITTHSFNCGGEFFYCNTSGLFNSTWGNGSGNGSGNDTITLPCRIKQIINMWQRVGQAMYAPPIPGVIRCVSNITGLILTRDGGGNGSNETFRPGGGDMRDNWRSELYKYKVVKIEPLGVAPTRARRRVVEREKRAVGLGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQDEIWDNMTWLQWDKEISNYTQIIYNLIEESQNQQEKNEQDLLALDKWASLWNWFDISNWLWYIKIFIMIVGGLIGLRIVFAVLSIINRVRQGYSPLSFQTHTPNPGGLDRPGRIEEEGGEQGRDRSIRLVSGFLALAWDDLRSLCLFSYHRLRDFILIAARTVELLGHSSLKGLRLGWEGLKYLWNLLLYWGRELKISAINLLDTIAIAVAGWTDRVIEIGQRIGRAILHIPRRIRQGLERALL

SEQ ID NO: 97 - protein, 836 aa, synthetic construct
MRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNGSGGSMEKGEIKNCSFNITTSIRDKVQKEYALFYKLDVVPIDNGSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIIFNQSSGGDPEIVMHSFNCGGEFFYCNTTQLFNSTWGNGSGNGSGNDTITLPCRIKQIINMWQEVGKAMYAPPIRGQIRCSSNITGLLLTRDGGGNGSNETFRPGGGDMRDNWRSELYKYKVVKIEPLGVAPTKAKRRVVQREKRAVGLGAMFLGFLGAAGSTMGAASITLTVQARQLLSGIVQQQNNLLRAIEAQQHLLQLTVWGIKQLQARVLAVERYLKDQQLLGIWGCSGKLICTTAVPWNTSWSNKSLDEIWDNMTWMQWEREIDNYTGLIYTLIEESQNQQEKNEQELLELDKWASLWNWFDITNWLWYIKIFIMIVGGLIGLRIVFAVLSIVNRVRQGYSPLSFQTRLPAPRGPDRPEGIEEEGGERDRDRSGRLVDGFLALIWVDLRSLCLFSYHRLRDLLLIVARIVELLGRRGWEALKYWWNLLQYWSQELKNSAVSLLNATAIAVAEGTDRIIEVVQRAGRAILHIPRRIRQGLERALL

SEQ ID NO: 98 - protein, 839 aa, synthetic construct
MRVRGILRNYQQWWIWGILGFWMLMICNVVGNLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNGSGEEMKNCSFNATTELRDKKKKVYALFYKLDIVPLDNGSEYRLINCNTSTITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISESKWNKTLQRVSKKLAEHFPNKTIKFNPSSGGDLEITTHSFNCRGEFFYCNTSKLFNSTYGNGSGNGSGNSTITLPCRIKQIINMWQEVGRAMYAPPIAGNITCKSNITGLLLTRDGGGNGSTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKSQDDIWDNMTWMQWDREISNYTNTIYRLLEESQNQQEKNEKDLLALDSWNNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILVTARAVELLGRSSLRGLQRGWEALKYLGSLVQYWGLELKKSAISLLDTIAIAVAEGTDRIIELIQRICRAIRNIPRRIRQGFEAALQ

SEQ ID NO: 99 - protein, 835 aa, synthetic construct
MRVRGIKRNYQHLWRWGTMLLGMLMTCSVAEQLWVTVYYGVPVWKEATTTLFCASDAKSYKAEAHNIWATHACVPTDPNPQEIELENVTENFNMWKNNMVEQMHEDIISLWDQSLKPCVKLTPLCVTLNCTDWKGNGSGDIEMKNCSFNITTEVRDKKKQVYALFYKLDVVQIDDNGSYRLINCNTSAITQACPKVSFEPIPIHYCAPAGFAILKCNDKKFNGTGPCKNVSTVQCTHGIKPVVSTQLLLNGSLAEEEIIIRSENLTNNAKIIIVQLNESVTINCTRPYNNTRQSIHIGPGQAFYTTGDIIGDIRQAHCNISGTDWNKTLQQVAKKLGDLLNKTTIIFKPSSGGDPEITTHSFNCGGEFFYCNTSGLFNSTWGNGSGNGSGNETITLPCRIKQIINMWQGVGKAMYAPPIEGLIKCSSNITGLLLTRDGGANGSNETFRPGGGDMRDNWRSELYKYKVVRIEPLGLAPTKAKRRVVEREKRAIGLGAMFLGFLGAAGSTMGAASLTLTVQARQLLSGIVQQQNNLLRAIEAQQHLLQLTVWGIKQLQARVLAVERYLKDQQLLGIWGCSGKHICTTNVPWNSSWSNKSLDEIWNNMTWMEWEREIDNYTGLIYSLIEESQIQQEKNEQELLELDKWASLWNWFSITNWLWYIKIFIMIVGGLIGLRIVFAVLSLVNRVRQGYSPLSFQTLLPAPRGPDRPEGIEEEGGEQGRGRSIRLVNGFSALIWDDLRNLCLFSYHRLRDLLLIAARIVELLGRRGWEALKYLWNLLQYWIQELKNSAISLLNTTAIAVAEGTDRVIEIVQRAGRAILNIPRRIRQGLERALL

SEQ ID NO: 100 - protein, 842 aa, synthetic construct
MRVKGIQRNWQHLWTWWTLILGLVIICSASNNLWVTVYYGVPVWEDADTPLFCASDAKAYSTEKHNVWATHACVPTDPNPQEIPLENVTENFNMWKNNMVEQMHEDIISLWDESLKPCVKLTPLCVTLNCTNVNGSGNGSATEELKNCSFNITTEIRDKQKKEYALFYKLDVVPIDDNGSYRLINCNVSTIKQACPKVSFEPIPIHYCAPAGFAILKCNDKNFNGTGPCKNVSTVQCTHGIKPVVSTQLLLNGSLAEEKIVIRSENITDNTKVIIVQLNETIEINCTRPNNNTRKSIRIGPGQAFYATGDIIGDIRQAHCNVSRTKWNEMLQKVKEQLKKIFNNKNITFNSSSGGDLEITTHSFNCRGEFFYCNTSGLFNNSSLSGNGSGNDTITLPCRIKQIVRMWQRVGQAMYAPPIAGNITCRSNITGLLLTRDGGNNGSNETFRPGGGDMRDNWRSELYKYKIVKIKPLGIAPTKARRRVVQREKRAVGLGAVFLGFLGAAGSTMGAASITLTVQVRQLLSGIVQQQSNLLRAIEAQQHLLQLTVWGIKQLQARVLAVERYLKDQQLLGIWGCSGKLICTTNVPWNTSWSNKSYNEIWDNMTWIQWEREINNYTQQIYSLIEESQNQQEKNEQDLLALDKWASLWNWFDISNWLWYIKIFIMIVGGLIGLRIVFAVLSIVNRVRQGYSPLSFQTLTHHQREPDRPEGIEEGGGEQDRDRSIRLVSGFLALAWDDLRSLCLFSYHRLRDFILIAARTVELLGRSSLQGLRLGWEGLKYLWNLLLYWGQELKNSAINLLDTIAIAVANWTDRVIEVAQRAGRAILNIPRRIRQGLERALL

SEQ ID NO: 101 - protein, 851 aa, synthetic construct
MRVKETQMNWPNLWKWGTLILGLVIICSASDNLWVTVYYGVPVWRDADTTLFCASDAKAHETEVHNVWATHACVPTDPNPQEIHLENVTENFNMWKNNMVEQMQEDVISLWDQSLKPCVKLTPLCVTLNCTNANLTGSGSGNGSIGNITDEVKNCSFNMTTEIRDKKQKVYALFYKLDIVQIDNGSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVEINCTRPSNNTRTSIRIGPGQVFYRTGDIIGDIRKAYCEINGTKWNETLKQVAEKLKEHFNNKTIIFQPPSGGDLEITMHHFNCRGEFFYCNTTKLFNSTWGNGSGNGSGNGTIILPCRIKQIINMWQGVGQAMYAPPISGIINCVSNITGILLTRDGGGNGSNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHMLQLTVWGIKQLQARVLAVERYLKDQKFLGLWGCSGKIICTTAVPWNSTWSNKSYEEIWNNMTWIEWEREISNYTNQIYEILTESQNQQDKNEKDLLELDKWASLWNWFDITNWLWYIKIFIMIVGGLIGLRIIFAVLSIVNRVRQGYSPLSFQTPTHHQREPDRPERIEEGGGEQGRDRSVRLVSGFLALAWDDLRSLCLFSYHRLRDFILIAARTVELLGHSSLKGLRRGWEGLKYLGNLLLYWGQELKTSAISLLDATAIAVAGWTDRVIEVAQRAWRAILHIPRRIRQGLERALL

SEQ ID NO: 102 - protein, 842 aa, synthetic construct
MRVMGTQRNYPLLWRWGMIIFWIIIICNAENLWVTVYYGVPVWRDAETTLFCASDAKAYDTEVHNVWATHACVPTDPNPQEIHLENVTENFNMWKNNMVEQMHEDIISLWDQSLKPCVKLTPLCVTLNCSNVNGSGGNMTGEIKNCSFNMTTELRDKKQKVYALFYRLDVVQIDNGSEYRLINCNTSAITQACPKVSFEPIPIHYCAPAGFAILKCNDKEFNGTGPCKNVSTVQCTHGIKPVVSTQLLLNGSLAEEEIVIRSENITNNAKTIIVQLNKPVKINCTRPNNNTRKSVRIGPGQTFYATGDIIGDIRQAHCNVSRTEWNNTLQKVATQLRKHFNNKTIIFANSSGGDLEITTHSFNCGGEFFYCNTSGLFNSTWNGSGNGSGSNDTITLQCRIKQIINMWQRVGQAMYAPPIPGVIRCESNITGLLLTRDGGNNGSNETFRPGGGDMRDNWRSELYKYKVVKIEPLGVAPTHAKRRVVEREKRAVGLGAVFLGFLGAAGSTMGAASITLTVQARQLLSGIVQQQSNLLKAIEAQQHLLRLTVWGIKQLQARVLALERYLRDQQLLGIWGCSGKLICTTNVPWNSSWSNKTYNDIWDNMTWLQWDKEISNYTDIIYNLIEESQNQQEKNEQDLLALDKWASLWNWFDITNWLWYIKIFIMIVGGLIGLRIVFAVLAIINRVRQGYSPLSFQTLTHHQREPDRPERIEEGGGEQDRDRSVRLVSGFLALAWDDLRSLCLFSYHRLRDFVLIAARTVELLGHSSLKGLRLGWEALKYLWNLLSYWGQELKNSAINLLDTIAIAVANWTDRIIEIGQRAGRAILNIPRRIRQGLERALL

SEQ ID NO: 103 - protein, 844 aa, synthetic construct
MRVRGIQRNYQHLWRWGTLILGMLIICSAAENLWVTVYYGVPVWKDAETTLFCASDAKAYETEVHNVWATHACVPTDPNPQEIVLENVTENFNMWKNNMVEQMHEDIISLWDQSLKPCVKLTPLCVTLNCTNVNGSGNGSVTEEIKNCSFNMTTELRDKKQKVYALFYKLDIVQINNGSEYRLINCNTSTITQACPKVSFEPIPIHYCAPAGFAILKCNDKNFNGTGPCKNVSTVQCTHGIKPVVSTQLLLNGSLAEEEIVIRSENITNNAKTIIVQLNESVEINCTRPNNNTRKSIRIGPGQAFYATGDIIGDIRQAHCNISRTKWNKTLQQVAKKLRKHFNNKTIIFNSSSGGDLEITTHSFNCGGEFFYCNTSGLFNSTWGNGSGNGSGNDTITLPCRIKQIINMWQRVGQAMYAPPIAGNIRCESNITGLLLTRDGGGNGSNETFRPGGGDMRDNWRSELYKYKVVKIEPLGVAPTKAKRRVVEREKRAVGLGAVFLGFLGAAGSTMGAASITLTVQARQLLSGIVQQQSNLLRAIEAQQHLLQLTVWGIKQLQARVLAVERYLKDQQLLGIWGCSGKLICTTNVPWNSSWSNKSLDEIWDNMTWMQWEREISNYTNIIYRLIEESQNQQEKNEQDLLALDKWASLWNWFDITNWLWYIKIFIMIVGGLIGLRIVFAVLSIVNRVRQGYSPLSFQTLTPHPRGPDRPERIEEEGGEQGRDRSIRLVSGFLALAWDDLRSLCLFSYHRLRDFILIAARTVELLGRSSLKGLRRGWEGLKYLWNLLQYWGQELKNSAISLLDTIAIAVAEWTDRVIEIGQRAGRAILNIPRRIRQGLERALL

SEQ ID NO: 104 - protein, 860 aa, Human immunodeficiency virus 1
MRVKETQMNWPNLWKWGTLILGLVIICSASDNLWVTVYYGVPVWRDADTTLFCASDAKAHETEVHNVWATHACVPTDPNPQEIHLENVTENFNMWKNNMVEQMQEDVISLWDQSLKPCVKLTPLCVTLNCTNANLTNTNTTNTNNTSNIGNITDEVRNCTFNMTTEIRDKKQKVYALFYKLDIVPIDNNNNSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVEINCTRPSNNTRTSIRIGPGQVFYRTGDIIGDIRKAYCEINGTKWNETLKQVAEKLKEHFNNKTIIFQPPSGGDLEITMHHFNCRGEFFYCNTTKLFNSTWNGNGNTTEGCNGTIILPCRIKQIINMWQGVGQAMYAPPISGIINCVSNITGILLTRDGGNNNNTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSYEEIWNNMTWIEWEREISNYTNQIYEILTESQNQQDKNEKDLLELDKWASLWNWFDITNWLWYIKIFIMIVGGLIGLRIIFAVLSIVNRVRQGYSPLSFQTPTHHQREPDRPERIEEGGGEQGRDRSVRLVSGFLALAWDDLRSLCLFSYHRLRDFILIAARTVELLGHSSLKGLRRGWEGLKYLGNLLLYWGQELKTSAISLLDATAIAVAGWTDRVIEVAQRAWRAILHIPRRIRQGLERALL

SEQ ID NO: 105 - protein, 852 aa, synthetic construct
MRVKETQMNWPNLWKWGTLILGLVIICSASDNLWVTVYYGVPVWRDADTTLFCASDAKAHETEVHNVWATHACVPTDPNPQEIHLENVTENFNMWKNNMVEQMQEDVISLWDQSLKPCVKLTPLCVTLNCTNANLTGNGSGGSIGNITDEVRNCTFNMTTEIRDKKQKVYALFYKLDIVPIGNSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVEINCTRPSNNTRTSIRIGPGQVFYRTGDIIGDIRKAYCEINGTKWNETLKQVAEKLKEHFNNKTIIFQPPSGGDLEITMHHFNCRGEFFYCNTTKLFNSTWNGNGSGNGSGSNGTIILPCRIKQIINMWQGVGQAMYAPPISGIINCVSNITGILLTRDGGNNGTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSYEEIWNNMTWIEWEREISNYTNQIYEILTESQNQQDKNEKDLLELDKWASLWNWFDITNWLWYIKIFIMIVGGLIGLRIIFAVLSIVNRVRQGYSPLSFQTPTHHQREPDRPERIEEGGGEQGRDRSVRLVSGFLALAWDDLRSLCLFSYHRLRDFILIAARTVELLGHSSLKGLRRGWEGLKYLGNLLLYWGQELKTSAISLLDATAIAVAGWTDRVIEVAQRAWRAILHIPRRIRQGLERALL

SEQ ID NO: 106 - protein, 836 aa, synthetic construct
MRVRGIKRLYQHLWRWGTMLLGMLMICSVAENLWVTVYYGVPVWKEATTTLFCASDAKSYKAEAHNIWATHACVPTDPNPQEIELENVTENFNMWKNNMVEQMHEDIISLWDQSLKPCVKLTPLCVTLNCTDWKGNGSEDIEMKNCSFNITTEIRDKKKQVYALFYKLDVVQIDNTSYRLINCNTSAITQACPKVTFEPIPIHYCAPAGFAILKCNDKKFNGTGPCKNVSTVQCTHGIKPVVSTQLLLNGSLAEEEIIIRSENLTNNAKIIIVQLNESVTINCTRPYNNTRQSIHIGPGQAFYTTNDIIGDIRQAHCNISGTEWNKTLQQVAKKLGDLLNKTTIIFKPSSGGDPEITTHSFNCGGEFFYCNTSGLFNSTWNGNGSGNGSGSNDTITLPCRIKQIINMWQGVGKAMYAPPIEGLIKCSSNITGLLLTRDGGANSSNETFRPGGGDMRDNWRSELYKYKVVRIEPLGLAPTKAKRRVVEREKRAIGLGAMFLGFLGAAGSTMGAASLTLTVQARQLLSGIVQQQNNLLRAIEAQQHLLQLTVWGIKQLQARVLAVERYLKDQQLLGIWGCSGKHICTTNVPWNSSWSNKSLDEIWNNMTWMEWEREIDNYTGLIYSLIEESQIQQEKNEQELLELDKWASLWNWFSITNWLWYIKIFIMIVGGLIGLRIVFAVLSLVNRVRQGYSPLSFQTLLPAPRGPDRPEGIEEEGGEQGRGRSIRLVNGFSALIWDDLRNLCLFSYHRLRDLLLIAARIVELLGRRGWEALKYLWNLLQYWIQELKNSAISLLNTTAIAVAEGTDRVIEIVQRAGRAILNIPRRIRQGLERALL

SEQ ID NO: 107 - protein, 852 aa, Human immunodeficiency virus 1
MRVRGIKRLYQHLWRWGTMLLGMLMICSVAENLWVTVYYGVPVWKEATTTLFCASDAKSYKAEAHNIWATHACVPTDPNPQEIELENVTENFNMWKNNMVEQMHEDIISLWDQSLKPCVKLTPLCVTLNCTDWKNNNTTNTTTNNTTSEDIEMKNCSFNITTEIRDKKKQVYALFYKLDVVQIDNSNNTSYRLINCNTSAITQACPKVTFEPIPIHYCAPAGFAILKCNDKKFNGTGPCKNVSTVQCTHGIKPVVSTQLLLNGSLAEEEIIIRSENLTNNAKIIIVQLNESVTINCTRPYNNTRQSIHIGPGQAFYTTNDIIGDIRQAHCNISGTEWNKTLQQVAKKLGDLLNKTTIIFKPSSGGDPEITTHSFNCGGEFFYCNTSGLFNSTWNNNTTTNNTTNNNDTITLPCRIKQIINMWQGVGKAMYAPPIEGLIKCSSNITGLLLTRDGGNNNNSSNETFRPGGGDMRDNWRSELYKYKVVRIEPLGLAPTKAKRRVVEREKRAIGLGAMFLGFLGAAGSTMGAASLTLTVQARQLLSGIVQQQNNLLRAIEAQQHLLQLTVWGIKQLQARVLAVERYLKDQQLLGIWGCSGKHICTTNVPWNSSWSNKSLDEIWNNMTWMEWEREIDNYTGLIYSLIEESQIQQEKNEQELLELDKWASLWNWFSITNWLWYIKIFIMIVGGLIGLRIVFAVLSLVNRVRQGYSPLSFQTLLPAPRGPDRPEGIEEEGGEQGRGRSIRLVNGFSALIWDDLRNLCLFSYHRLRDLLLIAARIVELLGRRGWEALKYLWNLLQYWIQELKNSAISLLNTTAIAVAEGTDRVIEIVQRAGRAILNIPRRIRQGLERALL

SEQ ID NO: 108 - protein, 846 aa, synthetic construct
MRVRGILRNYQQWWIWGILGFWMLMICNVVGNLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNGSGNDTMKEEMKNCSFNATTEIRDKKKKVYALFYKLDIVPLNNGSEYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISESKWNKTLQRVSKKLKEHFPNKTIKFNPSSGGDLEITTHSFNCRGEFFYCNTSKLFNGTYNGNGSGNGSGSNSTITLPCRIKQIINMWQEVGRAMYAPPIAGNITCKSNITGLLLTRDGGNNGTTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKSQEEIWDNMTWMQWDREISNYTDTIYRLLEESQNQQEKNEKDLLALDSWNNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILVTARAVELLGRSSLRGLQRGWEALKYLGSLVQYWGLELKKSAISLLDTIAIAVAEGTDRIIELIQRICRAIRNIPRRIRQGFEAALQ

SEQ ID NO: 109 - protein, 861 aa, Human immunodeficiency virus 1
MRVRGILRNYQQWWIWGILGFWMLMICNVVGNLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNTTNNNTNNNMKEEMKNCSFNATTEIRDKKKKVYALFYKLDIVPLNNENNNSSEYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISESKWNKTLQRVSKKLKEHFPNKTIKFNPSSGGDLEITTHSFNCRGEFFYCNTSKLFNGTYNGNGNTTNSTSNSTITLPCRIKQIINMWQEVGRAMYAPPIAGNITCKSNITGLLLTRDGGNNNNNNTTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKSQEEIWDNMTWMQWDREISNYTDTIYRLLEESQNQQEKNEKDLLALDSWNNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILVTARAVELLGRSSLRGLQRGWEALKYLGSLVQYWGLELKKSAISLLDTIAIAVAEGTDRIIELIQRICRAIRNIPRRIRQGFEAALQ

SEQ ID NO: 110 - protein, 847 aa, synthetic construct
MRVMRIQRNYPLLWRWGMIIFWIMIICNAENLWVTVYYGVPVWRDAETTLFCASDAKAYDTEVHNVWATHACVPTDPNPQEIHLENVTENFNMWKNNMVEQMHEDIISLWDQSLKPCVKLTPLCVTLNCSDVNGSGNISSEMKGEIKNCSFNMTTELRDKKQKVYALFYRLDVVQIDNSSQYRLINCNTSAITQACPKVSFEPIPIHYCAPAGFAILKCNDKEFNGTGPCKNVSTVQCTHGIKPVVSTQLLLNGSLAEEEIVIRSENITNNAKTIIVQLNEPVKINCTRPNNNTRKSVRIGPGQTFYATGDIIGDIRQAHCNVSKTEWNNTLQKVATQLRKYFNNKTIIFANSSGGDLEITTHSFNCGGEFFYCNTSGLFNSTWNGNGSGNGSGSNDTITLQCRIKQIINMWQRVGQAMYAPPIPGVIRCESNITGLLLTRDGGNNGTNETFRPGGGDMRDNWRSELYKYKVVKIEPLGVAPTHAKRRVVEREKRAVGLGAVFLGFLGAAGSTMGAASITLTVQARQLLSGIVQQQSNLLKAIEAQQHLLKLTVWGIKQLQARVLALERYLRDQQLLGIWGCSGKLICTTTVPWNSSWSNKTYNDIWDNMTWLQWDKEISNYTDIIYNLIEESQNQQEKNEQDLLALDKWASLWNWFDITNWLWYIKIFIMIVGGLIGLRIVFAVLNIINRVRQGYSPLSFQTLTHHQREPDRPERIEEGGGEQDRDRSVRLVSGFLALAWDDLRSLCLFSYHRLRDFVLIAARTVELLGHSSLKGLRLGWEALKYLWNLLSYWGQELKNSAINLLDTIAIAVANWTDRVIEIGQRAGRAILNIPRRIRQGLERALL

SEQ ID NO: 111 - protein, 770 aa, Human immunodeficiency virus 1
MRVMRIQRNYPLLWRWGMIIFWIMIICNAENLWVTVYYGVPVWRDAETTLFCASDAKAYDTEVHNVWATHACVPTDPNPQEIHLENVTENFNMWKNNMVEQMHEDIISLWDQSLKPCVKLTPLCVTLNCSDVNNSSSNSNNSNISSEMKGEIKNCSFNMTTELRDKKQKVYALFYRLDVVQIDESSNSSQYRLINCNTSAITQACPKVSFEPIPIHYCAPAGFAILKCNDKEFNGTGPCKNVSTVQCTHGIKPVVSTQLLLNGSLAEEEIVIRSENITNNAKTIIVQLNEPVKINCTRPNNNTRKSVRIGPGQTFYATGDIIGDIRQAHCNVSKTEWNNTLQKVATQLRKYFNNKTIIFANSSGGDLEITTHSFNCGGEFFYCNTSGLFNNTWNNNTTNNNNNTNSNDTITLQCRIKQIINMWQRVGQAMYAPPIPGVIRCESNITGLLLTRDGGNNNSTNETFRPGGGDMRDNWRSELYKYKVVKIEPLGVAPTHAKRRVVEREKRAVGLGAVFLGFLGAAGSTMGAASITLTVQARQLLSGIVQQQSNLLKAIEAQQHLLKLTVWGIKQLQARVLALERYLRDQQLLGIWGCSGKLICTTTVPWNSSWSNKTYNDIWDNMTWLQWDKEISNYTDIIYNLIEESQNQQEKNEQDLLALDKWASLWNWFDITNWLWYIKIFIMIVGGLIGLRIVFAVLNIINRVRQGYSPLSFQTLTHHQREPDRPERIEEGGGEQDRDRSVRLVSGFLALAWDDLRSLCLFSYHRLRDF

SEQ ID NO: 112 - protein, 848 aa, synthetic construct
MRVMGTQRNCQNLLRWGTMILGMIIICSAAENLWVTVYYGVPVWKDAETTLFCASDAKAYETEKHNVWATHACVPTDPNPQEIPLENVTEEFNMWKNNMVEQMHTDIISLWDQSLKPCVKLTPLCVTLNCSNVNGSGNITGDMKEEMKNCSFNMTTELRDKKQKVYSLFYKLDIVQINNGSEYRLINCNTSAITQACPKVSFEPIPIHYCAPAGFAILKCNDKEFNGTGPCKNVSTVQCTHGIKPVVSTQLLLNGSLAEGEVMIRSENITDNAKNIIVQLNEPVKINCTRPNNNTRKSVRIGPGQAFYATGDIIGDIRQAHCNVSKSEWNKTLQQVAKQLRKYFGNKTIIFTNSSGGDLEITTHSFNCGGEFFYCNTSGLFNSTWNGNGSGNGTESNDTITLPCRIKQIINMWQRVGQAMYAPPIPGVIRCVSNITGLILTRDGGNNGTNETFRPGGGDMRDNWRSELYKYKVVKIEPLGVAPTRAKRRVVEREKRAVGLGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSYDEIWDNMTWLQWDKEISNYTQIIYRLIEESQNQQEKNEQDLLALDKWASLWNWFDISNWLWYIKIFIMIVGGLIGLRIVFAVLSIINRVRQGYSPLSFQTHTPNPGGLDRPGRIEEEGGEQDRDRSIRLVSGFLALAWDDLRSLCLFSYHRLRDFILIAARTVELLGHSSLKGLRLGWEGLKYLWNLLLYWGRELKISAINLFDTIAIAVAGWTDRVIEIGQRIGRAILHIPRRIRQGLERALL

SEQ ID NO: 113 - protein, 861 aa, Human immunodeficiency virus 1
MRVMGTQRNCQNLLRWGTMILGMIIICSAAENLWVTVYYGVPVWKDAETTLFCASDAKAYETEKHNVWATHACVPTDPNPQEIPLENVTEEFNMWKNNMVEQMHTDIISLWDQSLKPCVKLTPLCVTLNCSNVNTNNTNTNSTITGDMKEEMKNCSFNMTTELRDKKQKVYSLFYKLDIVQINENSNNNSSEYRLINCNTSAITQACPKVSFEPIPIHYCAPAGFAILKCNDKEFNGTGPCKNVSTVQCTHGIKPVVSTQLLLNGSLAEGEVMIRSENITDNAKNIIVQLNEPVKINCTRPNNNTRKSVRIGPGQAFYATGDIIGDIRQAHCNVSKSEWNKTLQQVAKQLRKYFGNKTIIFTNSSGGDLEITTHSFNCGGEFFYCNTSGLFNSTWNNNGTNSSNSTESNDTITLPCRIKQIINMWQRVGQAMYAPPIPGVIRCVSNITGLILTRDGGNNNNTNETFRPGGGDMRDNWRSELYKYKVVKIEPLGVAPTRAKRRVVEREKRAVGLGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSYDEIWDNMTWLQWDKEISNYTQIIYRLIEESQNQQEKNEQDLLALDKWASLWNWFDISNWLWYIKIFIMIVGGLIGLRIVFAVLSIINRVRQGYSPLSFQTHTPNPGGLDRPGRIEEEGGEQDRDRSIRLVSGFLALAWDDLRSLCLFSYHRLRDFILIAARTVELLGHSSLKGLRLGWEGLKYLWNLLLYWGRELKISAINLFDTIAIAVAGWTDRVIEIGQRIGRAILHIPRRIRQGLERALL

SEQ ID NO: 114 - protein, 842 aa, synthetic construct
MRVKGIQRNWQHLWKWGTLILGLMIICSASENLWVTVYYGVPVWEDADTPLFCASDAKAYSTEKHNVWATHACVPTDPNPQEIPLENVTENFNMWKNNMVEQMHEDIISLWDESLKPCVKLTPLCVTLNCTNVTGNGTVDEKEELKNCSFNITTEIRDKKKKEYALFYKLDVVPIDNSSYRLINCNVSTIKQACPKVSFDPIPIHYCAPAGFAILKCRDKNFNGTGPCKNVSTVQCTHGIKPVVSTQLLLNGSLAEEEIVIRSENITDNAKIIIVQLNETIEINCTRPNNNTRKSIRIGPGQAFYATGDIIGDIRQAHCNVSKTKWNEMLQKVKTKLKEYFNNKNITFASSSGGDLEITTHSFNCRGEFFYCNTSGLFNNSLLNGSGNGSNDTITLPCRIKQIIRMWQRVGQAMYAPPIAGNITCRSNITGLLLTRDGGNNGTNETFRPGGGDMRDNWRSELYKYKVVKIKPLGVAPTKARRRVVGREKRAVGLGAVFLGFLGAAGSTMGAASITLTVQVRQLLSGIVQQQSNLLRAIEAQQHLLKLTVWGIKQLQARVLAVERYLKDQQLLGIWGCSGKLICTTTVPWNTSWSNKSYEEIWDNMTWIQWEREISNYTQQIYSLIEESQIQQEKNEQDLLALDKWASLWNWFDISNWLWYIKIFIMIVGGLIGLRIVFAVLNIVNRVRQGYSPLSFQTLTHHQREPDRPERIEEGGGEQDRDRSIRLVSGFLALAWDDLRSLCLFSYHRLRDFVLIAARTVELLGRSSLQGLRLGWEGLKYLWNLLLYWGQELKNSAINLLDTVAIAVANWTDRVIEVAQRAGRAILNIPRRIRQGLERALL

SEQ ID NO: 115 - protein, 797 aa, Human immunodeficiency virus 1
MRVKGIQRNWQHLWKWGTLILGLMIICSASENLWVTVYYGVPVWEDADTPLFCASDAKAYSTEKHNVWATHACVPTDPNPQEIPLENVTENFNMWKNNMVEQMHEDIISLWDESLLFYKLDVVPINNNNSSSYRLINCNVSTIKQACPKVSFDPIPIHYCAPAGFAILKCRDKNFNGTGPCKNVSTVQCTHGIKPVVSTQLLLNGSLAEEEIVIRSENITDNAKIIIVQLNETIEINCTRPNNNTRKSIRIGPGQAFYATGDIIGDIRQAHCNVSKTKWNEMLQKVKTKLKEYFNNKNITFASSSGGDLEITTHSFNCRGEFFYCNTSGLFNNSLLNNNNNSTNDTITLPCRIKQIIRMWQRVGQAMYAPPIAGNITCRSNITGLLLTRDGGNNNNNTNETFRPGGGDMRDNWRSELYKYKVVKIKPLGVAPTKARRRVVGREKRAVGLGAVFLGFLGAAGSTMGAASITLTVQVRQLLSGIVQQQSNLLRAIEAQQHLLKLTVWGIKQLQARVLAVERYLKDQQLLGIWGCSGKLICTTTVPWNTSWSNKSYEEIWDNMTWIQWEREISNYTQQIYSLIEESQIQQEKNEQDLLALDKWASLWNWFDISNWLWYIKIFIMIVGGLIGLRIVFAVLNIVNRVRQGYSPLSFQTLTHHQREPDRPERIEEGGGEQDRDRSIRLVSGFLALAWDDLRSLCLFSYHRLRDFVLIAARTVELLGRSSLQGLRLGWEGLKYLWNLLLYWGQELKNSAINLLDTVAIAVANWTDRVIEVAQRAGRAILNIPRRIRQGLERALL

SEQ ID NO: 116 - protein, 837 aa, synthetic construct
MRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNGSGTMEKGEIKNCSFNITTSIRDKVQKEYALFYKLDVVPIDNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIVFNQSSGGDPEIVMHSFNCGGEFFYCNTTQLFNSTWNGNGSGNGSGSNDTITLPCRIKQIINMWQEVGKAMYAPPIRGQIRCSSNITGLLLTRDGGNNGTTETFRPGGGDMRDNWRSELYKYKVVKIEPLGVAPTKAKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTAVPWNTSWSNKSLDEIWDNMTWMQWEREIDNYTGLIYTLIEESQNQQEKNEQELLELDKWASLWNWFDITNWLWYIKIFIMIVGGLIGLRIVFTVLSIVNRVRQGYSPLSFQTRLPAPRGPDRPEGIEEEGGERDRDRSGRLVDGFLALIWVDLRSLCLFSYHRLRDLLLIVARIVELLGRRGWEALKYWWNLLQYWSQELKNSAVSLLNATAIAVAEGTDRIIEVVQRAGRAILHIPRRIRQGLERALL

SEQ ID NO: 117 - protein, 855 aa, Human immunodeficiency virus 1
MRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNNTTTNNTNSSSSSTMEKGEIKNCSFNITTSIRDKVQKEYALFYKLDVVPIDNNNNNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIVFNQSSGGDPEIVMHSFNCGGEFFYCNTTQLFNSTWNNNSTNTNNNTNTNDTITLPCRIKQIINMWQEVGKAMYAPPIRGQIRCSSNITGLLLTRDGGNNNTNTTETFRPGGGDMRDNWRSELYKYKVVKIEPLGVAPTKAKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTAVPWNTSWSNKSLDEIWDNMTWMQWEREIDNYTGLIYTLIEESQNQQEKNEQELLELDKWASLWNWFDITNWLWYIKIFIMIVGGLIGLRIVFTVLSIVNRVRQGYSPLSFQTRLPAPRGPDRPEGIEEEGGERDRDRSGRLVDGFLALIWVDLRSLCLFSYHRLRDLLLIVARIVELLGRRGWEALKYWWNLLQYWSQELKNSAVSLLNATAIAVAEGTDRIIEVVQRAGRAILHIPRRIRQGLERALL

SEQ ID NO: 118 - protein, 846 aa, synthetic construct
MRVRGIQRNYQHLWRWGTLILGMLIICSAAENLWVTVYYGVPVWKDAETTLFCASDAKAYETEVHNVWATHACVPTDPNPQEIVLENVTENFNMWKNNMVEQMHEDIISLWDQSLKPCVKLTPLCVTLNCTNVNGSGNGSEKEEIKNCSFNMTTELRDKKQKVYALFYKLDIVQINNGSEYRLINCNTSTITQACPKVSFEPIPIHYCAPAGFAILKCNDKNFNGTGPCKNVSTVQCTHGIKPVVSTQLLLNGSLAEEEIVIRSENITNNAKTIIVQLNESVEINCTRPNNNTRKSIRIGPGQAFYATGDIIGDIRQAHCNISKTKWNKTLQQVAKKLRKYFNNKTIIFNSSSGGDLEITTHSFNCGGEFFYCNTSGLFNSTWNGNGSGNGSGSNDTITLPCRIKQIINMWQRVGQAMYAPPIAGNIRCESNITGLLLTRDGGNNGSNETFRPGGGDMRDNWRSELYKYKVVKIEPLGVAPTKAKRRVVEREKRAVGLGAVFLGFLGAAGSTMGAASITLTVQARQLLSGIVQQQSNLLRAIEAQQHLLQLTVWGIKQLQARVLAVERYLKDQQLLGIWGCSGKLICTTNVPWNSSWSNKSLDEIWDNMTWMQWEREISNYTNIIYRLIEESQNQQEKNEQDLLELDKWASLWNWFDITNWLWYIKIFIMIVGGLIGLRIVFAVLSIVNRVRQGYSPLSFQTLTPHPRGPDRPERIEEEGGEQGRDRSIRLVSGFLALAWDDLRSLCLFSYHRLRDFILIAARTVELLGRSSLKGLRRGWEGLKYLWNLLQYWGQELKNSAISLLDTTAIAVAEWTDRVIEIVQRAGRAILNIPRRIRQGLERALL

SEQ ID NO: 119 - protein, 859 aa, Human immunodeficiency virus 1
MRVRGIQRNYQHLWRWGTLILGMLIICSAAENLWVTVYYGVPVWKDAETTLFCASDAKAYETEVHNVWATHACVPTDPNPQEIVLENVTENFNMWKNNMVEQMHEDIISLWDQSLKPCVKLTPLCVTLNCTNVNNTNTNNTNSNNTSEEKEEIKNCSFNMTTELRDKKQKVYALFYKLDIVQINNNNNSSSYRLINCNTSTITQACPKVSFEPIPIHYCAPAGFAILKCNDKNFNGTGPCKNVSTVQCTHGIKPVVSTQLLLNGSLAEEEIVIRSENITNNAKTIIVQLNESVEINCTRPNNNTRKSIRIGPGQAFYATGDIIGDIRQAHCNISKTKWNKTLQQVAKKLRKYFNNKTIIFNSSSGGDLEITTHSFNCGGEFFYCNTSGLFNSTWNNNTNNNNTNSNDTITLPCRIKQIINMWQRVGQAMYAPPIAGNIRCESNITGLLLTRDGGNNNNNTNETFRPGGGDMRDNWRSELYKYKVVKIEPLGVAPTKAKRRVVEREKRAVGLGAVFLGFLGAAGSTMGAASITLTVQARQLLSGIVQQQSNLLRAIEAQQHLLQLTVWGIKQLQARVLAVERYLKDQQLLGIWGCSGKLICTTNVPWNSSWSNKSLDEIWDNMTWMQWEREISNYTNIIYRLIEESQNQQEKNEQDLLELDKWASLWNWFDITNWLWYIKIFIMIVGGLIGLRIVFAVLSIVNRVRQGYSPLSFQTLTPHPRGPDRPERIEEEGGEQGRDRSIRLVSGFLALAWDDLRSLCLFSYHRLRDFILIAARTVELLGRSSLKGLRRGWEGLKYLWNLLQYWGQELKNSAISLLDTTAIAVAEWTDRVIEIVQRAGRAILNIPRRIRQGLERALL

SEQ ID NO: 120 - protein, 35 aa, synthetic construct
CTRPNNNTRKSIHIGPGRAFYATGDIIGDIRQAHC

SEQ ID NO: 121 - protein, 4 aa, synthetic construct
MEKG

SEQ ID NO: 122 - protein, 6 aa, synthetic construct
IGNITD

SEQ ID NO: 123 - protein, 19 aa, Human immunodeficiency virus 1
NVNTNNTNTNSTITGDMKE

SEQ ID NO: 124 - protein, 22 aa, Human immunodeficiency virus 1
TDLNNTTTNNTNSSSSSTMEKG

SEQ ID NO: 125 - protein, 21 aa, Human immunodeficiency virus 1
TNVNVTNTNTTNNNTNNNMKE

SEQ ID NO: 126 - protein, 21 aa, Human immunodeficiency virus 1
TDWKNNNTTNTTTNNTTSEDI

SEQ ID NO: 127 - protein, 22 aa, Human immunodeficiency virus 1
TNVTNNNTNNNNNNNTTVDEKE

SEQ ID NO: 128 - protein, 24 aa, Human immunodeficiency virus 1
TNANLTNTNTTNTNNTSNIGNITD

SEQ ID NO: 129 - protein, 21 aa, Human immunodeficiency virus 1
SDVNNSSSNSNNSNISSEMKG

SEQ ID NO: 130 - protein, 21 aa, Human immunodeficiency virus 1
TNVNNTNTNNTNSNNTSEEKE

SEQ ID NO: 131 - protein, 10 aa, Human immunodeficiency virus 1
NENSNNNSSE

SEQ ID NO: 132 - protein, 8 aa, Human immunodeficiency virus 1
DNNNNNTS

SEQ ID NO: 133 - protein, 9 aa, Human immunodeficiency virus 1
NNENNNSSE

SEQ ID NO: 134 - protein, 7 aa, Human immunodeficiency virus 1
DNSNNTS

SEQ ID NO: 135 - protein, 7 aa, Human immunodeficiency virus 1
NNNNSSS

SEQ ID NO: 136 - protein, 8 aa, Human immunodeficiency virus 1
DNNNNSSE

SEQ ID NO: 137 - protein, 8 aa, Human immunodeficiency virus 1
DESSNSSQ

SEQ ID NO: 138 - protein, 8 aa, Human immunodeficiency virus 1
NNNNNSSS

SEQ ID NO: 139 - protein, 17 aa, Human immunodeficiency virus 1
WNNNGTNSSNSTESNDT

SEQ ID NO: 140 - protein, 17 aa, Human immunodeficiency virus 1
WNNNSTNTNNNTNTNDT

SEQ ID NO: 141 - protein, 15 aa, Human immunodeficiency virus 1
YNGNGNTTNSTSNST

SEQ ID NO: 142 - protein, 16 aa, Human immunodeficiency virus 1
WNNNTTTNNTTNNNDT

SEQ ID NO: 143 - protein, 12 aa, Human immunodeficiency virus 1
LLNNNNNSTNDT

SEQ ID NO: 144 - protein, 14 aa, Human immunodeficiency virus 1
WNGNGNTTEGCNGT

SEQ ID NO: 145 - protein, 17 aa, Human immunodeficiency virus 1
WNNNTTNNNNNTNSNDT

SEQ ID NO: 146 - protein, 15 aa, Human immunodeficiency virus 1
WNNNTNNNNTNSNDT

SEQ ID NO: 147 - protein, 8 aa, Human immunodeficiency virus 1
NNNNNNTT

SEQ ID NO: 148 - protein, 7 aa, Human immunodeficiency virus 1
NNNNSSN

SEQ ID NO: 149 - protein, 7 aa, Human immunodeficiency virus 1
NNNNNTN

SEQ ID NO: 150 - protein, 7 aa, Human immunodeficiency virus 1
NNNNNTN